(19) United States
(12) Patent Application Publication
(10) Pub. No.: US 2024/0124865 A1
(43) Pub. Date: Apr. 18, 2024

(54) ADAMTS13 VARIANT

(71) Applicant: The University of Manchester, Manchester Greater Manchester (GB)

(72) Inventors: Stuart McRae Allan, Manchester Greater Manchester (GB); Kieron South, Manchester Greater Manchester (GB)

(21) Appl. No.: 18/277,434

(22) PCT Filed: Feb. 17, 2022
(86) PCT No.: PCT/GB2022/050427

§ 371 Date: Aug. 16, 2023

(30) Foreign Application Priority Data
Feb. 17, 2021 (GB) 2102208.2

(51) Int. Cl. C12N 9/64; A61P 7/02; C12N 15/63

(52) U.S. Cl. CPC C12N 9/6489; A61P 7/02; C12N 15/63; C12Y 304/24087

(57) ABSTRACT

The disclosure provides improved ADAMTS13 variants with amino acid substitutions in the linker 3 region. The disclosure also provides methods for producing and using the variants.

### TECHNICAL FIELD

The present disclosure relates to the fields of molecular biology and enzymology. The present disclosure also relates to methods of medical treatment and prophylaxis.

### BACKGROUND

ADAMTS13 (adisintegrin-like and metalloprotease with thrombospondin type 1 motif 13) is a protease which regulates blood homeostasis by cleaving von Willebrand factor (VWF). Specifically, ADAMTS13 is a zinc-containing metalloprotease enzyme that cleaves VWF for breakdown.

VWF is an adhesive plasma glycoprotein that circulates as large globular multimers (Sadler et al., 2009). In this large globular multimer conformation, VWF is unable to capture platelets. The conformation of VWF is changed when bound at sites of vessel damage; this conformational change is driven by the shear force of flowing blood to enable platelet capture.

The biological breakdown of VWF is largely mediated by ADAMTS13, ADAMTS13 cleaves VWF between tyrosine at position 842 and methionine at position 843 (or 1605-1606 of the gene) in the A2 domain.

This breaks down VWF into smaller units, which are degraded by other peptidases.

VWF is required for normal platelet adhesion, but exaggerated VWF adhesive activity has been proposed to cause thrombotic thrombocytopenic purpura (TTP) (Moake et al., 1982). Additionally, ADAMTS13 and VWF have been implicated as important factors in other thrombotic diseases such as stroke (De Meyer et al., 2012).

Thrombus formation is central to the occurrence of acute ischemic stroke. Current antithrombotics used to treat or prevent cerebral ischemia are only moderately effective or bear an increased risk of severe bleeding (De Meyer et al. 2012). There is only one licensed thrombolytic therapy for the treatment of acute ischaemic stroke, recombinant tissue plasminogen activator (rt-PA), which is not widely administered due to an associated risk of haemorrhagic transformation (Wang et al. 2014). Moreover, resistance to rt-PA thrombolysis is observed in 40-50% of patients which is believed to result from varying clot composition.

Administration of rt-PA has been the standard practice of care for 25 years (National Institute of Neurological and Stroke 1995). Retrospective analysis of CT angiograms show rt-PA treatment has minimal effect on large proximal occlusions located in the internal carotid artery or basilar artery (Bhatia et al. 2010). In addition, recent data from the DIRECT-MT clinical trial shows endovascular thrombectomy alone is non-inferior to combined rt-PA administration and endovascular thrombectomy (Yang et al. 2020). Moreover, a landscape of associated risks evolving over the years have caused drastic refinements in the guidelines of rt-PA use, specifying complex eligibility criteria and a narrow window in which administration is safe (0-4.5 h), causing restricted access for many (Whiteley et al. 2016; Emberson et al. 2014). Therefore uncertainty in the safety and effectiveness of rt-PA prompts demand for novel thrombolytic agents in AIS.

A proportion of stroke patients present with thrombi particularly rich in VWF (Denorme et al. 2016) and a high VWF:ADAMTS13 ratio is not only predisposing for AIS it is also consistent with poorer outcome and increased mortality (Taylor et al. 2020; Sonneveld et al. 2015). Interestingly, in a murine model of middle cerebral artery (MCA) ischaemic stroke, administration of recombinant ADAMTS13 was shown to be effective in the dissolution of VWF-rich occlusions which were resistant to rt-PA treatment (Denorme et al. 2016). This result is in line with previous studies which report ADAMTS13 deletion is detrimental to infarct size and neurological outcomes post-stroke whereas VWF−/− animals are protected (Fujioka et al. 2010; Kleinschnitz et al. 2009).

The VWF-ADAMTS13 axis is also known to play a central role in thrombo-inflammation, a process which is increasingly recognised as exacerbating infarct development, now a therapeutic target in itself (Stoll and Nieswandt 2019). In non-Inflammatory conditions, ADAMTS13 deficiency leads to increased VWF dependent endothelial activation, P-selectin upregulation and leukocyte rolling. In inflamed vessels, increased leukocyte extravation and adhesion is observed (Chauhan et al. 2008). Likewise, using the murine filament model of ischemic stroke, ADAMTS13 deficiency enhances immune cell infiltration, neutrophil extravasation and proinflammatory cytokine release in the ipsilateral brain hemisphere (Khan et al. 2012). Additionally, VWF has been shown to co-localise with neutrophil extracellular traps (NETs), known promotors of platelet recruitment and thrombogenesis (Martinod and Wagner 2014). In a murine model of MRSA induced liver injury, ADAMTS13 administration was demonstrated to free VWF-dependent NET adhesion to the inflamed vessel wall (Kolaczkowska et al. 2015).

In terms of safety, the use of rt-PA increases the risk of intracerebral haemorrhage In up to 7% of AIS patients (Yaghi et al. 2017). The risk of haemorrhage rises proportionally with stroke severity and delay in rt-PA administration (Whiteley et al. 2016). Before administration of rt-PA, risk factors which increase the likelihood of haemorrhagic transformation must first be ruled out during which time the efficacy of rt-PA diminishes. In a murine model of haemorrhagic stroke, ADAMTS13 administration has no effect on bleeding and may, in fact, be of therapeutic potential In this condition (Zhao et al. 2009).

Recombinant ADAMTS13 (rADAMTS13) has systemic antithrombotic activity in ADAMTS13^{-/-} mice (De Meyer et al., 2012b). Additionally, rADAMTS-13 has been shown to have protective effects in murine stroke models without the risk of intracerebral haemorrhage associated with rt-PA (Nakano et al., 2015).

Whilst initial results for ADAMTS13 therapies were promising, the need for very high doses has hindered progress. This high dose requirement can be explained by a recently discovered ADAMTS13 activation mechanism. As wild type ADAMTS13 requires substrate-Induced conformational activation to achieve full activity, the administration of high doses is required to achieve an effective concentration of active ADAMTS13. Studies have found that ADAMTS13 circulates in a quiescent conformation, maintained by an autoinhibitory interaction between its N-terminal spacer domain and its C-terminal CUB domains (South et al., 2014).

It has been found that three linker regions in the distal domains of ADAMTS13 are important for flexibility and enable the interaction between the proximal and the T8-CUB2 domains during the inactive state (Deforche et al., 2015). More recent research performed binding and functional studies on a panel of truncated ADAMTS13 variants to develop a model for the conformational activation of structurally quiescent ADAMTS13 by VWF (South et al., 2017). Results indicated that both the isolated CUB1 and CUB2 domains in ADAMTS13 bind to the spacer domain exosite of a truncated ADAMTS13 variant. However, only the CUB1 domain inhibited proteolytic activity of the truncated ADAMTS13 variant. The combination of findings from this study support an ADAMTS13 activation model in which VWF D4-CK engages the TSP8-CUB2 domains, inducing the conformational change that disrupts the CUB1-spacer domain interaction and thereby activates ADAMTS13. This study therefore suggests that the most important domains for ADAMTS13 conformational activation are the two CUB domains and the spacer region.

ADAMTS13 variants have previously been generated with amino acid substitutions in the spacer region in an effort to prevent the need for conformational activation and overcome the problems with wild type ADAMTS13 therapy. The gain of function (GoF) ADAMTS13 variant (R568K/F592Y/R660K/Y661F/Y665F), was shown to have disrupted autoinhibition and enhanced proteolytic activity (Jian et al., 2012). Furthermore, this GoF variant restored cerebral blood flow at a lower dose than wild type ADAMTS-13 and retained some ability to recanalize vessels when administration was delayed by 1 h in a murine stroke model (South et al., 2018). However, the reduction in dose requirement demonstrated by the GoF mutant may not be sufficient to fully overcome the problem associated with wildtype ADAMTS13. Additionally, the efficacy of the GoF mutant is reduced when administration is delayed. The development of variants with a greater reduction in dose requirement and an improved efficacy after delayed administration could lead to a more clinically significant therapy.

The present disclosure has been devised in light of the above considerations. The inventors have identified that the linker 3 region of the ADAMTS13 protein is key to the conformational activation mechanism and have produced a number of improved ADAMTS13 variants with amino acid substitutions in the linker 3 region for the first time.

### SUMMARY

In a first aspect, the present disclosure provides an ADAMTS13 variant having an amino acid sequence comprising one or more amino acid substitutions in the region corresponding to SEQ ID NO:48 relative to the amino acid sequence of wildtype human ADAMTS13.

In some embodiments, the ADAMTS13 variant comprises substitution at one or more of the following positions relative to the amino acid sequence of wildtype human ADAMTS13: A1144, A1145, A1146, P1147, P1154, P1171, P1173, P1175, P1180, and P1182.

In some embodiments, an ADAMTS13 variant comprises substitution of an alanine residue with a valine, isoleucine, lysine, leucine, or methionine residue relative to the amino acid sequence of wildtype human ADAMTS13. In some embodiments, an ADAMTS13 variant comprises substitution of an alanine residue with a valine, isoleucine, or lysine residue relative to the amino acid sequence of wildtype human ADAMTS13.

In some embodiments, an ADAMTS13 variant comprises substitution of a proline residue with a valine, isoleucine, lysine, leucine, or methionine residue relative to the amino acid sequence of wildtype human ADAMTS13. In some embodiments, an ADAMTS13 variant comprises substitution of a praline residue with a valine, isoleucine, or lysine residue relative to the amino acid sequence of wildtype human ADAMTS13.

In some embodiments, the ADAMTS13 variant comprises an amino acid sequence according to SEQ ID NO:50 or 156.

In some embodiments, the ADAMTS13 variant comprises substitution to valine, isoleucine, or lysine at one or more of the following positions relative to the amino acid sequence of wildtype human ADAMTS13: A1144, A1145, A1146, P1147, P1154, P1171, P1173, P1175, P1180, and P1182.

In some embodiments, the ADAMTS13 variant comprises:
(i) the amino acid sequence of SEQ ID NO:51, 52, 53, 54, 55, 56, 57, 58, 59, or 60; or
(ii) the amino acid sequence of SEQ ID NO:136, 137, 138, 139, 140, 141, 142, 143, 144, or 145; or
(iii) the amino acid sequence of SEQ ID NO:146, 147, 148, 149, 150, 151, 152, 153, 154, or 155.

In some embodiments, the ADAMTS13 variant comprises: (i) the amino acid sequence of SEQ ID NO:51; or (ii) the amino acid sequence of SEQ ID NO:52; or (iii) the amino acid sequence of SEQ ID NO:53; or (iv) the amino acid sequence of SEQ ID NO:54; or (v) the amino acid sequence of SEQ ID NO:55; or (vi) the amino acid sequence of SEQ ID NO:56; or (vii) the amino acid sequence of SEQ ID NO:57; or (viii) the amino acid sequence of SEQ ID NO:58; or (ix) the amino acid sequence of SEQ ID NO:59; or (x) the amino acid sequence of SEQ ID NO:60.

In some embodiments, the ADAMTS13 variant comprises: (i) the amino acid sequence of SEQ ID NO:136; or (ii) the amino acid sequence of SEQ ID NO:137; or (iii) the amino acid sequence of SEQ ID NO:138; or (iv) the amino acid sequence of SEQ ID NO:139; or (v) the amino acid sequence of SEQ ID NO:140; or (vi) the amino acid sequence of SEQ ID NO:141; or (vii) the amino acid sequence of SEQ ID NO:142; or (viii) the amino acid sequence of SEQ ID NO:143; or (ix) the amino acid sequence of SEQ ID NO:144; or (x) the amino acid sequence of SEQ ID NO:145.

In some embodiments, the ADAMTS13 variant comprises: (i) the amino acid sequence of SEQ ID NO:146; or (ii) the amino acid sequence of SEQ ID NO:147; or (iii) the amino acid sequence of SEQ ID NO:148; or (iv) the amino acid sequence of SEQ ID NO:149; or (v) the amino acid sequence of SEQ ID NO:150; or (vi) the amino acid sequence of SEQ ID NO:151; or (vii) the amino acid sequence of SEQ ID NO:152; or (viii) the amino acid sequence of SEQ ID NO:153; or (ix) the amino acid sequence of SEQ ID NO:154; or (x) the amino acid sequence of SEQ ID NO:155.

In some embodiments, the ADAMTS13 variant comprises substitution to valine, isoleucine, or lysine at one or both of positions P1180 and/or P1182 relative to the amino acid sequence of wildtype human ADAMTS13.

In some embodiments, the ADAMTS13 variant comprises the amino acid sequence of SEQ ID NO:59, 60, 144, 145, 154, or 155.

In some embodiments, the ADAMTS13 variant comprises or consists of: (i) an amino acid sequence having at least 60% sequence identity to the amino acid sequence of SEQ ID NO:61; or (ii) an amino acid sequence having at least 60% sequence identity to the amino acid sequence of SEQ ID NO:62; or (iii) an amino acid sequence having at least 60% sequence identity to the amino acid sequence of SEQ ID NO:63; or (iv) an amino acid sequence having at least 60% sequence identity to the amino acid sequence of SEQ ID NO:64; or (v) an amino acid sequence having at least 60% sequence identity to the amino acid sequence of SEQ ID NO 65; or (vi) an amino acid sequence having at least 60% sequence identity to the amino acid sequence of SEQ ID NO:66; or (vii) an amino acid sequence having at least 60% sequence identity to the amino acid sequence of SEQ ID NO:67; or (viii) an amino acid sequence having at least 60% sequence identity to the amino acid sequence of SEQ ID NO 68; or (ix) an amino acid sequence having at least 60% sequence identity to the amino acid sequence of SEQ ID NO:69; or (x) an amino acid sequence having at least 60% sequence identity to the amino acid sequence of SEQ ID NO:70.

In some embodiments, the ADAMTS13 variant displays increased proteolytic activity as compared to wildtype human ADAMTS13.

The present disclosure also provides a nucleic acid encoding an ADAMTS13 variant according to the present disclosure.

The present disclosure also provides an expression vector, comprising a nucleic acid according to the present disclosure.

The present disclosure also provides a cell comprising an ADAMTS13 variant, a nucleic acid, or an expression vector according to the present disclosure.

The present disclosure also provides a method for producing an ADAMTS13 variant, comprising culturing a cell comprising a nucleic acid or expression vector according to the present disclosure, under conditions suitable for expression of an ADAMTS13 variant by the cell.

The present disclosure also provides a pharmaceutical composition comprising an ADAMTS13 variant, a nucleic acid, an expression vector, or a cell according to the present disclosure, and a pharmaceutically acceptable carrier, diluent, excipient or adjuvant.

The present disclosure also provides an ADAMTS13 variant, a nucleic acid, an expression vector, a cell, or a composition according to the present disclosure, for use in a method of medical treatment or prophylaxis.

The present disclosure also provides an ADAMTS13 variant, a nucleic acid, an expression vector, a cell, or a composition according to the present disclosure, for use in a method of treatment or prevention of a disease or condition characterised by one or more of: an increased level and/or activity of VWF, or of a complex comprising VWF; a reduced level of ADAMTS13; a reduced level of ADAMTS13 proteolytic activity; thrombosis; and inflammation.

The present disclosure also provides the use of an ADAMTS13 variant, a nucleic acid, an expression vector, a cell, or a composition according to the present disclosure in the manufacture of a medicament for use in a method of treatment or prevention of a disease or condition characterised by one or more of: an increased level and/or activity of VWF, or of a complex comprising VWF; a reduced level of ADAMTS13; a reduced level of ADAMTS13 proteolytic activity; thrombosis; and inflammation.

The present disclosure also provides a method of treating or preventing a disease or condition characterised by one or more of: an increased level and/or activity of VWF, or of a complex comprising VWF; a reduced level of ADAMTS13; a reduced level of ADAMTS13 proteolytic activity; thrombosis; and inflammation, comprising administering to a subject a therapeutically or prophylactically effective amount of an ADAMTS13 variant, a nucleic acid, an expression vector, a cell, or a composition according to the present disclosure.

In some embodiments, the disease or condition is selected from: a disease/condition characterised by thrombosis, a disease/condition characterised by inflammation, thrombotic thrombocytopenic purpura (TTP), ischaemic stroke, haemorrhagic stroke, subarachnoid haemorrhage (SAH), intracerebral haemorrhage (ICH), chronic thromboembolic pulmonary hypotension (CTEPH), myocardial infarction (MI), ST-elevation myocardial infarction (STEMI), unstable angina (UA), ischemia, reperfusion, deep venous thrombosis, pulmonary embolism, intravascular coagulation (DIC), hemolytic-uremic syndrome (HUS), cerebral infarction, systemic lupus erythematosus (SLE), disease cause by infection with a SARSr-CoV (e.g. SARS-CoV-2; e.g. COVID-19), acute respiratory distress syndrome (ARDS), pneumonia, kidney damage, nephropathy, microvascular diseases, dementia, Crohn's disease, inflammatory bowel disease, ulcerative colitis, and bacterial diarrhoea.

The present disclosure also provides a method of cleaving VWF, comprising contacting VWF or a complex comprising VWF with an ADAMTS13 variant according to the present disclosure.

The present disclosure includes the combination of the aspects and preferred features described herein except where such combinations are clearly impermissible or expressly avoided.

Sequences

| SEQ | | |
|---|---|---|
| ID | Description | Sequence |
| 1 | Human ADAMTS13 | MHQRHPRARCPPLCVAGILACGFLLGCWGPSHFQQSCLQALEPQAVSSYL |
| | isoform 1 (UniProt: | SPGAPLKGRPPSPGFQRQRQRQRRAAGGILHLELLVAVGPDVFQAHQEDT |
| | Q76LX8-1, v1) | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGT |
| 2 | Human ADAMTS13 | MHQRHPRARCPPLCVAGILACGFLLGCWGPSHFQQSCLQALEPQAVSSYL |
| | isoform 2 (UniProt: | SPGAPLKGRPPSPGFQRQRQRQRRAAGGILHLELLVAVGPDVFQAHQEDT |
| | Q76LX8-2) | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGACGRQHLEPTGTIDMR |
| | | GPGQADCAVAIGRPLGEVVTLRVLESSLNCSAGDMLLLWGRLTWRKMCRK |
| | | LLDMTFSSKTNTLVVRQRCGRPGGGVLLRYGSQLAPETFYRECDMQLFGP |
| | | WGEIVSPSLSPATSNAGGCRLFINVAPHARIAIHALATNMGAGTEGANAS |
| | | YILIRDTHSLRTTAFHGQQVLYWESESSQAEMEFSEGFLKAQASLRGQYW |
| | | TLQSWVPEMQDPQSWKGKEGT |
| 3 | Human ADAMTS13 | MHQRHPRARCPPLCVAGILACGFLLGCWGPSHFQQSCLQALEPQAVSSYL |
| | isoform 3 (UniProt: | SPGAPLKGRPPSPGFQRQRQRQRRAAGGILHLELLVAVGPDVFQAHQEDT |
| | Q76LX8-2) | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSANEQCRVAFGPKAVACTFAREHLDM |
| | | CQALSCHTDPLDQSSCSRLLVPLLDGTECGVEKWCSKGRCRSLVELTPIA |
| | | AVHGRWSSWGPRSPCSRSCGGGVVTRRRQCNNPRPAFGGRACVGADLQAE |
| | | MCNTQACEKTQLEFMSQQCARTDGQPLRSSPGGASFYHWGAAVPHSQGDA |
| | | LCRHMCRAIGESFIMKRGDSFLDGTRCMPSGPREDGTLSLCVSGSCRTFG |
| | | CDGRMDSQQVWDRCQVCGGDNSTCSPRKGSFTAGRAREYVTFLTVTPNLT |
| | | SVYIANHRPLFTHLAVRIGGRYVVAGKMSISPNTTYPSLLEDGRVEYRVA |
| | | LTEDRLPRLEEIRIWGPLQEDADIQVYRRYGEEYGNLTRPDITFTYFQPK |
| | | PRQAWVWAAVRGPCSVSCGAGLRWVNYSCLDQARKELVETVQCQGSQQPP |
| | | AWPEACVLEPCPPYWAVGDFGPCSASCGGGLRERPVRCVEAQGSLLKTLP |
| | | PARCRAGAQQPAVALETCNPQPCPARWEVSEPSSCTSAGGAGLALENETC |
| | | VPGADGLEAPVTEGPGSVDEKLPAPEPCVGMSCPPGWGHLDATSAGEKAP |
| | | SPWGSIRTGAQAAHVWTPAAGSCSVSCGRGLMELRFLCMDSALRVPVQEE |
| | | LCGLASKPGSRREVCQAVPCPARWQYKLAACSVSCGRGVVRRILYCARAH |
| | | GEDDGEEILLDTQCQGLPRPEPQEACSLEPCPPRWKVMSLGPCSASCGLG |
| | | TARRSVACVQLDQGQDVEVDEAACAALVRPEASVPCLIADCTYRWHVGTW |
| | | MECSVSCGDGIQRRRDTCLGPQAQAPVPADFCQHLPKPVTVRGCWAGPCV |
| | | GQGACGRQHLEPTGTIDMRGPGQADCAVAIGRPLGEVVTLRVLESSLNCS |
| | | AGDMLLLWGRLTWRKMCRKLLDMTFSSKTNTLVVRQRCGRPGGGVLLRYG |
| | | SQLAPETFYRECDMQLFGPWGEIVSPSLSPATSNAGGCRLFINVAPHARI |
| | | AIHALATNMGAGTEGANASYILIRDTHSLRTTAFHGQQVLYWESESSQAE |
| | | MEFSEGFLKAQASLRGQYWTLQSWVPEMQDPQSWKGKEGT |
| 4 | Human ADAMTS13 | MDMCQALSCHTDPLDQSSCSRLLVPLLDGTECGVEKWCSKGRCRSLVELT |
| | isoform 4 (UniProt: | PIAAVHGRWSSWGPRSPCSRSCGGGVVTRRRQCNNPRPAFGGRACVGADL |
| | Q76LX8-2) | QAEMCNTQACEKTQLEFMSQQCARTDGQPLRSSPGGASFYHWGAAVPHSQ |
| | | GDALCRHMCRAIGESFIMKRGDSFLDGTRCMPSGPREDGTLSLCVSGSCR |
| | | TFGCDGRMDSQQVWDRCQVCGGDNSTCSPRKGSFTAGRAREYVTFLTVTP |
| | | NLTSVYIANHRPLFTHLAVRIGGRYVVAGKMSISPNTTYPSLLEDGRVEY |
| | | RVALTEDRLPRLEEIRIWGPLQEDADIQVGGVRAQLMHISWWSRPGLGER |
| | | DLCARGRWPGGSSD |
| 5 | Human ADAMTS13 | MHQRHPRARCPPLCVAGILACGFLLGCWG |
| | isoform 1, 2, 3 signal | |
| | peptide (positions 1 | |
| | to 29 of UniProt: | |
| | Q76LX8-1, v1) | |
| 6 | Human ADAMTS13 | PSHFQQSCLQALEPQAVSSYLSPGAPLKGRPPSPGFQRQRQRQRR |
| | isoform 1, 2, 3 | |
| | propeptide (positions | |
| | 30 to 74 of UniProt: | |
| | Q76LX8-1, v1) | |
| 7 | Human ADAMTS13 | PSHFQQSCLQALEPQAVSSYLSPGAPLKGRPPSPGFQRQRQRQRRAAGGI |
| | isoform 1 lacking | LHLELLVAVGPDVFQAHQEDTERYVLTNLNIGAELLRDPSLGAQFRVHLV |
| | signal peptide | KMVILTEPEGAPNITANLTSSLLSVCGWSQTINPEDDTDPGHADLVLYIT |
| | (positions 30 to 1427 | RFDLELPDGNRQVRGVTQLGGACSPTWSCLITEDTGFDLGVTIAHEIGHS |
| | of UniProt: Q76LX8- | FGLEHDGAPGSGCGPSGHVMASDGAAPRAGLAWSPCSRRQLLSLLSAGRA |
| | 1, v1) | RCVWDPPRPQPGSAGHPPDAQPGLYYSANEQCRVAFGPKAVACTFAREHL |
| | | DMCQALSCHTDPLDQSSCSRLLVPLLDGTECGVEKWCSKGRCRSLVELTP |
| | | IAAVHGRWSSWGPRSPCSRSCGGGVVTRRRQCNNPRPAFGGRACVGADLQ |
| | | AEMCNTQACEKTQLEFMSQQCARTDGQPLRSSPGGASFYHWGAAVPHSQG |
| | | DALCRHMCRAIGESFIMKRGDSFLDGTRCMPSGPREDGTLSLCVSGSCRT |
| | | FGCDGRMDSQQVWDRCQVCGGDNSTCSPRKGSFTAGRAREYVTFLTVTPN |
| | | LTSVYIANHRPLFTHLAVRIGGRYVVAGKMSISPNTTYPSLLEDGRVEYR |
| | | VALTEDRLPRLEEIRIWGPLQEDADIQVYRRYGEEYGNLTRPDITFTYFQ |
| | | PKPRQAWVWAAVRGPCSVSCGAGLRWVNYSCLDQARKELVETVQCQGSQQ |
| | | PPAWPEACVLEPCPPYWAVGDFGPCSASCGGGLRERPVRCVEAQGSLLKT |
| | | LPPARCRAGAQQPAVALETCNPQPCPARWEVSEPSSCTSAGGAGLALENE |
| | | TCVPGADGLEAPVTEGPGSVDEKLPAPEPCVGMSCPPGWGHLDATSAGEK |
| | | APSPWGSIRTGAQAAHVWTPAAGSCSVSCGRGLMELRFLCMDSALRVPVQ |
| | | EELCGLASKPGSRREVCQAVPCPARWQYKLAACSVSCGRGVVRRILYCAR |
| | | AHGEDDGEEILLDTQCQGLPRPEPQEACSLEPCPPRWKVMSLGPCSASCG |
| | | LGTARRSVACVQLDQGQDVEVDEAACAALVRPEASVPCLIADCTYRWHVG |
| | | TWMECSVSCGDGIQRRRDTCLGPQAQAPVPADFCQHLPKPVTVRGCWAGP |
| | | CVGQGTPSLVPHEEAAAPGRTTATPAGASLEWSQARGLLFSPAPQPRRLL |
| | | PGPQENSVQSSACGRQHLEPTGTIDMRGPGQADCAVAIGRPLGEVVTLRV |
| | | LESSLNCSAGDMLLLWGRLTWRKMCRKLLDMTFSSKTNTLVVRQRCGRPG |
| | | GGVLLRYGSQLAPETFYRECDMQLFGPWGEIVSPSLSPATSNAGGCRLFI |
| | | NVAPHARIAIHALATNMGAGTEGANASYILIRDTHSLRTTAFHGQQVLYW |
| | | ESESSQAEMEFSEGFLKAQASLRGQYWTLQSWVPEMQDPQSWKGKEGT |
| 8 | Mature human | AAGGILHLELLVAVGPDVFQAHQEDTERYVLTNLNIGAELLRDPSLGAQF |
| | ADAMTS13 isoform | RVHLVKMVILTEPEGAPNITANLTSSLLSVCGWSQTINPEDDTDPGHADL |
| | 1 lacking signal | VLYITRFDLELPDGNRQVRGVTQLGGACSPTWSCLITEDTGFDLGVTIAH |
| | peptide and | EIGHSFGLEHDGAPGSGCGPSGHVMASDGAAPRAGLAWSPCSRRQLLSLL |
| | propeptide (positions | SAGRARCVWDPPRPQPGSAGHPPDAQPGLYYSANEQCRVAFGPKAVACTF |
| | 75 to 1427 of | AREHLDMCQALSCHTDPLDQSSCSRLLVPLLDGTECGVEKWCSKGRCRSL |
| | UniProt: Q76LX8-1, | VELTPIAAVHGRWSSWGPRSPCSRSCGGGVVTRRRQCNNPRPAFGGRACV |
| | v1) | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAAPGRTTATPAGASLEWSQARGLLFSPAPQ |
| | | PRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 9 | Human ADAMTS13 | PSHFQQSCLQALEPQAVSSYLSPGAPLKGRPPSPGFQRQRQRQRRAAGGI |
| | isoform 2 lacking | LHLELLVAVGPDVFQAHQEDTERYVLTNLNIGAELLRDPSLGAQFRVHLV |
| | signal peptide | KMVILTEPEGAPNITANLTSSLLSVCGWSQTINPEDDTDPGHADLVLYIT |
| | | RFDLELPDGNRQVRGVTQLGGACSPTWSCLITEDTGFDLGVTIAHEIGHS |
| | | FGLEHDGAPGSGCGPSGHVMASDGAAPRAGLAWSPCSRRQLLSLLSAGRA |
| | | RCVWDPPRPQPGSAGHPPDAQPGLYYSANEQCRVAFGPKAVACTFAREHL |
| | | DMCQALSCHTDPLDQSSCSRLLVPLLDGTECGVEKWCSKGRCRSLVELTP |
| | | IAAVHGRWSSWGPRSPCSRSCGGGVVTRRRQCNNPRPAFGGRACVGADLQ |
| | | AEMCNTQACEKTQLEFMSQQCARTDGQPLRSSPGGASFYHWGAAVPHSQG |
| | | DALCRHMCRAIGESFIMKRGDSFLDGTRCMPSGPREDGTLSLCVSGSCRT |
| | | FGCDGRMDSQQVWDRCQVCGGDNSTCSPRKGSFTAGRAREYVTFLTVTPN |
| | | LTSVYIANHRPLFTHLAVRIGGRYVVAGKMSISPNTTYPSLLEDGRVEYR |
| | | VALTEDRLPRLEEIRIWGPLQEDADIQVYRRYGEEYGNLTRPDITFTYFQ |
| | | PKPRQAWVWAAVRGPCSVSCGAGLRWVNYSCLDQARKELVETVQCQGSQQ |
| | | PPAWPEACVLEPCPPYWAVGDFGPCSASCGGGLRERPVRCVEAQGSLLKT |
| | | LPPARCRAGAQQPAVALETCNPQPCPARWEVSEPSSCTSAGGAGLALENE |
| | | TCVPGADGLEAPVTEGPGSVDEKLPAPEPCVGMSCPPGWGHLDATSAGEK |
| | | APSPWGSIRTGAQAAHVWTPAAGSCSVSCGRGLMELRFLCMDSALRVPVQ |
| | | EELCGLASKPGSRREVCQAVPCPARWQYKLAACSVSCGRGVVRRILYCAR |
| | | AHGEDDGEEILLDTQCQGLPRPEPQEACSLEPCPPRWKVMSLGPCSASCG |
| | | LGTARRSVACVQLDQGQDVEVDEAACAALVRPEASVPCLIADCTYRWHVG |
| | | TWMECSVSCGDGIQRRRDTCLGPQAQAPVPADFCQHLPKPVTVRGCWAGP |
| | | CVGQGACGRQHLEPTGTIDMRGPGQADCAVAIGRPLGEVVTLRVLESSLN |
| | | CSAGDMLLLWGRLTWRKMCRKLLDMTFSSKTNTLVVRQRCGRPGGGVLLR |
| | | YGSQLAPETFYRECDMQLFGPWGEIVSPSLSPATSNAGGCRLFINVAPHA |
| | | RIAIHALATNMGAGTEGANASYILIRDTHSLRTTAFHGQQVLYWESESSQ |
| | | AEMEFSEGFLKAQASLRGQYWTLQSWVPEMQDPQSWKGKEGT |
| 10 | Mature human | AAGGILHLELLVAVGPDVFQAHQEDTERYVLTNLNIGAELLRDPSLGAQF |
| | ADAMTS13 isoform | RVHLVKMVILTEPEGAPNITANLTSSLLSVCGWSQTINPEDDTDPGHADL |
| | 2 lacking signal | VLYITRFDLELPDGNRQVRGVTQLGGACSPTWSCLITEDTGFDLGVTIAH |
| | peptide and | EIGHSFGLEHDGAPGSGCGPSGHVMASDGAAPRAGLAWSPCSRRQLLSLL |
| | propeptide | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGACGRQHLEPTGTIDMRGPGQADCAVAIGRPLGEVVTLRVL |
| | | ESSLNCSAGDMLLLWGRLTWRKMCRKLLDMTFSSKTNTLVVRQRCGRPGG |
| | | GVLLRYGSQLAPETFYRECDMQLFGPWGEIVSPSLSPATSNAGGCRLFIN |
| | | VAPHARIAIHALATNMGAGTEGANASYILIRDTHSLRTTAFHGQQVLYWE |
| | | SESSQAEMEFSEGFLKAQASLRGQYWTLQSWVPEMQDPQSWKGKEGT |
| 11 | Human ADAMTS13 | PSHFQQSCLQALEPQAVSSYLSPGAPLKGRPPSPGFQRQRQRQRRAAGGI |
| | isoform 3 lacking | LHLELLVAVGPDVFQAHQEDTERYVLTNLNIGAELLRDPSLGAQFRVHLV |
| | signal peptide | KMVILTEPEGAPNITANLTSSLLSVCGWSQTINPEDDTDPGHADLVLYIT |
| | | RFDLELPDGNRQVRGVTQLGGACSPTWSCLITEDTGFDLGVTIAHEIGHS |
| | | FGLEHDGAPGSGCGPSGHVMASDGAAPRAGLAWSPCSRRQLLSLLSANEQ |
| | | CRVAFGPKAVACTFAREHLDMCQALSCHTDPLDQSSCSRLLVPLLDGTEC |
| | | GVEKWCSKGRCRSLVELTPIAAVHGRWSSWGPRSPCSRSCGGGVVTRRRQ |
| | | CNNPRPAFGGRACVGADLQAEMCNTQACEKTQLEFMSQQCARTDGQPLRS |
| | | SPGGASFYHWGAAVPHSQGDALCRHMCRAIGESFIMKRGDSFLDGTRCMP |
| | | SGPREDGTLSLCVSGSCRTFGCDGRMDSQQVWDRCQVCGGDNSTCSPRKG |
| | | SFTAGRAREYVTFLTVTPNLTSVYIANHRPLFTHLAVRIGGRYVVAGKMS |
| | | ISPNTTYPSLLEDGRVEYRVALTEDRLPRLEEIRIWGPLQEDADIQVYRR |
| | | YGEEYGNLTRPDITFTYFQPKPRQAWVWAAVRGPCSVSCGAGLRWVNYSC |
| | | LDQARKELVETVQCQGSQQPPAWPEACVLEPCPPYWAVGDFGPCSASCGG |
| | | GLRERPVRCVEAQGSLLKTLPPARCRAGAQQPAVALETCNPQPCPARWEV |
| | | SEPSSCTSAGGAGLALENETCVPGADGLEAPVTEGPGSVDEKLPAPEPCV |
| | | GMSCPPGWGHLDATSAGEKAPSPWGSIRTGAQAAHVWTPAAGSCSVSCGR |
| | | GLMELRFLCMDSALRVPVQEELCGLASKPGSRREVCQAVPCPARWQYKLA |
| | | ACSVSCGRGVVRRILYCARAHGEDDGEEILLDTQCQGLPRPEPQEACSLE |
| | | PCPPRWKVMSLGPCSASCGLGTARRSVACVQLDQGQDVEVDEAACAALVR |
| | | PEASVPCLIADCTYRWHVGTWMECSVSCGDGIQRRRDTCLGPQAQAPVPA |
| | | DFCQHLPKPVTVRGCWAGPCVGQGACGRQHLEPTGTIDMRGPGQADCAVA |
| | | IGRPLGEVVTLRVLESSLNCSAGDMLLLWGRLTWRKMCRKLLDMTFSSKT |
| | | NTLVVRQRCGRPGGGVLLRYGSQLAPETFYRECDMQLFGPWGEIVSPSLS |
| | | PATSNAGGCRLFINVAPHARIAIHALATNMGAGTEGANASYILIRDTHSL |
| | | RTTAFHGQQVLYWESESSQAEMEFSEGFLKAQASLRGQYWTLQSWVPEMQ |
| | | DPQSWKGKEGT |
| 12 | Mature human | AAGGILHLELLVAVGPDVFQAHQEDTERYVLTNLNIGAELLRDPSLGAQF |
| | ADAMTS13 isoform | RVHLVKMVILTEPEGAPNITANLTSSLLSVCGWSQTINPEDDTDPGHADL |
| | 3 lacking signal | VLYITRFDLELPDGNRQVRGVTQLGGACSPTWSCLITEDTGFDLGVTIAH |
| | peptide and | EIGHSFGLEHDGAPGSGCGPSGHVMASDGAAPRAGLAWSPCSRRQLLSLL |
| | propeptide | SANEQCRVAFGPKAVACTFAREHLDMCQALSCHTDPLDQSSCSRLLVPLL |
| | | DGTECGVEKWCSKGRCRSLVELTPIAAVHGRWSSWGPRSPCSRSCGGGVV |
| | | TRRRQCNNPRPAFGGRACVGADLQAEMCNTQACEKTQLEFMSQQCARTDG |
| | | QPLRSSPGGASFYHWGAAVPHSQGDALCRHMCRAIGESFIMKRGDSFLDG |
| | | TRCMPSGPREDGTLSLCVSGSCRTFGCDGRMDSQQVWDRCQVCGGDNSTC |
| | | SPRKGSFTAGRAREYVTFLTVTPNLTSVYIANHRPLFTHLAVRIGGRYVV |
| | | AGKMSISPNTTYPSLLEDGRVEYRVALTEDRLPRLEEIRIWGPLQEDADI |
| | | QVYRRYGEEYGNLTRPDITFTYFQPKPRQAWVWAAVRGPCSVSCGAGLRW |
| | | VNYSCLDQARKELVETVQCQGSQQPPAWPEACVLEPCPPYWAVGDFGPCS |
| | | ASCGGGLRERPVRCVEAQGSLLKTLPPARCRAGAQQPAVALETCNPQPCP |
| | | ARWEVSEPSSCTSAGGAGLALENETCVPGADGLEAPVTEGPGSVDEKLPA |
| | | PEPCVGMSCPPGWGHLDATSAGEKAPSPWGSIRTGAQAAHVWTPAAGSCS |
| | | VSCGRGLMELRFLCMDSALRVPVQEELCGLASKPGSRREVCQAVPCPARW |
| | | QYKLAACSVSCGRGVVRRILYCARAHGEDDGEEILLDTQCQGLPRPEPQE |
| | | ACSLEPCPPRWKVMSLGPCSASCGLGTARRSVACVQLDQGQDVEVDEAAC |
| | | AALVRPEASVPCLIADCTYRWHVGTWMECSVSCGDGIQRRRDTCLGPQAQ |
| | | APVPADFCQHLPKPVTVRGCWAGPCVGQGACGRQHLEPTGTIDMRGPGQA |
| | | DCAVAIGRPLGEVVTLRVLESSLNCSAGDMLLLWGRLTWRKMCRKLLDMT |
| | | FSSKTNTLVVRQRCGRPGGGVLLRYGSQLAPETFYRECDMQLFGPWGEIV |
| | | SPSLSPATSNAGGCRLFINVAPHARIAIHALATNMGAGTEGANASYILIR |
| | | DTHSLRTTAFHGQQVLYWESESSQAEMEFSEGFLKAQASLRGQYWTLQSW |
| | | VPEMQDPQSWKGKEGT |
| 13 | Human ADAMTS13 | LHLELLVAVGPDVFQAHQEDTERYVLTNLNIGAELLRDPSLGAQFRVHLV |
| | isoform 1, 2 | KMVILTEPEGAPNITANLTSSLLSVCGWSQTINPEDDTDPGHADLVLYIT |
| | metalloprotease | RFDLELPDGNRQVRGVTQLGGACSPTWSCLITEDTGFDLGVTIAHEIGHS |
| | domain | FGLEHDGAPGSGCGPSGHVMASDGAAPRAGLAWSPCSRRQLLSLLSAGRA |
| | | RCVWDPP |
| 14 | Human ADAMTS13 | RPQPGSAGHPPDAQPGLYYSANEQCRVAFGPKAVACTFAREHLDMCQALS |
| | isoform 1, 2 | CHTDPLDQSSCSRLLVPLLDGTECGVEKWCSKGRCRSLVELTPIAAV |
| | disintegrin-like | |
| | domain | |
| 15 | Human ADAMTS13 | HGRWSSWGPRSPCSRSCGGGVVTRRRQCNNPRPAFGGRACVGADLQAEMC |
| | isoform 1, 2, 3, 4 | NTQACE |
| | TSP-1 repeat 1 | |
| 16 | Human ADAMTS13 | KTQLEFMSQQCARTDGQPLRSSPGGASFYHWGAAVPHSQGDALCRHMCRA |
| | isoform 1, 2, 3, 4 | IGESFIMKRGDSFLDGTRCMPSGPREDGTLSLCVSGSCRTFGCDGRMDSQ |
| | cysteine-rich domain | QVWDRCQVCGGDNSTCS |
| 17 | Human ADAMTS13 | SPRKGSFTAGRAREYVTFLTVTPNLTSVYIANHRPLFTHLAVRIGGRYVV |
| | isoform 1, 2, 3 | AGKMSISPNTTYPSLLEDGRVEYRVALTEDRLPRLEEIRIWGPLQEDADI |
| | spacer domain | QVYRRYGEEYGNLTRPDITFTYFQPKPRQA |
| 18 | Human ADAMTS13 | PRQAWVWAAVRGPCSVSCGAGLRWVNYSCLDQARKELVETVQCQGSQQP |
| | isoform 1, 2, 3, 4 | |
| | TSP-1 repeat 2 | |
| 19 | Human ADAMTS13 | CPPYWAVGDFGPCSASCGGGLRERPVRCVEAQGSLLKTLPPARCRAGAQQ |
| | isoform 1, 2, 3 TSP-1 | PAVALETCNPQPCP |
| | repeat 3 | |
| 20 | Human ADAMTS13 | WEVSEPSSCTSAGGAGLALENETCVPGADGLEAPVTEGPGSVDEKLPAPE |
| | isoform 1, 2, 3 TSP-1 | PC |
| | repeat 4 | |
| 21 | Human ADAMTS13 | VWTPAAGSCSVSCGRGLMELRFLCMDSALRVPVQEELCGLASKPGSRREV |
| | isoform 1, 2, 3 TSP-1 | CQAVP |
| | repeat 5 | |
| 22 | Human ADAMTS13 | CPARWQYKLAACSVSCGRGVVRRILYCARAHGEDDGEEILLDTQCQGLPR |
| | isoform 1, 2, 3 TSP-1 | PEPQEACSLEP |
| | repeat 6 | |
| 23 | Human ADAMTS13 | CPPRWKVMSLGPCSASCGLGTARRSVACVQLDQGQDVEVDEAACAALVRP |
| | isoform 1, 2, 3 TSP-1 | EASVPCL |
| | repeat 7 | |
| 24 | Human ADAMTS13 | CTYRWHVGTWMECSVSCGDGIQRRRDTCLGPQAQAPVPADFCQHLPKPVT |
| | isoform 1, 2, 3 TSP-1 | VRGCWAGPCV |
| | repeat 8 | |
| 25 | Human ADAMTS13 | CGRQHLEPTGTIDMRGPGQADCAVAIGRPLGEVVTLRVLESSLNCSAGDM |
| | isoform 1, 2, 3 CUB | LLLWGRLTWRKMCRKLLDMTFSSKTNTLVVRQRCGRPGGGVLLRYGSQLA |
| | domain 1 | PETFYRE |
| 26 | Human ADAMTS13 | CDMQLFGPWGEIVSPSLSPATSNAGGCRLFINVAPHARIAIHALATNMGA |
| | isoform 1, 2, 3 CUB | GTEGANASYILIRDTHSLRTTAFHGQQVLYWESESSQAEMEFSEGFLKAQ |
| | domain 2 | ASLRGQYWTLQSWVPEMQDPQSWKGKEGT |
| 27 | Human VWF isoform | MIPARFAGVLLALALILPGTLCAEGTRGRSSTARCSLFGSDFVNTFDGSM |
| | 1 (UniProt: P04275- | YSFAGYCSYLLAGGCQKRSFSIIGDFQNGKRVSLSVYLGEFFDIHLFVNG |
| | 1, v4) | TVTQGDQRVSMPYASKGLYLETEAGYYKLSGEAYGFVARIDGSGNFQVLL |
| | | SDRYFNKTCGLCGNFNIFAEDDFMTQEGTLTSDPYDFANSWALSSGEQWC |
| | | ERASPPSSSCNISSGEMQKGLWEQCQLLKSTSVFARCHPLVDPEPFVALC |
| | | EKTLCECAGGLECACPALLEYARTCAQEGMVLYGWTDHSACSPVCPAGME |
| | | YRQCVSPCARTCQSLHINEMCQERCVDGCSCPEGQLLDEGLCVESTECPC |
| | | VHSGKRYPPGTSLSRDCNTCICRNSQWICSNEECPGECLVTGQSHFKSFD |
| | | NRYFTFSGICQYLLARDCQDHSFSIVIETVQCADDRDAVCTRSVTVRLPG |
| | | LHNSLVKLKHGAGVAMDGQDVQLPLLKGDLRIQHTVTASVRLSYGEDLQM |
| | | DWDGRGRLLVKLSPVYAGKTCGLCGNYNGNQGDDFLTPSGLAEPRVEDFG |
| | | NAWKLHGDCQDLQKQHSDPCALNPRMTRFSEEACAVLTSPTFEACHRAVS |
| | | PLPYLRNCRYDVCSCSDGRECLCGALASYAAACAGRGVRVAWREPGRCEL |
| | | NCPKGQVYLQCGTPCNLTCRSLSYPDEECNEACLEGCFCPPGLYMDERGD |
| | | CVPKAQCPCYYDGEIFQPEDIFSDHHTMCYCEDGFMHCTMSGVPGSLLPD |
| | | AVLSSPLSHRSKRSLSCRPPMVKLVCPADNLRAEGLECTKTCQNYDLECM |
| | | SMGCVSGCLCPPGMVRHENRCVALERCPCFHQGKEYAPGETVKIGCNTCV |
| | | CQDRKWNCTDHVCDATCSTIGMAHYLTFDGLKYLFPGECQYVLVQDYCGS |
| | | NPGTFRILVGNKGCSHPSVKCKKRVTILVEGGEIELFDGEVNVKRPMKDE |
| | | THFEVVESGRYIILLLGKALSVVWDRHLSISVVLKQTYQEKVCGLCGNFD |
| | | GIQNNDLTSSNLQVEEDPVDFGNSWKVSSQCADTRKVPLDSSPATCHNNI |
| | | MKQTMVDSSCRILTSDVFQDCNKLVDPEPYLDVCIYDTCSCESIGDCACF |
| | | CDTIAAYAHVCAQHGKVVTWRTATLCPQSCEERNLRENGYECEWRYNSCA |
| | | PACQVTCQHPEPLACPVQCVEGCHAHCPPGKILDELLQTCVDPEDCPVCE |
| | | VAGRRFASGKKVTLNPSDPEHCQICHCDVVNLTCEACQEPGGLVVPPTDA |
| | | PVSPTTLYVEDISEPPLHDFYCSRLLDLVFLLDGSSRLSEAEFEVLKAFV |
| | | VDMMERLRISQKWVRVAVVEYHDGSHAYIGLKDRKRPSELRRIASQVKYA |
| | | GSQVASTSEVLKYTLFQIFSKIDRPEASRITLLLMASQEPQRMSRNFVRY |
| | | VQGLKKKKVIVIPVGIGPHANLKQIRLIEKQAPENKAFVLSSVDELEQQR |
| | | DEIVSYLCDLAPEAPPPTLPPDMAQVTVGPGLLGVSTLGPKRNSMVLDVA |
| | | FVLEGSDKIGEADFNRSKEFMEEVIQRMDVGQDSIHVTVLQYSYMVTVEY |
| | | PFSEAQSKGDILQRVREIRYQGGNRTNTGLALRYLSDHSFLVSQGDREQA |
| | | PNLVYMVTGNPASDEIKRLPGDIQVVPIGVGPNANVQELERIGWPNAPIL |
| | | IQDFETLPREAPDLVLQRCCSGEGLQIPTLSPAPDCSQPLDVILLLDGSS |
| | | SFPASYFDEMKSFAKAFISKANIGPRLTQVSVLQYGSITTIDVPWNVVPE |
| | | KAHLLSLVDVMQREGGPSQIGDALGFAVRYLTSEMHGARPGASKAVVILV |
| | | TDVSVDSVDAAADAARSNRVTVFPIGIGDRYDAAQLRILAGPAGDSNVVK |
| | | LQRIEDLPTMVTLGNSFLHKLCSGFVRICMDEDGNEKRPGDVWTLPDQCH |
| | | TVTCQPDGQTLLKSHRVNCDRGLRPSCPNSQSPVKVEETCGCRWTCPCVC |
| | | TGSSTRHIVTFDGQNFKLTGSCSYVLFQNKEQDLEVILHNGACSPGARQG |
| | | CMKSIEVKHSALSVELHSDMEVTVNGRLVSVPYVGGNMEVNVYGAIMHEV |
| | | RFNHLGHIFTFTPQNNEFQLQLSPKTFASKTYGLCGICDENGANDFMLRD |
| | | GTVTTDWKTLVQEWTVQRPGQTCQPILEEQCLVPDSSHCQVLLLPLFAEC |
| | | HKVLAPATFYAICQQDSCHQEQVCEVIASYAHLCRTNGVCVDWRTPDFCA |
| | | MSCPPSLVYNHCEHGCPRHCDGNVSSCGDHPSEGCFCPPDKVMLEGSCVP |
| | | EEACTQCIGEDGVQHQFLEAWVPDHQPCQICTCLSGRKVNCTTQPCPTAK |
| | | APTCGLCEVARLRQNADQCCPEYECVCDPVSCDLPPVPHCERGLQPTLTN |
| | | PGECRPNFTCACRKEECKRVSPPSCPPHRLPTLRKTQCCDEYECACNCVN |
| | | STVSCPLGYLASTATNDCGCTTTTCLPDKVCVHRSTIYPVGQFWEEGCDV |
| | | CTCTDMEDAVMGLRVAQCSQKPCEDSCRSGFTYVLHEGECCGRCLPSACE |
| | | VVTGSPRGDSQSSWKSVGSQWASPENPCLINECVRVKEEVFIQQRNVSCP |
| | | QLEVPVCPSGFQLSCKTSACCPSCRCERMEACMLNGTVIGPGKTVMIDVC |
| | | TTCRCMVQVGVISGFKLECRKTTCNPCPLGYKEENNTGECCGRCLPTACT |
| | | IQLRGGQIMTLKRDETLQDGCDTHFCKVNERGEYFWEKRVTGCPPFDEHK |
| | | CLAEGGKIMKIPGTCCDTCEEPECNDITARLQYVKVGSCKSEVEVDIHYC |
| | | QGKCASKAMYSIDINDVQDQCSCCSPTRTEPMQVALHCTNGSVVYHEVLN |
| | | AMECKCSPRKCSK |
| 28 | Human VWF isoform | MGAQDEEEGIQDLDGLLVFDKIVEVTLLNLPWYNEETEGQRGEMTAPKSP |
| | 2 (UniProt: P04275- | RAKIRGTLCAEGTRGRSSTARCSLFGSDFVNTFDGSMYSFAGYCSYLLAG |
| | 2) | GCQKRSFSIIGDFQNGKRVSLSVYLGEFFDIHLFVNGTVTQGDQRVSMPY |
| | | ASKGLYLETEAGYYKLSGEAYGFVARIDGSGNFQVLLSDRYFNKTCGLCG |
| | | NFNIFAEDDFMTQEGTLTSDPYDFANSWALSSGEQWCERASPPSSSCNIS |
| | | SGEMQKEEPECNDITARLQYVKVGSCKSEVEVDIHYCQGKCASKAMYSID |
| | | INDVQDQCSCCSPTRTEPMQVALHCTNGSVVYHEVLNAMECKCSPRKCSK |
| | | I |
| 29 | Human VWF isoform | MIPARFAGVLLALALILPGTLC |
| | 1 signal peptide | |
| | (positions 1 to 22 of | |
| | UniProt: P04275-1, | |
| | v4) | |
| 30 | Human VWF isoform | AEGTRGRSSTARCSLFGSDFVNTFDGSMYSFAGYCSYLLAGGCQKRSFSI |
| | 1 lacking signal | IGDFQNGKRVSLSVYLGEFFDIHLFVNGTVTQGDQRVSMPYASKGLYLET |
| | peptide (positions 23 | EAGYYKLSGEAYGFVARIDGSGNFQVLLSDRYFNKTCGLCGNFNIFAEDD |
| | to 22 of UniProt: | FMTQEGTLTSDPYDFANSWALSSGEQWCERASPPSSSCNISSGEMQKGLW |
| | P04275-1, v4) | EQCQLLKSTSVFARCHPLVDPEPFVALCEKTLCECAGGLECACPALLEYA |
| | | RTCAQEGMVLYGWTDHSACSPVCPAGMEYRQCVSPCARTCQSLHINEMCQ |
| | | ERCVDGCSCPEGQLLDEGLCVESTECPCVHSGKRYPPGTSLSRDCNTCIC |
| | | RNSQWICSNEECPGECLVTGQSHFKSFDNRYFTFSGICQYLLARDCQDHS |
| | | FSIVIETVQCADDRDAVCTRSVTVRLPGLHNSLVKLKHGAGVAMDGQDVQ |
| | | LPLLKGDLRIQHTVTASVRLSYGEDLQMDWDGRGRLLVKLSPVYAGKTCG |
| | | LCGNYNGNQGDDFLTPSGLAEPRVEDFGNAWKLHGDCQDLQKQHSDPCAL |
| | | NPRMTRFSEEACAVLTSPTFEACHRAVSPLPYLRNCRYDVCSCSDGRECL |
| | | CGALASYAAACAGRGVRVAWREPGRCELNCPKGQVYLQCGTPCNLTCRSL |
| | | SYPDEECNEACLEGCFCPPGLYMDERGDCVPKAQCPCYYDGEIFQPEDIF |
| | | SDHHTMCYCEDGFMHCTMSGVPGSLLPDAVLSSPLSHRSKRSLSCRPPMV |
| | | KLVCPADNLRAEGLECTKTCQNYDLECMSMGCVSGCLCPPGMVRHENRCV |
| | | ALERCPCFHQGKEYAPGETVKIGCNTCVCQDRKWNCTDHVCDATCSTIGM |
| | | AHYLTFDGLKYLFPGECQYVLVQDYCGSNPGTFRILVGNKGCSHPSVKCK |
| | | KRVTILVEGGEIELFDGEVNVKRPMKDETHFEVVESGRYIILLLGKALSV |
| | | VWDRHLSISVVLKQTYQEKVCGLCGNFDGIQNNDLTSSNLQVEEDPVDFG |
| | | NSWKVSSQCADTRKVPLDSSPATCHNNIMKQTMVDSSCRILTSDVFQDCN |
| | | KLVDPEPYLDVCIYDTCSCESIGDCACFCDTIAAYAHVCAQHGKVVTWRT |
| | | ATLCPQSCEERNLRENGYECEWRYNSCAPACQVTCQHPEPLACPVQCVEG |
| | | CHAHCPPGKILDELLQTCVDPEDCPVCEVAGRRFASGKKVTLNPSDPEHC |
| | | QICHCDVVNLTCEACQEPGGLVVPPTDAPVSPTTLYVEDISEPPLHDFYC |
| | | SRLLDLVFLLDGSSRLSEAEFEVLKAFVVDMMERLRISQKWVRVAVVEYH |
| | | DGSHAYIGLKDRKRPSELRRIASQVKYAGSQVASTSEVLKYTLFQIFSKI |
| | | DRPEASRITLLLMASQEPQRMSRNFVRYVQGLKKKKVIVIPVGIGPHANL |
| | | KQIRLIEKQAPENKAFVLSSVDELEQQRDEIVSYLCDLAPEAPPPTLPPD |
| | | MAQVTVGPGLLGVSTLGPKRNSMVLDVAFVLEGSDKIGEADFNRSKEFME |
| | | EVIQRMDVGQDSIHVTVLQYSYMVTVEYPFSEAQSKGDILQRVREIRYQG |
| | | GNRTNTGLALRYLSDHSFLVSQGDREQAPNLVYMVTGNPASDEIKRLPGD |
| | | IQVVPIGVGPNANVQELERIGWPNAPILIQDFETLPREAPDLVLQRCCSG |
| | | EGLQIPTLSPAPDCSQPLDVILLLDGSSSFPASYFDEMKSFAKAFISKAN |
| | | IGPRLTQVSVLQYGSITTIDVPWNVVPEKAHLLSLVDVMQREGGPSQIGD |
| | | ALGFAVRYLTSEMHGARPGASKAVVILVTDVSVDSVDAAADAARSNRVTV |
| | | FPIGIGDRYDAAQLRILAGPAGDSNVVKLQRIEDLPTMVTLGNSFLHKLC |
| | | SGFVRICMDEDGNEKRPGDVWTLPDQCHTVTCQPDGQTLLKSHRVNCDRG |
| | | LRPSCPNSQSPVKVEETCGCRWTCPCVCTGSSTRHIVTFDGQNFKLTGSC |
| | | SYVLFQNKEQDLEVILHNGACSPGARQGCMKSIEVKHSALSVELHSDMEV |
| | | TVNGRLVSVPYVGGNMEVNVYGAIMHEVRFNHLGHIFTFTPQNNEFQLQL |
| | | SPKTFASKTYGLCGICDENGANDFMLRDGTVTTDWKTLVQEWTVQRPGQT |
| | | CQPILEEQCLVPDSSHCQVLLLPLFAECHKVLAPATFYAICQQDSCHQEQ |
| | | VCEVIASYAHLCRTNGVCVDWRTPDFCAMSCPPSLVYNHCEHGCPRHCDG |
| | | NVSSCGDHPSEGCFCPPDKVMLEGSCVPEEACTQCIGEDGVQHQFLEAWV |
| | | PDHQPCQICTCLSGRKVNCTTQPCPTAKAPTCGLCEVARLRQNADQCCPE |
| | | YECVCDPVSCDLPPVPHCERGLQPTLTNPGECRPNFTCACRKEECKRVSP |
| | | PSCPPHRLPTLRKTQCCDEYECACNCVNSTVSCPLGYLASTATNDCGCTT |
| | | TTCLPDKVCVHRSTIYPVGQFWEEGCDVCTCTDMEDAVMGLRVAQCSQKP |
| | | CEDSCRSGFTYVLHEGECCGRCLPSACEVVTGSPRGDSQSSWKSVGSQWA |
| | | SPENPCLINECVRVKEEVFIQQRNVSCPQLEVPVCPSGFQLSCKTSACCP |
| | | SCRCERMEACMLNGTVIGPGKTVMIDVCTTCRCMVQVGVISGFKLECRKT |
| | | TCNPCPLGYKEENNTGECCGRCLPTACTIQLRGGQIMTLKRDETLQDGCD |
| | | THFCKVNERGEYFWEKRVTGCPPFDEHKCLAEGGKIMKIPGTCCDTCEEP |
| | | ECNDITARLQYVKVGSCKSEVEVDIHYCQGKCASKAMYSIDINDVQDQCS |
| | | CCSPTRTEPMQVALHCTNGSVVYHEVLNAMECKCSPRKCSK |
| 31 | Human VWF antigen | AEGTRGRSSTARCSLFGSDFVNTFDGSMYSFAGYCSYLLAGGCQKRSFSI |
| | 2 | IGDFQNGKRVSLSVYLGEFFDIHLFVNGTVTQGDQRVSMPYASKGLYLET |
| | | EAGYYKLSGEAYGFVARIDGSGNFQVLLSDRYFNKTCGLCGNFNIFAEDD |
| | | FMTQEGTLTSDPYDFANSWALSSGEQWCERASPPSSSCNISSGEMQKGLW |
| | | EQCQLLKSTSVFARCHPLVDPEPFVALCEKTLCECAGGLECACPALLEYA |
| | | RTCAQEGMVLYGWTDHSACSPVCPAGMEYRQCVSPCARTCQSLHINEMCQ |
| | | ERCVDGCSCPEGQLLDEGLCVESTECPCVHSGKRYPPGTSLSRDCNTCIC |
| | | RNSQWICSNEECPGECLVTGQSHFKSFDNRYFTFSGICQYLLARDCQDHS |
| | | FSIVIETVQCADDRDAVCTRSVTVRLPGLHNSLVKLKHGAGVAMDGQDVQ |
| | | LPLLKGDLRIQHTVTASVRLSYGEDLQMDWDGRGRLLVKLSPVYAGKTCG |
| | | LCGNYNGNQGDDFLTPSGLAEPRVEDFGNAWKLHGDCQDLQKQHSDPCAL |
| | | NPRMTRFSEEACAVLTSPTFEACHRAVSPLPYLRNCRYDVCSCSDGRECL |
| | | CGALASYAAACAGRGVRVAWREPGRCELNCPKGQVYLQCGTPCNLTCRSL |
| | | SYPDEECNEACLEGCFCPPGLYMDERGDCVPKAQCPCYYDGEIFQPEDIF |
| | | SDHHTMCYCEDGFMHCTMSGVPGSLLPDAVLSSPLSHRSKR |
| 32 | Mature human VWF | SLSCRPPMVKLVCPADNLRAEGLECTKTCQNYDLECMSMGCVSGCLCPPG |
| | | MVRHENRCVALERCPCFHQGKEYAPGETVKIGCNTCVCQDRKWNCTDHVC |
| | | DATCSTIGMAHYLTFDGLKYLFPGECQYVLVQDYCGSNPGTFRILVGNKG |
| | | CSHPSVKCKKRVTILVEGGEIELFDGEVNVKRPMKDETHFEVVESGRYII |
| | | LLLGKALSVVWDRHLSISVVLKQTYQEKVCGLCGNFDGIQNNDLTSSNLQ |
| | | VEEDPVDFGNSWKVSSQCADTRKVPLDSSPATCHNNIMKQTMVDSSCRIL |
| | | TSDVFQDCNKLVDPEPYLDVCIYDTCSCESIGDCACFCDTIAAYAHVCAQ |
| | | HGKVVTWRTATLCPQSCEERNLRENGYECEWRYNSCAPACQVTCQHPEPL |
| | | ACPVQCVEGCHAHCPPGKILDELLQTCVDPEDCPVCEVAGRRFASGKKVT |
| | | LNPSDPEHCQICHCDVVNLTCEACQEPGGLVVPPTDAPVSPTTLYVEDIS |
| | | EPPLHDFYCSRLLDLVFLLDGSSRLSEAEFEVLKAFVVDMMERLRISQKW |
| | | VRVAVVEYHDGSHAYIGLKDRKRPSELRRIASQVKYAGSQVASTSEVLKY |
| | | TLFQIFSKIDRPEASRITLLLMASQEPQRMSRNFVRYVQGLKKKKVIVIP |
| | | VGIGPHANLKQIRLIEKQAPENKAFVLSSVDELEQQRDEIVSYLCDLAPE |
| | | APPPTLPPDMAQVTVGPGLLGVSTLGPKRNSMVLDVAFVLEGSDKIGEAD |
| | | FNRSKEFMEEVIQRMDVGQDSIHVTVLQYSYMVTVEYPFSEAQSKGDILQ |
| | | RVREIRYQGGNRTNTGLALRYLSDHSFLVSQGDREQAPNLVYMVTGNPAS |
| | | DEIKRLPGDIQVVPIGVGPNANVQELERIGWPNAPILIQDFETLPREAPD |
| | | LVLQRCCSGEGLQIPTLSPAPDCSQPLDVILLLDGSSSFPASYFDEMKSF |
| | | AKAFISKANIGPRLTQVSVLQYGSITTIDVPWNVVPEKAHLLSLVDVMQR |
| | | EGGPSQIGDALGFAVRYLTSEMHGARPGASKAVVILVTDVSVDSVDAAAD |
| | | AARSNRVTVFPIGIGDRYDAAQLRILAGPAGDSNVVKLQRIEDLPTMVTL |
| | | GNSFLHKLCSGFVRICMDEDGNEKRPGDVWTLPDQCHTVTCQPDGQTLLK |
| | | SHRVNCDRGLRPSCPNSQSPVKVEETCGCRWTCPCVCTGSSTRHIVTFDG |
| | | QNFKLTGSCSYVLFQNKEQDLEVILHNGACSPGARQGCMKSIEVKHSALS |
| | | VELHSDMEVTVNGRLVSVPYVGGNMEVNVYGAIMHEVRFNHLGHIFTFTP |
| | | QNNEFQLQLSPKTFASKTYGLCGICDENGANDFMLRDGTVTTDWKTLVQE |
| | | WTVQRPGQTCQPILEEQCLVPDSSHCQVLLLPLFAECHKVLAPATFYAIC |
| | | QQDSCHQEQVCEVIASYAHLCRTNGVCVDWRTPDFCAMSCPPSLVYNHCE |
| | | HGCPRHCDGNVSSCGDHPSEGCFCPPDKVMLEGSCVPEEACTQCIGEDGV |
| | | QHQFLEAWVPDHQPCQICTCLSGRKVNCTTQPCPTAKAPTCGLCEVARLR |
| | | QNADQCCPEYECVCDPVSCDLPPVPHCERGLQPTLTNPGECRPNFTCACR |
| | | KEECKRVSPPSCPPHRLPTLRKTQCCDEYECACNCVNSTVSCPLGYLAST |
| | | ATNDCGCTTTTCLPDKVCVHRSTIYPVGQFWEEGCDVCTCTDMEDAVMGL |
| | | RVAQCSQKPCEDSCRSGFTYVLHEGECCGRCLPSACEVVTGSPRGDSQSS |
| | | WKSVGSQWASPENPCLINECVRVKEEVFIQQRNVSCPQLEVPVCPSGFQL |
| | | SCKTSACCPSCRCERMEACMLNGTVIGPGKTVMIDVCTTCRCMVQVGVIS |
| | | GFKLECRKTTCNPCPLGYKEENNTGECCGRCLPTACTIQLRGGQIMTLKR |
| | | DETLQDGCDTHFCKVNERGEYFWEKRVTGCPPFDEHKCLAEGGKIMKIPG |
| | | TCCDTCEEPECNDITARLQYVKVGSCKSEVEVDIHYCQGKCASKAMYSID |
| | | INDVQDQCSCCSPTRTEPMQVALHCTNGSVVYHEVLNAMECKCSPRKCSK |
| 33 | Human VWF | RCSLFGSDFVNTFDGSMYSFAGYCSYLLAGGCQKRSFSIIGDFQNGKRVS |
| | VWFD1 | LSVYLGEFFDIHLFVNGTVTQGDQRVSMPYASKGLYLETEAGYYKLSGEA |
| | | YGFVARIDGSGNFQVLLSDRYFNKTCGLCGNFNIFAEDDFMTQEGTLTSD |
| | | PYDFANSWALSSGEQWCERASPPSSSCNISSGEMQKGLWEQCQLLKSTSV |
| | | FARCHPL |
| 34 | Human VWF TIL1 | CPAGMEYRQCVSPCARTCQSLHINEMCQERCVDGCSCPEGQLLDEGLCVE |
| | | STEC |
| 35 | Human VWF | ECLVTGQSHFKSFDNRYFTFSGICQYLLARDCQDHSFSIVIETVQCADDR |
| | VWFD2 | DAVCTRSVTVRLPGLHNSLVKLKHGAGVAMDGQDVQLPLLKGDLRIQHTV |
| | | TASVRLSYGEDLQMDWDGRGRLLVKLSPVYAGKTCGLCGNYNGNQGDDFL |
| | | TPSGLAEPRVEDFGNAWKLHGDCQDLQKQHSDPCALNPRMTRFSEEACAV |
| | | LTSPTFEACHRA |
| 36 | Human VWF TIL2 | CPKGQVYLQCGTPCNLTCRSLSYPDEECNEACLEGCFCPPGLYMDERGDC |
| | | VPKAQC |
| 37 | Human VWF TIL3 | CPADNLRAEGLECTKTCQNYDLECMSMGCVSGCLCPPGMVRHENRCVALE |
| | | RC |
| 38 | Human VWF | TCSTIGMAHYLTFDGLKYLFPGECQYVLVQDYCGSNPGTFRILVGNKGCS |
| | VWFD3 | HPSVKCKKRVTILVEGGEIELFDGEVNVKRPMKDETHFEVVESGRYIILL |
| | | LGKALSVVWDRHLSISVVLKQTYQEKVCGLCGNFDGIQNNDLTSSNLQVE |
| | | EDPVDFGNSWKVSSQCADTRKVPLDSSPATCHNNIMKQTMVDSSCRILTS |
| | | DVFQDCNKL |
| 39 | Human VWF TIL4 | YNSCAPACQVTCQHPEPLACPVQCVEGCHAHCPPGKILDELLQTCVDPED |
| | | C |
| 40 | Human VWF VWFA1 | DLVFLLDGSSRLSEAEFEVLKAFVVDMMERLRISQKWVRVAVVEYHDGSH |
| | | AYIGLKDRKRPSELRRIASQVKYAGSQVASTSEVLKYTLFQIFSKIDRPE |
| | | ASRITLLLMASQEPQRMSRNFVRYVQGLKKKKVIVIPVGIGPHANLKQIR |
| | | LIEKQAPENKAFVLSSVDELEQQRDEI |
| 41 | Human VWF VWFA2 | DVAFVLEGSDKIGEADFNRSKEFMEEVIQRMDVGQDSIHVTVLQYSYMVT |
| | | VEYPFSEAQSKGDILQRVREIRYQGGNRTNTGLALRYLSDHSFLVSQGDR |
| | | EQAPNLVYMVTGNPASDEIKRLPGDIQVVPIGVGPNANVQELERIGWPNA |
| | | PILIQDFETLPREAPDLV |
| 42 | Human VWF VWFA3 | DVILLLDGSSSFPASYFDEMKSFAKAFISKANIGPRLTQVSVLQYGSITT |
| | | IDVPWNVVPEKAHLLSLVDVMQREGGPSQIGDALGFAVRYLTSEMHGARP |
| | | GASKAVVILVTDVSVDSVDAAADAARSNRVTVFPIGIGDRYDAAQLRILA |
| | | GPAGDSNVVKLQRIEDLPTMVTLGNSFLHKL |
| 43 | Human VWF | VCTGSSTRHIVTFDGQNFKLTGSCSYVLFQNKEQDLEVILHNGACSPGAR |
| | VWFD4 | QGCMKSIEVKHSALSVELHSDMEVTVNGRLVSVPYVGGNMEVNVYGAIMH |
| | | EVRFNHLGHIFTFTPQNNEFQLQLSPKTFASKTYGLCGICDENGANDFML |
| | | RDGTVTTDWKTLVQEWTVQRPGQTCQPILEEQCLVPDSSHCQVLLLPLFA |
| | | ECHKV |
| 44 | Human VWF | TQCIGEDGVQHQFLEAWVPDHQPCQICTCLSGRKVNCTTQPCPTAKAPTC |
| | VWFC1 | GLCEVARLRQNADQCCPEYECVCD |
| 45 | Human VWF | KVCVHRSTIYPVGQFWEEGCDVCTCTDMEDAVMGLRVAQCSQKPCEDSCR |
| | VWFC2 | SGFTYVLHEGECCGRCL |
| 46 | Human VWF | EACMLNGTVIGPGKTVMIDVCTTCRCMVQVGVISGFKLECRKTTCNPCPL |
| | VWFC3 | GYKEENNTGECCGRCL |
| 47 | Human VWF isoform | CNDITARLQYVKVGSCKSEVEVDIHYCQGKCASKAMYSIDINDVQDQCSC |
| | 1, 2 CTCK | CSPTRTEPMQVALHCTNGSVVYHEVLNAMECKCSPRKCS |
| 48 | Human ADAMTS13 | VGQGTPSLVPHEEAAAPGRTTATPAGASLEWSQARGLLFSPAPQPRRLLP |
| | isoform 1 L3 region | GPQENSVQSS |
| 49 | Human ADAMTS13 | AAAPGRTTATPAGASLEWSQARGLLFSPAPQPRRLLPGP |
| | isoform 1 L3 variant | |
| | subregion | |
| 50 | Human ADAMTS13 | X₁X₂X₃X₄GRTTATX₅AGASLEWSQARGLLFSX₆AX₇QX₈RRLLX₉GX₁₀ |
| | variant L3 subregion | wherein X₁ = A, K, I, L, M or V, |
| | consensus 1 | X₂ = A, K, I, L, M or V, |
| | | X₃ = A, K, I, L, M or V, X₄ = P, K, I, L, |
| | | M or V, X₅ = P, K, I, L, M or V, |
| | | X₆ = P, K, I, L, M or V, |
| | | X₇ = P, K, I, L, M or V, X₈ = P, K, I, L, M |
| | | or V, X₉ = P, K, I, L, M or V and |
| | | X₁₀ = P, K, I, L, M or V |
| 51 | Human ADAMTS13 | VAAPGRTTATPAGASLEWSQARGLLFSPAPQPRRLLPGP |
| | A1144V L3 | |
| | subregion | |
| 52 | Human ADAMTS13 | AVAPGRTTATPAGASLEWSQARGLLFSPAPQPRRLLPGP |
| | A1145V L3 | |
| | subregion | |
| 53 | Human ADAMTS13 | AAVPGRTTATPAGASLEWSQARGLLFSPAPQPRRLLPGP |
| | A1146V L3 | |
| | subregion | |
| 54 | Human ADAMTS13 | AAAVGRTTATPAGASLEWSQARGLLFSPAPQPRRLLPGP |
| | P1147V L3 | |
| | subregion | |
| 55 | Human ADAMTS13 | AAAPGRTTATVAGASLEWSQARGLLFSPAPQPRRLLPGP |
| | P1154V L3 | |
| | subregion | |
| 56 | Human ADAMTS13 | AAAPGRTTATPAGASLEWSQARGLLFSVAPQPRRLLPGP |
| | P1171V L3 | |
| | subregion | |
| 57 | Human ADAMTS13 | AAAPGRTTATPAGASLEWSQARGLLFSPAVQPRRLLPGP |
| | P1173V L3 | |
| | subregion | |
| 58 | Human ADAMTS13 | AAAPGRTTATPAGASLEWSQARGLLFSPAPQVRRLLPGP |
| | P1175V L3 | |
| | subregion | |
| 59 | Human ADAMTS13 | AAAPGRTTATPAGASLEWSQARGLLFSPAPQPRRLLVGP |
| | P1180V L3 | |
| | subregion | |
| 60 | Human ADAMTS13 | AAAPGRTTATPAGASLEWSQARGLLFSPAPQPRRLLPGV |
| | P1182V L3 | |
| | subregion | |
| 61 | Mature ADAMTS13 | AAGGILHLELLVAVGPDVFQAHQEDTERYVLTNLNIGAELLRDPSLGAQF |
| | A1144V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEVAAPGRTTATPAGASLEWSQARGLLFSPAPQ |
| | | PRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 62 | Mature ADAMTS13 | AAGGILHLELLVAVGPDVFQAHQEDTERYVLTNLNIGAELLRDPSLGAQF |
| | A1145V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAVAPGRTTATPAGASLEWSQARGLLFSPAPQ |
| | | PRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 63 | Mature ADAMTS13 | AAGGILHLELLVAVGPDVFQAHQEDTERYVLTNLNIGAELLRDPSLGAQF |
| | A1146V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAVPGRTTATPAGASLEWSQARGLLFSPAPQ |
| | | PRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 64 | Mature ADAMTS13 | AAGGILHLELLVAVGPDVFQAHQEDTERYVLTNLNIGAELLRDPSLGAQF |
| | P1147V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAAVGRTTATPAGASLEWSQARGLLFSPAPQ |
| | | PRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 65 | Mature ADAMTS13 | AAGGILHLELLVAVGPDVFQAHQEDTERYVLTNLNIGAELLRDPSLGAQF |
| | P1154V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAAPGRTTATVAGASLEWSQARGLLFSPAPQ |
| | | PRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 66 | Mature ADAMTS13 | AAGGILHLELLVAVGPDVFQAHQEDTERYVLTNLNIGAELLRDPSLGAQF |
| | P1171V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAAPGRTTATPAGASLEWSQARGLLFSVAPQ |
| | | PRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 67 | Mature ADAMTS13 | AAGGILHLELLVAVGPDVFQAHQEDTERYVLTNLNIGAELLRDPSLGAQF |
| | P1173V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAAPGRTTATPAGASLEWSQARGLLFSPAVQ |
| | | PRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 68 | Mature ADAMTS13 | AAGGILHLELLVAVGPDVFQAHQEDTERYVLTNLNIGAELLRDPSLGAQF |
| | P1175V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAAPGRTTATPAGASLEWSQARGLLFSPAPQ |
| | | VRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 69 | Mature ADAMTS13 | AAGGILHLELLVAVGPDVFQAHQEDTERYVLTNLNIGAELLRDPSLGAQF |
| | P1180V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAAPGRTTATPAGASLEWSQARGLLFSPAPQ |
| | | PRRLLVGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 70 | Mature ADAMTS13 | AAGGILHLELLVAVGPDVFQAHQEDTERYVLTNLNIGAELLRDPSLGAQF |
| | P1182V | RVHLVKMVILTEPEGAPNITANLTSSLLSVCGWSQTINPEDDTDPGHADL |
| | | VLYITRFDLELPDGNRQVRGVTQLGGACSPTWSCLITEDTGFDLGVTIAH |
| | | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAREYVTFL |
| | | TVTPNLTSVYIANHRPLFTHLAVRIGGRYVVAGKMSISPNTTYPSLLEDG |
| | | RVEYRVALTEDRLPRLEEIRIWGPLQEDADIQVYRRYGEEYGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAAPGRTTATPAGASLEWSQARGLLFSPAPQ |
| | | PRRLLPGVQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 71 | Mature ADAMTS13 | AAGGILHLELLVAVGPDVFQAHQEDTERYVLTNLNIGAELLRDPSLGAQF |
| | R568K/F592Y/R660 | RVHLVKMVILTEPEGAPNITANLTSSLLSVCGWSQTINPEDDTDPGHADL |
| | K/Y661F/Y665F | VLYITRFDLELPDGNRQVRGVTQLGGACSPTWSCLITEDTGFDLGVTIAH |
| | variant | EIGHSFGLEHDGAPGSGCGPSGHVMASDGAAPRAGLAWSPCSRRQLLSLL |
| | | SAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSL |
| | | VELTPIAAVHGRWSSWGPRSPCSRSCGGGVVTRRRQCNNPRPAFGGRACV |
| | | GADLQAEMCNTQACEKTQLEFMSQQCARTDGQPLRSSPGGASFYHWGAAV |
| | | PHSQGDALCRHMCRAIGESFIMKRGDSFLDGTRCMPSGPREDGTLSLCVS |
| | | GSCRTFGCDGRMDSQQVWDRCQVCGGDNSTCSPRKGSFTAGRAKEYVTFL |
| | | TVTPNLTSVYIANHRPLYTHLAVRIGGRYVVAGKMSISPNTTYPSLLEDG |
| | | RVEYRVALTEDRLPRLEEIRIWGPLQEDADIQVYRKFGEEFGNLTRPDIT |
| | | FTYFQPKPRQAWVWAAVRGPCSVSCGAGLRWVNYSCLDQARKELVETVQC |
| | | QGSQQPPAWPEACVLEPCPPYWAVGDFGPCSASCGGGLRERPVRCVEAQG |
| | | SLLKTLPPARCRAGAQQPAVALETCNPQPCPARWEVSEPSSCTSAGGAGL |
| | | ALENETCVPGADGLEAPVTEGPGSVDEKLPAPEPCVGMSCPPGWGHLDAT |
| | | SAGEKAPSPWGSIRTGAQAAHVWTPAAGSCSVSCGRGLMELRFLCMDSAL |
| | | RVPVQEELCGLASKPGSRREVCQAVPCPARWQYKLAACSVSCGRGVVRRI |
| | | LYCARAHGEDDGEEILLDTQCQGLPRPEPQEACSLEPCPPRWKVMSLGPC |
| | | SASCGLGTARRSVACVQLDQGQDVEVDEAACAALVRPEASVPCLIADCTY |
| | | RWHVGTWMECSVSCGDGIQRRRDTCLGPQAQAPVPADFCQHLPKPVTVRG |
| | | CWAGPCVGQGTPSLVPHEEAAAPGRTTATPAGASLEWSQARGLLFSPAPQ |
| | | PRRLLPGPQENSVQSSACGRQHLEPTGTIDMRGPGQADCAVAIGRPLGEV |
| | | VTLRVLESSLNCSAGDMLLLWGRLTWRKMCRKLLDMTFSSKTNTLVVRQR |
| | | CGRPGGGVLLRYGSQLAPETFYRECDMQLFGPWGEIVSPSLSPATSNAGG |
| | | CRLFINVAPHARIAIHALATNMGAGTEGANASYILIRDTHSLRTTAFHGQ |
| | | QVLYWESESSQAEMEFSEGFLKAQASLRGQYWTLQSWVPEMQDPQSWKGK |
| | | EGT |
| 72 | Exemplified wildtype | MHQRHPRARCPPLCVAGILACGFLLGCWGPSHFQQSCLQALEPQAVSSYL |
| | ADAMTS13 | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 73 | Exemplified | MHQRHPRARCPPLCVAGILACGFLLGCWGPSHFQQSCLQALEPQAVSSYL |
| | ADAMTS13 | SPGAPLKGRPPSPGFQRQRQRQRRAAGGILHLELLVAVGPDVFQAHQEDT |
| | R568K/F592Y/R660 | ERYVLTNLNIGAELLRDPSLGAQFRVHLVKMVILTEPEGAPNITANLTSS |
| | K/Y661F/Y665F | LLSVCGWSQTINPEDDTDPGHADLVLYITRFDLELPDGNRQVRGVTQLGG |
| | variant | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAKEYVTFLTVTPNLTSVYIANHRPLYTHLAVRIG |
| | | GRYVVAGKMSISPNTTYPSLLEDGRVEYRVALTEDRLPRLEEIRIWGPLQ |
| | | EDADIQVYRKFGEEFGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 74 | Exemplified A1144V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEVAAPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 75 | Exemplified A1145V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAVAPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 76 | Exemplified A1146V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAVPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 77 | Exemplified P1147V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAVGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 78 | Exemplified P1154V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATVAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 79 | Exemplified P1171V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRGRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSVAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 80 | Exemplified P1173V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAVQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 81 | Exemplified P1175V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQVRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 82 | Exemplified P1180V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQPRRLLVGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 83 | Exemplified P1182V | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQPRRLLPGVQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 84 | Exemplified wildtype | ATGCACCAGCGTCACCCCCGGGCAAGATGCCCTCCCCTCTGTGTGGCCGG |
| | ADAMTS13 | AATCCTTGCCTGTGGCTTTCTCCTGGGCTGCTGGGGACCCTCCCATTTCC |
| | nucleotide sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGG |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGGAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 85 | Exemplified | ATGCACCAGCGTCACCCCCGGGCAAGATGCCCTCCCCTCTGTGTGGCCGG |
| | ADAMTS13 | AATCCTTGCCTGTGGCTTTCTCCTGGGCTGCTGGGGACCCTCCCATTTCC |
| | R568K/F592Y/R660 | AGCAGAGCTGTCTTCAGGCTTTGGAGCCACAGGCCGTGTCTTCTTACTTG |
| | K/Y661F/Y665F | AGCCCTGGTGCTCCCTTAAAAGGCCGCCCTCCTTCCCCTGGCTTCCAGAG |
| | variant nucleotide | GCAGAGGCAGAGGCAGAGGGGGGCTGCAGGCGGCATCCTACACCTGGAGC |
| | sequence | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAAGGAATATGTCACGTTTCTGACAGTTACCCCCAACCTGACCAGTGTCT |
| | | ACATTGCCAACCACAGGCCTCTCTACACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGAAGTTTGGCGAGGAGTTTGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGGGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGGAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGGGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 86 | Exemplified A1144V | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGGGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGTGGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGGAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 87 | Exemplified A1145V | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGGTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGTGGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGCGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 88 | Exemplified A1146V | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGTGCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 89 | Exemplified P1147V | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGCGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCAGGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTGTGGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGGAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGCGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 90 | Exemplified P1154V | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGCGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGGAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCGTGGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGGGGCCCCTCGGGGAGGTGGTGACCCTCCGCGTCCTTGAGAGTTCTC |
| | | TCAACTGGAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGCGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 91 | variant nucleotide | ATGCACCAGCGTCACCCCCGGGCAAGATGCCCTCCCCTCTGTGTGGCCGG |
| | sequence | AATCCTTGCCTGTGGCTTTCTCCTGGGCTGCTGGGGACCCTCCCATTTCC |
| | Exemplified P1171V | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGCCCTG |
| | | AGCCTGTGTGTGTCGGGCAGCTGCAGGACATTTGGCTGTGATGGTAGGAT |
| | | GGAGAGACAGCTTCCTCGATGGGACCCGGTGTATGCCAAGTGGCCCCCGG |
| | | GAGGACGGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCGTGGCTCCCCAGCCTCGGGGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 92 | variant nucleotide | ATGCACCAGCGTCACCCCCGGGCAAGATGCCCTCCCCTCTGTGTGGCCGG |
| | sequence | AATCCTTGCCTGTGGCTTTCTCCTGGGCTGCTGGGGACCCTCCCATTTCC |
| | Exemplified P1173V | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGCTCCC |
| | | AGCAGGTATGGGACAGGTGCCAGGTGTGTGGTGGGGACAACAGCACGTGC |
| | | AGGAGACAGCTTCCTCGATGGGACCCGGTGTATGCCAAGTGGCCCCCGGG |
| | | AGGACGGGACCCTGAGCCTGTGTGTGTCGGGCAGCTGCAGGACATTTGGC |
| | | TGTGATGGTAGGATGGACCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGGTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTGTGCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGGAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 93 | Exemplified P1175V | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGGAGCGCCTCCTGTGGGGGTGGCCTGGGGGAGGGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGGTGCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 94 | Exemplified P1180V | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGGTGGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 95 | Exemplified P1182V | ATGCACCAGCGTCACCCCCGGGCAAGATGCCCTCCCCTCTGTGTGGCC |
| | variant nucleotide | GGAATCCTTGCCTGTGGCTTTCTCCTGGGCTGCTGGGGACCCTCCCATTT |
| | sequence | CCAGCAGAGCTGTCTTCAGGCTTTGGAGCCACAGGCCGTGTCTTCTTACT |
| | | TGAGCCCTGGTGCTCCCTTAAAAGGCCGCCCTCCTTCCCCTGGCTTCCAG |
| | | AGGCAGAGGCAGAGGCAGAGGCGGGCTGCAGGCGGCATCCTACACCTGGA |
| | | GCTGCTGGTGGCCGTGGGCCCCGATGTCTTCCAGGCTCACCAGGAGGACA |
| | | CAGAGCGCTATGTGCTCACCAACCTCAACATCGGGGCAGAACTGCTTCGG |
| | | GACCCGTCCCTGGGGGCTCAGTTTCGGGTGCACCTGGTGAAGATGGTCAT |
| | | TCTGACAGAGCCTGAGGGTGCTCCAAATATCACAGCCAACCTCACCTCGT |
| | | CCCTGCTGAGCGTCTGTGGGTGGAGCCAGACCATCAACCCTGAGGACGAC |
| | | ACGGATCCTGGCCATGCTGACCTGGTCCTCTATATCACTAGGTTTGACCT |
| | | GGAGTTGCCTGATGGTAACCGGCAGGTGCGGGGCGTCACCCAGCTGGGCG |
| | | GTGCCTGCTCCCCAACCTGGAGCTGCCTCATTACCGAGGACACTGGCTTC |
| | | GACCTGGGAGTCACCATTGCCCATGAGATTGGGCACAGCTTCGGCCTGGA |
| | | GCACGACGGCGCGCCCGGCAGCGGCTGCGGCCCCAGCGGACACGTGATGG |
| | | CTTCGGACGGCGCCGCGCCCCGCGCCGGCCTCGCCTGGTCCCCCTGCAGC |
| | | CGCCGGCAGCTGCTGAGCCTGCTCAGCGCAGGACGGGCGCGCTGCGTGTG |
| | | GGACCCGCCGCGGCCTCAACCCGGGTCCGCGGGGCACCCGCCGGATGCGC |
| | | AGCCTGGCCTCTACTACAGCGCCAACGAGCAGTGCCGCGTGGCCTTCGGC |
| | | CCCAAGGCTGTCGCCTGCACCTTCGCCAGGGAGCACCTGGATATGTGCCA |
| | | GGCCCTCTCCTGCCACACAGACCCGCTGGACCAAAGCAGCTGCAGCCGCC |
| | | TCCTCGTTCCTCTCCTGGATGGGACAGAATGTGGCGTGGAGAAGTGGTGC |
| | | TCCAAAGGTCGCTGCCGCTCCCTGGTGGAGCTGACCCCCATAGCAGCAGT |
| | | GCATGGGCGCTGGTCTAGCTGGGGTCCCCGAAGTCCTTGCTCCCGCTCCT |
| | | GCGGAGGAGGTGTGGTCACCAGGAGGCGGCAGTGCAACAACCCCAGACCT |
| | | GCCTTTGGGGGGCGTGCATGTGTTGGTGCTGACCTCCAGGCCGAGATGTG |
| | | CAACACTCAGGCCTGCGAGAAGACCCAGCTGGAGTTCATGTCGCAACAGT |
| | | GCGCCAGGACCGACGGCCAGCCGCTGCGCTCCTCCCCTGGGGGCGCCTCC |
| | | TTCTACCACTGGGGTGCTGCTGTACCACACAGCCAAGGGGATGCTCTGTG |
| | | CAGACACATGTGCCGGGCCATTGGCGAGAGCTTCATCATGAAGCGTGGAG |
| | | ACAGCTTCCTCGATGGGACCCGGTGTATGCCAAGTGGCCCCCGGGAGGAC |
| | | GGGACCCTGAGCCTGTGTGTGTCGGGCAGCTGCAGGACATTTGGCTGTGA |
| | | TGGTAGGATGGACTCCCAGCAGGTATGGGACAGGTGCCAGGTGTGTGGTG |
| | | GGGACAACAGCACGTGCAGCCCACGGAAGGGCTCTTTCACAGCTGGCAGA |
| | | GCGAGAGAATATGTCACGTTTCTGACAGTTACCCCCAACCTGACCAGTGT |
| | | CTACATTGCCAACCACAGGCCTCTCTTCACACACTTGGCGGTGAGGATCG |
| | | GAGGGCGCTATGTCGTGGCTGGGAAGATGAGCATCTCCCCTAACACCACC |
| | | TACCCCTCCCTCCTGGAGGATGGTCGTGTCGAGTACAGAGTGGCCCTCAC |
| | | CGAGGACCGGCTGCCCCGCCTGGAGGAGATCCGCATCTGGGGACCCCTCC |
| | | AGGAAGATGCTGACATCCAGGTTTACAGGCGGTATGGCGAGGAGTATGGC |
| | | AACCTCACCCGCCCAGACATCACCTTCACCTACTTCCAGCCTAAGCCACG |
| | | GCAGGCCTGGGTGTGGGCCGCTGTGCGTGGGCCCTGCTCGGTGAGCTGTG |
| | | GGGCAGGGCTGCGCTGGGTAAACTACAGCTGCCTGGACCAGGCCAGGAAG |
| | | GAGTTGGTGGAGACTGTCCAGTGCCAAGGGAGCCAGCAGCCACCAGCGTG |
| | | GCCAGAGGCCTGCGTGCTCGAACCCTGCCCTCCCTACTGGGCGGTGGGAG |
| | | ACTTCGGCCCATGCAGCGCCTCCTGTGGGGGTGGCCTGCGGGAGCGGCCA |
| | | GTGCGCTGCGTGGAGGCCCAGGGCAGCCTCCTGAAGACATTGCCCCCAGC |
| | | CCGGTGCAGAGCAGGGGCCCAGCAGCCAGCTGTGGCGCTGGAAACCTGCA |
| | | ACCCCCAGCCCTGCCCTGCCAGGTGGGAGGTGTCAGAGCCCAGCTCATGC |
| | | ACATCAGCTGGTGGAGCAGGCCTGGCCTTGGAGAACGAGACCTGTGTGCC |
| | | AGGGGCAGATGGCCTGGAGGCTCCAGTGACTGAGGGGCCTGGCTCCGTAG |
| | | ATGAGAAGCTGCCTGCCCCTGAGCCCTGTGTCGGGATGTCATGTCCTCCA |
| | | GGCTGGGGCCATCTGGATGCCACCTCTGCAGGGGAGAAGGCTCCCTCCCC |
| | | ATGGGGCAGCATCAGGACGGGGGCTCAAGCTGCACACGTGTGGACCCCTG |
| | | CGGCAGGGTCGTGCTCCGTCTCCTGCGGGCGAGGTCTGATGGAGCTCCGT |
| | | TTCCTGTGCATGGACTCTGCCCTCAGGGTGCCTGTCCAGGAAGAGCTGTG |
| | | TGGCCTGGCAAGCAAGCCTGGGAGCCGGGGGGAGGTCTGCCAGGCTGTCC |
| | | CGTGCCCTGCTCGGTGGCAGTACAAGCTGGCGGCCTGCAGCGTGAGCTGT |
| | | GGGAGAGGGGTCGTGCGGAGGATCCTGTATTGTGCCCGGGCCCATGGGGA |
| | | GGACGATGGTGAGGAGATCCTGTTGGACACCCAGTGCCAGGGGCTGCCTC |
| | | GCCCGGAACCCCAGGAGGCCTGCAGCCTGGAGCCCTGCCCACCTAGGTGG |
| | | AAAGTCATGTCCCTTGGCCCATGTTCGGCCAGCTGTGGCCTTGGCACTGC |
| | | TAGACGCTCGGTGGCCTGTGTGCAGCTCGACCAAGGCCAGGACGTGGAGG |
| | | TGGACGAGGCGGCCTGTGCGGCGCTGGTGCGGCCCGAGGCCAGTGTCCCC |
| | | TGTCTCATTGCCGACTGCACCTACCGCTGGCATGTTGGCACCTGGATGGA |
| | | GTGCTCTGTTTCCTGTGGGGATGGCATCCAGCGCCGGCGTGACACCTGCC |
| | | TCGGACCCCAGGCCCAGGCGCCTGTGCCAGCTGATTTCTGCCAGCACTTG |
| | | CCCAAGCCGGTGACTGTGCGTGGCTGCTGGGCTGGGCCCTGTGTGGGACA |
| | | GGGTACGCCCAGCCTGGTGCCCCACGAAGAAGCCGCTGCTCCAGGACGGA |
| | | CCACAGCCACCCCTGCTGGTGCCTCCCTGGAGTGGTCCCAGGCCCGGGGC |
| | | CTGCTCTTCTCCCCGGCTCCCCAGCCTCGGCGGCTCCTGCCCGGGGTGCA |
| | | GGAAAACTCAGTGCAGTCCAGTGCCTGTGGCAGGCAGCACCTTGAGCCAA |
| | | CAGGAACCATTGACATGCGAGGCCCAGGGCAGGCAGACTGTGCAGTGGCC |
| | | ATTGGGCGGCCCCTCGGGGAGGTGGTGACCCTCCGCGTCCTTGAGAGTTC |
| | | TCTCAACTGCAGTGCGGGGGACATGTTGCTGCTTTGGGGCCGGCTCACCT |
| | | GGAGGAAGATGTGCAGGAAGCTGTTGGACATGACTTTCAGCTCCAAGACC |
| | | AACACGCTGGTGGTGAGGCAGCGCTGCGGGCGGCCAGGAGGTGGGGTGCT |
| | | GCTGCGGTATGGGAGCCAGCTTGCTCCTGAAACCTTCTACAGAGAATGTG |
| | | ACATGCAGCTCTTTGGGCCCTGGGGTGAAATCGTGAGCCCCTCGCTGAGT |
| | | CCAGCCACGAGTAATGCAGGGGGCTGCCGGCTCTTCATTAATGTGGCTCC |
| | | GCACGCACGGATTGCCATCCATGCCCTGGCCACCAACATGGGCGCTGGGA |
| | | CCGAGGGAGCCAATGCCAGCTACATCTTGATCCGGGACACCCACAGCTTG |
| | | AGGACCACAGCGTTCCATGGGCAGCAGGTGCTCTACTGGGAGTCAGAGAG |
| | | CAGCCAGGCTGAGATGGAGTTCAGCGAGGGCTTCCTGAAGGCTCAGGCCA |
| | | GCCTGCGGGGCCAGTACTGGACCCTCCAATCATGGGTACCGGAGATGCAG |
| | | GACCCTCAGTCCTGGAAGGGAAAGGAAGGAACCTCTAGAGGGCCCTTCGA |
| | | ACAAAAACTCATCTCAGAAGAGGATCTGAATATGCATACCGGTCATCATC |
| | | ACCATCACCAT |
| 96 | Exemplified A1144K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEKAAPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 97 | Exemplified A11441 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEIAAPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 98 | Exemplified A1145K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVE |
| | | TVQCQGSQQPPAWPEACVLEPCPPYWAVGDFGPCSASCGGGLRERPVRCV |
| | | EAQGSLLKTLPPARCRAGAQQPAVALETCNPQPCPARWEVSEPSSCTSAG |
| | | GAGLALENETCVPGADGLEAPVTEGPGSVDEKLPAPEPCVGMSCPPGWGH |
| | | LDATSAGEKAPSPWGSIRTGAQAAHVWTPAAGSCSVSCGRGLMELRFLCM |
| | | DSALRVPVQEELCGLASKPGSRREVCQAVPCPARWQYKLAACSVSCGRGV |
| | | VRRILYCARAHGEDDGEEILLDTQCQGLPRPEPQEACSLEPCPPRWKVMS |
| | | LGPCSASCGLGTARRSVACVQLDQGQDVEVDEAACAALVRPEASVPCLIA |
| | | DCTYRWHVGTWMECSVSCGDGIQRRRDTCLGPQAQAPVPADFCQHLPKPV |
| | | TVRGCWAGPCVGQGTPSLVPHEEAKAPGRTTATPAGASLEWSQARGLLFS |
| | | PAPQPRRLLPGPQENSVQSSACGRQHLEPTGTIDMRGPGQADCAVAIGRP |
| | | LGEVVTLRVLESSLNCSAGDMLLLWGRLTWRKMCRKLLDMTFSSKTNTLV |
| | | VRQRCGRPGGGVLLRYGSQLAPETFYRECDMQLFGPWGEIVSPSLSPATS |
| | | NAGGCRLFINVAPHARIAIHALATNMGAGTEGANASYILIRDTHSLRTTA |
| | | FHGQQVLYWESESSQAEMEFSEGFLKAQASLRGQYWTLQSWVPEMQDPQS |
| | | WKGKEGTSRGPFEQKLISEEDLNMHTGHHHHHH |
| 99 | Exemplified A11451 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAIAPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 100 | Exemplified A1146K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAKPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 101 | Exemplified A11461 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAIPGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 102 | Exemplified P1147K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAKGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 103 | Exemplified P11471 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAIGRT |
| | | TATPAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 104 | Exemplified P1154K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASGGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATKAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 105 | Exemplified P11541 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATIAGASLEWSQARGLLFSPAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 106 | Exemplified P1171K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSKAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 107 | Exemplified P11711 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSIAPQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 108 | Exemplified P1173K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAKQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 109 | Exemplified P11731 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAIQPRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 110 | Exemplified P1175K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQKRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 111 | Exemplified P11751 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQIRRLLPGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 112 | Exemplified P1180K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQPRRLLKGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 113 | Exemplified P11801 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQPRRLLIGPQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 114 | Exemplified P1182K | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQPRRLLPGKQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 115 | Exemplified P11821 | MHQRHPRARCPPLCVAGILACGFLLGCWGPSHFQQSCLQALEPQAVSSYL |
| | variant | SPGAPLKGRPPSPGFQRQRQRQRRAAGGILHLELLVAVGPDVFQAHQEDT |
| | | ERYVLTNLNIGAELLRDPSLGAQFRVHLVKMVILTEPEGAPNITANLTSS |
| | | LLSVCGWSQTINPEDDTDPGHADLVLYITRFDLELPDGNRQVRGVTQLGG |
| | | ACSPTWSCLITEDTGFDLGVTIAHEIGHSFGLEHDGAPGSGCGPSGHVMA |
| | | SDGAAPRAGLAWSPCSRRQLLSLLSAGRARCVWDPPRPQPGSAGHPPDAQ |
| | | PGLYYSANEQCRVAFGPKAVACTFAREHLDMCQALSCHTDPLDQSSCSRL |
| | | LVPLLDGTECGVEKWCSKGRCRSLVELTPIAAVHGRWSSWGPRSPCSRSC |
| | | GGGVVTRRRQCNNPRPAFGGRACVGADLQAEMCNTQACEKTQLEFMSQQC |
| | | ARTDGQPLRSSPGGASFYHWGAAVPHSQGDALCRHMCRAIGESFIMKRGD |
| | | SFLDGTRCMPSGPREDGTLSLCVSGSCRTFGCDGRMDSQQVWDRCQVCGG |
| | | DNSTCSPRKGSFTAGRAREYVTFLTVTPNLTSVYIANHRPLFTHLAVRIG |
| | | GRYVVAGKMSISPNTTYPSLLEDGRVEYRVALTEDRLPRLEEIRIWGPLQ |
| | | EDADIQVYRRYGEEYGNLTRPDITFTYFQPKPRQAWVWAAVRGPCSVSCG |
| | | AGLRWVNYSCLDQARKELVETVQCQGSQQPPAWPEACVLEPCPPYWAVGD |
| | | FGPCSASCGGGLRERPVRCVEAQGSLLKTLPPARCRAGAQQPAVALETCN |
| | | PQPCPARWEVSEPSSCTSAGGAGLALENETCVPGADGLEAPVTEGPGSVD |
| | | EKLPAPEPCVGMSCPPGWGHLDATSAGEKAPSPWGSIRTGAQAAHVWTPA |
| | | AGSCSVSCGRGLMELRFLCMDSALRVPVQEELCGLASKPGSRREVCQAVP |
| | | CPARWQYKLAACSVSCGRGVVRRILYCARAHGEDDGEEILLDTQCQGLPR |
| | | PEPQEACSLEPCPPRWKVMSLGPCSASCGLGTARRSVACVQLDQGQDVEV |
| | | DEAACAALVRPEASVPCLIADCTYRWHVGTWMECSVSCGDGIQRRRDTCL |
| | | GPQAQAPVPADFCQHLPKPVTVRGCWAGPCVGQGTPSLVPHEEAAAPGRT |
| | | TATPAGASLEWSQARGLLFSPAPQPRRLLPGIQENSVQSSACGRQHLEPT |
| | | GTIDMRGPGQADCAVAIGRPLGEVVTLRVLESSLNCSAGDMLLLWGRLTW |
| | | RKMCRKLLDMTFSSKTNTLVVRQRCGRPGGGVLLRYGSQLAPETFYRECD |
| | | MQLFGPWGEIVSPSLSPATSNAGGCRLFINVAPHARIAIHALATNMGAGT |
| | | EGANASYILIRDTHSLRTTAFHGQQVLYWESESSQAEMEFSEGFLKAQAS |
| | | LRGQYWTLQSWVPEMQDPQSWKGKEGTSRGPFEQKLISEEDLNMHTGHHH |
| | | HHH |
| 116 | Exemplified A1144K | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGGTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGGGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAAAGGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGGAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 117 | Exemplified A11441 | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGGGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGGGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAATTGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGGGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGGGCTGGGACC |
| | | GAGGGAGCCAATGCCAGCTACATCTTGATCCGGGACACCCACAGCTTGAG |
| | | GACCACAGGGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 118 | Exemplified A1145K | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCAAGGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGCGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 119 | Exemplified A11451 | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGCGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCCCCTACTGGGCGGTGGGAGACT |
| | | TCGGCCCATGCAGCGCCTCCTGTGGGGGTGGCCTGGGGGAGCGGCCAGTG |
| | | CGCTGCGTGGAGGCCCAGGGCAGCCTCCTGAAGACATTGCCCCCAGCCCG |
| | | GTGCAGAGCAGGGGCCCAGCAGCCAGCTGTGGCGCTGGAAACCTGCAACC |
| | | CCCAGCCCTGCCCTGCCAGGTGGGAGGTGTCAGAGCCCAGCTCATGCACA |
| | | TCAGCTGGTGGAGCAGGCCTGGCCTTGGAGAACGAGACCTGTGTGCCAGG |
| | | GGCAGATGGCCTGGAGGCTCCAGTGACTGAGGGGCCTGGCTCCGTAGATG |
| | | AGAAGCTGCCTGCCCCTGAGCCCTGTGTCGGGATGTCATGTCCTCCAGGC |
| | | TGGGGCCATCTGGATGCCACCTCTGCAGGGGAGAAGGCTCCCTCCCCATG |
| | | GGGCAGCATCAGGACGGGGGCTCAAGCTGCACACGTGTGGACCCCTGCGG |
| | | CAGGGTCGTGCTCCGTCTCCTGCGGGCGAGGTCTGATGGAGCTCCGTTTC |
| | | CTGTGCATGGACTCTGCCCTCAGGGTGCCTGTCCAGGAAGAGCTGTGTGG |
| | | CCTGGCAAGCAAGCCTGGGAGCCGGGGGGAGGTCTGCCAGGCTGTCCCGT |
| | | GCCCTGCTCGGTGGCAGTACAAGCTGGCGGCCTGCAGCGTGAGCTGTGGG |
| | | AGAGGGGTCGTGCGGAGGATCCTGTATTGTGCCCGGGCCCATGGGGAGGA |
| | | CGATGGTGAGGAGATCCTGTTGGACACCCAGTGCCAGGGGCTGCCTCGCC |
| | | CGGAACCCCAGGAGGCCTGCAGCCTGGAGCCCTGCCCACCTAGGTGGAAA |
| | | GTCATGTCCCTTGGCCCATGTTCGGCCAGCTGTGGCCTTGGCACTGCTAG |
| | | ACGCTCGGTGGCCTGTGTGCAGCTCGACCAAGGCCAGGACGTGGAGGTGG |
| | | ACGAGGCGGCCTGTGCGGCGCTGGTGCGGCCCGAGGCCAGTGTCCCCTGT |
| | | CTCATTGCCGACTGCACCTACCGCTGGCATGTTGGCACCTGGATGGAGTG |
| | | CTCTGTTTCCTGTGGGGATGGCATCCAGCGCCGGCGTGACACCTGCCTCG |
| | | GACCCCAGGCCCAGGCGCCTGTGCCAGCTGATTTCTGCCAGCACTTGCCC |
| | | AAGCCGGTGACTGTGCGTGGCTGCTGGGCTGGGCCCTGTGTGGGACAGGG |
| | | TACGCCCAGCCTGGTGCCCCACGAAGAAGCCATTGCTCCAGGACGGACCA |
| | | CAGCCACCCCTGCTGGTGCCTCCCTGGAGTGGTCCCAGGCCCGGGGCCTG |
| | | CTCTTCTCCCCGGCTCCCCAGCCTCGGCGGCTCCTGCCCGGGCCCCAGGA |
| | | AAACTCAGTGCAGTCCAGTGCCTGTGGCAGGCAGCACCTTGAGCCAACAG |
| | | GAACCATTGACATGCGAGGCCCAGGGCAGGCAGACTGTGCAGTGGCCATT |
| | | GGGGGGCCCCTCGGGGAGGTGGTGACCCTCCGCGTCCTTGAGAGTTCTCT |
| | | CAACTGCAGTGCGGGGGACATGTTGCTGCTTTGGGGCCGGCTCACCTGGA |
| | | GGAAGATGTGCAGGAAGCTGTTGGACATGACTTTCAGCTCCAAGACCAAC |
| | | ACGCTGGTGGTGAGGCAGCGCTGCGGGCGGCCAGGAGGTGGGGTGCTGCT |
| | | GCGGTATGGGAGCCAGCTTGCTCCTGAAACCTTCTACAGAGAATGTGACA |
| | | TGCAGCTCTTTGGGCCCTGGGGTGAAATCGTGAGCCCCTCGCTGAGTCCA |
| | | GCCACGAGTAATGCAGGGGGCTGCCGGCTCTTCATTAATGTGGCTCCGCA |
| | | CGCACGGATTGCCATCCATGCCCTGGCCACCAACATGGGCGCTGGGACCG |
| | | AGGGAGCCAATGCCAGCTACATCTTGATCCGGGACACCCACAGCTTGAGG |
| | | ACCACAGCGTTCCATGGGCAGCAGGTGCTCTACTGGGAGTCAGAGAGCAG |
| | | CCAGGCTGAGATGGAGTTCAGCGAGGGCTTCCTGAAGGCTCAGGCCAGCC |
| | | TGCGGGGCCAGTACTGGACCCTCCAATCATGGGTACCGGAGATGCAGGAC |
| | | CCTCAGTCCTGGAAGGGAAAGGAAGGAACCTCTAGAGGGCCCTTCGAACA |
| | | AAAACTCATCTCAGAAGAGGATCTGAATATGCATACCGGTCATCATCACC |
| | | ATCACCAT |
| 120 | Exemplified A1146K | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTAAGCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGGAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 121 | Exemplified A11461 | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGG |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGGTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTATTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGGGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGGGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGGGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 122 | Exemplified P1147K | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGGA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTAAGGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGGAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 123 | Exemplified P11471 | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGGTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTATTGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGCGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 124 | Exemplified P1154K | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCC |
| | | CTCCCCATGGGGCAGCATCAGGACGGGGGCTCAAGCTGCACACGTGTGGA |
| | | CCCCTGCGGCAGGGTCGTGCTCCGTCTCCTGCGGGCGAGGTCTGATGGAG |
| | | CTCCGTTTCCTGTGCATGGACTCTGCCCTCAGGGTGCCTGTCCAGGAAGA |
| | | GCTGTGTGGCCTGGCAAGCAAGCCTGGGAGCCGGGGGGAGGTCTGCCAGG |
| | | CTGTCCCGTGCCCTGCTCGGTGGCAGTACAAGCTGGCGGCCTGCAGCGTG |
| | | AGCTGTGGGAGAGGGGTCGTGCGGAGGATCCTGTATTGTGCCCGGGCCCA |
| | | TGGGGAGGACGATGGTGAGGAGATCCTGTTGGACACCCAGTGCCAGGGGC |
| | | TGCCTCGCCCGGAACCCCAGGAGGCCTGCAGCCTGGAGCCCTGCCCACCT |
| | | AGGTGGAAAGTCATGTCCCTTGGCCCATGTTCGGCCAGCTGTGGCCTTGG |
| | | CACTGCTAGACGCTCGGTGGCCTGTGTGCAGCTCGACCAAGGCCAGGACG |
| | | TGGAGGTGGACGAGGCGGCCTGTGCGGCGCTGGTGCGGCCCGAGGCCAGT |
| | | GTCCCCTGTCTCATTGCCGACTGCACCTACCGCTGGCATGTTGGCACCTG |
| | | GATGGAGTGCTCTGTTTCCTGTGGGGATGGCATCCAGCGCCGGCGTGACA |
| | | CCTGCCTCGGACCCCAGGCCCAGGCGCCTGTGCCAGCTGATTTCTGCCAG |
| | | CACTTGCCCAAGCCGGTGACTGTGCGTGGCTGCTGGGCTGGGCCCTGTGT |
| | | GGGACAGGGTACGCCCAGCCTGGTGCCCCACGAAGAAGCCGCTGCTCCAG |
| | | GACGGACCACAGCCACCAAGGCTGGTGCCTCCCTGGAGTGGTCCCAGGCC |
| | | CGGGGCCTGCTCTTCTCCCCGGCTCCCCAGCCTCGGGGGCTCCTGCCCGG |
| | | GCCCCAGGAAAACTCAGTGCAGTCCAGTGCCTGTGGCAGGCAGCACCTTG |
| | | AGCCAACAGGAACCATTGACATGCGAGGCCCAGGGCAGGCAGACTGTGCA |
| | | GTGGCCATTGGGCGGCCCCTCGGGGAGGTGGTGACCCTCCGCGTCCTTGA |
| | | GAGTTCTCTCAACTGCAGTGCGGGGGACATGTTGCTGCTTTGGGGCCGGC |
| | | TCACCTGGAGGAAGATGTGCAGGAAGCTGTTGGACATGACTTTCAGCTCC |
| | | AAGACCAACACGCTGGTGGTGAGGCAGCGCTGCGGGCGGCCAGGAGGTGG |
| | | GGTGCTGCTGCGGTATGGGAGCCAGCTTGCTCCTGAAACCTTCTACAGAG |
| | | AATGTGACATGCAGCTCTTTGGGCCCTGGGGTGAAATCGTGAGCCCCTCG |
| | | CTGAGTCCAGCCACGAGTAATGCAGGGGGCTGCCGGCTCTTCATTAATGT |
| | | GGCTCCGCACGCACGGATTGCCATCCATGCCCTGGCCACCAACATGGGCG |
| | | CTGGGACCGAGGGAGCCAATGCCAGCTACATCTTGATCCGGGACACCCAC |
| | | AGCTTGAGGACCACAGCGTTCCATGGGCAGCAGGTGCTCTACTGGGAGTC |
| | | AGAGAGCAGCCAGGCTGAGATGGAGTTCAGCGAGGGCTTCCTGAAGGCTC |
| | | AGGCCAGCCTGCGGGGCCAGTACTGGACCCTCCAATCATGGGTACCGGAG |
| | | ATGCAGGACCCTCAGTCCTGGAAGGGAAAGGAAGGAACCTCTAGAGGGCC |
| | | CTTCGAACAAAAACTCATCTCAGAAGAGGATCTGAATATGCATACCGGTC |
| | | ATCATCACCATCACCAT |
| 125 | Exemplified P11541 | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGCGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCATTGCTGGTGCCTCCCTGGAGTGGTCCCAGGCCCGGGGCCT |
| | | GCTCTTCTCCCCGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGCGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 126 | Exemplified P1171K | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGCGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGGAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCAAGGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGGGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 127 | variant nucleotide | ATGCACCAGCGTCACCCCCGGGCAAGATGCCCTCCCCTCTGTGTGGCCGG |
| | sequence | AATCCTTGCCTGTGGCTTTCTCCTGGGCTGCTGGGGACCCTCCCATTTCC |
| | Exemplified P11711 | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGCCCTG |
| | | AGCCTGTGTGTGTCGGGCAGCTGCAGGACATTTGGCTGTGATGGTAGGAT |
| | | GGAGAGACAGCTTCCTCGATGGGACCCGGTGTATGCCAAGTGGCCCCCGG |
| | | GAGGACGGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCATTGCTCCCCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGGGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 128 | Exemplified P1173K | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGCTGCAGGCGGCATCCTACACCTGGAGCT |
| | | GCTGGTGGCCGTGGGCCCCGATGTCTTCCAGGCTCACCAGGAGGACACAG |
| | | AGCGCTATGTGCTCACCAACCTCAACATCGGGGCAGAACTGCTTCGGGAC |
| | | CCGTCCCTGGGGGCTCAGTTTCGGGTGCACCTGGTGAAGATGGTCATTCT |
| | | GACAGAGCCTGAGGGTGCTCCAAATATCACAGCCAACCTCACCTCGTCCC |
| | | TGCTGAGCGTCTGTGGGTGGAGCCAGACCATCAACCCTGAGGACGACACG |
| | | GATCCTGGCCATGCTGACCTGGTCCTCTATATCACTAGGTTTGACCTGGA |
| | | GTTGCCTGATGGTAACCGGCAGGTGCGGGGCGTCACCCAGCTGGGCGGTG |
| | | CCTGCTCCCCAACCTGGAGCTGCCTCATTACCGAGGACACTGGCTTCGAC |
| | | CTGGGAGTCACCATTGCCCATGAGATTGGGCACAGCTTCGGCCTGGAGCA |
| | | CGACGGCGCGCCCGGCAGCGGCTGCGGCCCCAGCGGACACGTGATGGCTT |
| | | CGGACGGCGCCGCGCCCCGCGCCGGCCTCGCCTGGTCCCCCTGCAGCCGC |
| | | CGGCAGCTGCTGAGCCTGCTCAGCGCAGGACGGGCGCGCTGCGTGTGGGA |
| | | CCCGCCGCGGCCTCAACCCGGGTCCGCGGGGCACCCGCCGGATGCGCAGC |
| | | CTGGCCTCTACTACAGCGCCAACGAGCAGTGCCGCGTGGCCTTCGGCCCC |
| | | AAGGCTGTCGCCTGCACCTTCGCCAGGGAGCACCTGGATATGTGCCAGGC |
| | | CCTCTCCTGCCACACAGACCCGCTGGACCAAAGCAGCTGCAGCCGCCTCC |
| | | TCGTTCCTCTCCTGGATGGGACAGAATGTGGCGTGGAGAAGTGGTGCTCC |
| | | AAAGGTCGCTGCCGCTCCCTGGTGGAGCTGACCCCCATAGCAGCAGTGCA |
| | | TGGGCGCTGGTCTAGCTGGGGTCCCCGAAGTCCTTGCTCCCGCTCCTGCG |
| | | GAGGAGGTGTGGTCACCAGGAGGCGGCAGTGCAACAACCCCAGACCTGCC |
| | | TTTGGGGGGCGTGCATGTGTTGGTGCTGACCTCCAGGCCGAGATGTGCAA |
| | | CACTCAGGCCTGCGAGAAGACCCAGCTGGAGTTCATGTCGCAACAGTGCG |
| | | CCAGGACCGACGGCCAGCCGCTGCGCTCCTCCCCTGGGGGCGCCTCCTTC |
| | | TACCACTGGGGTGCTGCTGTACCACACAGCCAAGGGGATGCTCTGTGCAG |
| | | ACACATGTGCCGGGCCATTGGCGAGAGCTTCATCATGAAGCGTGGAGACA |
| | | GCTTCCTCGATGGGACCCGGTGTATGCCAAGTGGCCCCCGGGAGGACGGG |
| | | ACTCCCAGCAGGTATGGGACAGGTGCCAGGTGTGTGGTGGGGACAACAGC |
| | | ACGTGCAGCCCTGAGCCTGTGTGTGTCGGGCAGCTGCAGGACATTTGGCT |
| | | GTGATGGTAGGATGGACCCACGGAAGGGCTCTTTCACAGCTGGCAGAGCG |
| | | AGAGAATATGTCACGTTTCTGACAGTTACCCCCAACCTGACCAGTGTCTA |
| | | CATTGCCAACCACAGGCCTCTCTTCACACACTTGGCGGTGAGGATCGGAG |
| | | GGCGCTATGTCGTGGCTGGGAAGATGAGCATCTCCCCTAACACCACCTAC |
| | | CCCTCCCTCCTGGAGGATGGTCGTGTCGAGTACAGAGTGGCCCTCACCGA |
| | | GGACCGGCTGCCCCGCCTGGAGGAGATCCGCATCTGGGGACCCCTCCAGG |
| | | AAGATGCTGACATCCAGGTTTACAGGCGGTATGGCGAGGAGTATGGCAAC |
| | | CTCACCCGCCCAGACATCACCTTCACCTACTTCCAGCCTAAGCCACGGCA |
| | | GGCCTGGGTGTGGGCCGCTGTGCGTGGGCCCTGCTCGGTGAGCTGTGGGG |
| | | CAGGGCTGCGCTGGGTAAACTACAGCTGCCTGGACCAGGCCAGGAAGGAG |
| | | TTGGTGGAGACTGTCCAGTGCCAAGGGAGCCAGCAGCCACCAGCGTGGCC |
| | | AGAGGCCTGCGTGCTCGAACCCTGCCCTCCCTACTGGGCGGTGGGAGACT |
| | | TCGGCCCATGCAGCGCCTCCTGTGGGGGTGGCCTGGGGGAGCGGCCAGTG |
| | | CGCTGCGTGGAGGCCCAGGGCAGCCTCCTGAAGACATTGCCCCCAGCCCG |
| | | GTGCAGAGCAGGGGCCCAGCAGCCAGGTGTGGCGCTGGAAACCTGCAACC |
| | | CCCAGCCCTGCCCTGCCAGGTGGGAGGTGTCAGAGCCCAGCTCATGCACA |
| | | TCAGCTGGTGGAGCAGGCCTGGCCTTGGAGAACGAGACCTGTGTGCCAGG |
| | | GGCAGATGGCCTGGAGGCTCCAGTGACTGAGGGGCCTGGCTCCGTAGATG |
| | | AGAAGCTGCCTGCCCCTGAGCCCTGTGTCGGGATGTCATGTCCTCCAGGC |
| | | TGGGGCCATCTGGATGCCACCTCTGCAGGGGAGAAGGCTCCCTCCCCATG |
| | | GGGCAGCATCAGGACGGGGGCTCAAGCTGCACACGTGTGGACCCCTGCGG |
| | | CAGGGTCGTGCTCCGTCTCCTGCGGGCGAGGTCTGATGGAGCTCCGTTTC |
| | | CTGTGCATGGACTCTGCCCTCAGGGTGCCTGTCCAGGAAGAGCTGTGTGG |
| | | CCTGGCAAGCAAGCCTGGGAGCCGGGGGGAGGTCTGCCAGGCTGTCCCGT |
| | | GCCCTGCTCGGTGGCAGTACAAGCTGGCGGCCTGCAGCGTGAGCTGTGGG |
| | | AGAGGGGTCGTGCGGAGGATCCTGTATTGTGCCCGGGCCCATGGGGAGGA |
| | | CGATGGTGAGGAGATCCTGTTGGACACCCAGTGCCAGGGGCTGCCTCGCC |
| | | CGGAACCCCAGGAGGCCTGCAGCCTGGAGCCCTGCCCACCTAGGTGGAAA |
| | | GTCATGTCCCTTGGCCCATGTTCGGCCAGCTGTGGCCTTGGCACTGCTAG |
| | | ACGCTCGGTGGCCTGTGTGCAGCTCGACCAAGGCCAGGACGTGGAGGTGG |
| | | ACGAGGCGGCCTGTGCGGGGCTGGTGCGGCCCGAGGCCAGTGTCCCCTGT |
| | | CTCATTGCCGACTGCACCTACCGCTGGCATGTTGGCACCTGGATGGAGTG |
| | | CTCTGTTTCCTGTGGGGATGGCATCCAGCGCCGGCGTGACACCTGCCTCG |
| | | GACCCCAGGCCCAGGCGCCTGTGCCAGCTGATTTCTGCCAGCACTTGCCC |
| | | AAGCCGGTGACTGTGCGTGGCTGCTGGGCTGGGCCCTGTGTGGGACAGGG |
| | | TACGCCCAGCCTGGTGCCCCACGAAGAAGCCGCTGCTCCAGGACGGACCA |
| | | CAGCCACCCCTGCTGGTGCCTCCCTGGAGTGGTCCCAGGCCCGGGGCCTG |
| | | CTCTTCTCCCCGGCTAAGCAGCCTCGGCGGCTCCTGCCCGGGCCCCAGGA |
| | | AAACTCAGTGCAGTCCAGTGCCTGTGGCAGGCAGCACCTTGAGCCAACAG |
| | | GAACCATTGACATGCGAGGCCCAGGGGAGGCAGACTGTGCAGTGGCCATT |
| | | GGGCGGCCCCTCGGGGAGGTGGTGACCCTCCGCGTCCTTGAGAGTTCTCT |
| | | CAACTGCAGTGCGGGGGACATGTTGCTGCTTTGGGGCCGGCTCACCTGGA |
| | | GGAAGATGTGCAGGAAGCTGTTGGACATGACTTTCAGCTCCAAGACCAAC |
| | | ACGCTGGTGGTGAGGCAGCGCTGCGGGGGGCCAGGAGGTGGGGTGCTGCT |
| | | GCGGTATGGGAGCCAGCTTGCTCCTGAAACCTTCTACAGAGAATGTGACA |
| | | TGCAGCTCTTTGGGCCCTGGGGTGAAATCGTGAGCCCCTCGCTGAGTCCA |
| | | GCCACGAGTAATGCAGGGGGCTGCCGGCTCTTCATTAATGTGGCTCCGCA |
| | | CGCACGGATTGCCATCCATGCCCTGGCCACCAACATGGGCGCTGGGACCG |
| | | AGGGAGCCAATGCCAGCTACATCTTGATCCGGGACACCCACAGCTTGAGG |
| | | ACCACAGCGTTCCATGGGCAGCAGGTGCTCTACTGGGAGTCAGAGAGCAG |
| | | CCAGGCTGAGATGGAGTTCAGCGAGGGCTTCCTGAAGGCTCAGGCCAGCC |
| | | TGCGGGGCCAGTACTGGACCCTCCAATCATGGGTACCGGAGATGCAGGAC |
| | | CCTCAGTCCTGGAAGGGAAAGGAAGGAACCTCTAGAGGGCCCTTCGAACA |
| | | AAAACTCATCTCAGAAGAGGATCTGAATATGCATACCGGTCATCATCACC |
| | | ATCACCAT |
| 129 | Exemplified P11731 | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGGTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTATTCAGCCTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGGGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 130 | Exemplified P1175K | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGGGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGGTGTGGCGCTGGAAACCTGCAAC |
| | | CCCCAGCCGTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGAAGCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 131 | Exemplified P11751 | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGCGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGCGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGATTCGGCGGCTCCTGCCCGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGGGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 132 | variant nucleotide | ATGCACCAGCGTCACCCCCGGGCAAGATGCCCTCCCCTCTGTGTGGCCGG |
| | Exemplified P1180K | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGCGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGTG |
| | | CCTGCTCCCCAACCTGGAGCTGCCTCATTACCGAGGACACTGGCTTCGAC |
| | | CTGGGAGTCACCATTGCCCATGAGATTGGGCACAGCTTCGGCCTGGAGCA |
| | | CGACGGCGCGCCCGGCAGCGGCTGCGGCCCCAGCGGACACGTGATGGCTT |
| | | CGGACGGCGCCGCGCCCCGCGCCGGCCTCGCCTGGTCCCCCTGCAGCCGC |
| | | CGGCAGCTGCTGAGCCTGCTCAGCGCAGGACGGGCGCGCTGCGTGTGGGA |
| | | CCCGCCGCGGCCTCAACCCGGGTCCGCGGGGCACCCGCCGGATGCGCAGC |
| | | CTGGCCTCTACTACAGCGCCAACGAGCAGTGCCGCGTGGCCTTCGGCCCC |
| | | AAGGCTGTCGCCTGCACCTTCGCCAGGGAGCACCTGGATATGTGCCAGGC |
| | | CCTCTCCTGCCACACAGACCCGCTGGACCAAAGCAGCTGCAGCCGCCTCC |
| | | TCGTTCCTCTCCTGGATGGGACAGAATGTGGCGTGGAGAAGTGGTGCTCC |
| | | AAAGGTCGCTGCCGCTCCCTGGTGGAGCTGACCCCCATAGCAGCAGTGCA |
| | | TGGGCGCTGGTCTAGCTGGGGTCCCCGAAGTCCTTGCTCCCGCTCCTGCG |
| | | GAGGAGGTGTGGTCACCAGGAGGCGGCAGTGCAACAACCCCAGACCTGCC |
| | | TTTGGGGGGCGTGCATGTGTTGGTGCTGACCTCCAGGCCGAGATGTGCAA |
| | | CACTCAGGCCTGCGAGAAGACCCAGCTGGAGTTCATGTCGCAACAGTGCG |
| | | CCAGGACCGACGGCCAGCCGCTGCGCTCCTCCCCTGGGGGCGCCTCCTTC |
| | | TACCACTGGGGTGCTGCTGTACCACACAGCCAAGGGGATGCTCTGTGCAG |
| | | ACACATGTGCCGGGCCATTGGCGAGAGCTTCATCATGAAGCGTGGAGACA |
| | | GCTTCCTCGATGGGACCCGGTGTATGCCAAGTGGCCCCCGGGAGGACGGG |
| | | ACCCTGAGCCTGTGTGTGTCGGGCAGCTGCAGGACATTTGGCTGTGATGG |
| | | TAGGATGGACTCCCAGCAGGTATGGGACAGGTGCCAGGTGTGTGGTGGGG |
| | | ACAACAGCACGTGCAGGGCGGTGAGGATCGGAGGGCGCTATGTCGTGGCT |
| | | GGGAAGATGAGCATCTCCCCTAACCCACGGAAGGGCTCTTTCACAGCTGG |
| | | CAGAGCGAGAGAATATGTCACGTTTCTGACAGTTACCCCCAACCTGACCA |
| | | GTGTCTACATTGCCAACCACAGGCCTCTCTTCACACACTTCACCACCTAC |
| | | CCCTCCCTCCTGGAGGATGGTCGTGTCGAGTACAGAGTGGCCCTCACCGA |
| | | GGACCGGCTGCCCCGCCTGGAGGAGATCCGCATCTGGGGACCCCTCCAGG |
| | | AAGATGCTGACATCCAGGTTTACAGGCGGTATGGCGAGGAGTATGGCAAC |
| | | CTCACCCGCCCAGACATCACCTTCACCTACTTCCAGCCTAAGCCACGGCA |
| | | GGCCTGGGTGTGGGCCGCTGTGCGTGGGCCCTGCTCGGTGAGCTGTGGGG |
| | | CAGGGCTGCGCTGGGTAAACTACAGCTGCCTGGACCAGGCCAGGAAGGAG |
| | | TTGGTGGAGACTGTCCAGTGCCAAGGGAGCCAGCAGCCACCAGCGTGGCC |
| | | AGAGGCCTGCGTGCTCGAACCCTGCCCTCCCTACTGGGCGGTGGGAGACT |
| | | TCGGCCCATGCAGCGCCTCCTGTGGGGGTGGCCTGCGGGAGCGGCCAGTG |
| | | CGCTGCGTGGAGGCCCAGGGCAGCCTCCTGAAGACATTGCCCCCAGCCCG |
| | | GTGCAGAGCAGGGGCCCAGCAGCCAGCTGTGGCGCTGGAAACCTGCAACC |
| | | CCCAGCCCTGCCCTGCCAGGTGGGAGGTGTCAGAGCCCAGCTCATGCACA |
| | | TCAGCTGGTGGAGCAGGCCTGGCCTTGGAGAACGAGACCTGTGTGCCAGG |
| | | GGCAGATGGCCTGGAGGCTCCAGTGACTGAGGGGCCTGGCTCCGTAGATG |
| | | AGAAGCTGCCTGCCCCTGAGCCCTGTGTCGGGATGTCATGTCCTCCAGGC |
| | | TGGGGCCATCTGGATGCCACCTCTGCAGGGGAGAAGGCTCCCTCCCCATG |
| | | GGGCAGCATCAGGACGGGGGCTCAAGCTGCACACGTGTGGACCCCTGCGG |
| | | CAGGGTCGTGCTCCGTCTCCTGCGGGCGAGGTCTGATGGAGCTCCGTTTC |
| | | CTGTGCATGGACTCTGCCCTCAGGGTGCCTGTCCAGGAAGAGCTGTGTGG |
| | | CCTGGCAAGCAAGCCTGGGAGCCGGGGGGAGGTCTGCCAGGCTGTCCCGT |
| | | GCCCTGCTCGGTGGCAGTACAAGCTGGCGGCCTGCAGCGTGAGCTGTGGG |
| | | AGAGGGGTCGTGCGGAGGATCCTGTATTGTGCCCGGGCCCATGGGGAGGA |
| | | CGATGGTGAGGAGATCCTGTTGGACACCCAGTGCCAGGGGCTGCCTCGCC |
| | | CGGAACCCCAGGAGGCCTGCAGCCTGGAGCCCTGCCCACCTAGGTGGAAA |
| | | GTCATGTCCCTTGGCCCATGTTCGGCCAGCTGTGGCCTTGGCACTGCTAG |
| | | ACGCTCGGTGGCCTGTGTGCAGCTCGACCAAGGCCAGGACGTGGAGGTGG |
| | | ACGAGGCGGCCTGTGCGGCGCTGGTGCGGCCCGAGGCCAGTGTCCCCTGT |
| | | CTCATTGCCGACTGCACCTACCGCTGGCATGTTGGCACCTGGATGGAGTG |
| | | CTCTGTTTCCTGTGGGGATGGCATCCAGCGCCGGCGTGACACCTGCCTCG |
| | | GACCCCAGGCCCAGGCGCCTGTGCCAGCTGATTTCTGCCAGCACTTGCCC |
| | | AAGCCGGTGACTGTGCGTGGCTGCTGGGCTGGGCCCTGTGTGGGACAGGG |
| | | TACGCCCAGCCTGGTGCCCCACGAAGAAGCCGCTGCTCCAGGACGGACCA |
| | | CAGCCACCCCTGCTGGTGCCTCCCTGGAGTGGTCCCAGGCCCGGGGCCTG |
| | | CTCTTCTCCCCGGCTCCCCAGCCTCGGCGGCTCCTGAAGGGGCCCCAGGA |
| | | AAACTCAGTGCAGTCCAGTGCCTGTGGCAGGCAGCACCTTGAGCCAACAG |
| | | GAACCATTGACATGCGAGGCCCAGGGGAGGCAGACTGTGCAGTGGCCATT |
| | | GGGGGGCCCCTCGGGGAGGTGGTGACCCTCCGCGTCCTTGAGAGTTCTCT |
| | | CAACTGCAGTGCGGGGGACATGTTGCTGCTTTGGGGCCGGCTCACCTGGA |
| | | GGAAGATGTGCAGGAAGCTGTTGGACATGACTTTCAGCTCCAAGACCAAC |
| | | ACGCTGGTGGTGAGGCAGCGCTGCGGGCGGCCAGGAGGTGGGGTGCTGCT |
| | | GCGGTATGGGAGCCAGCTTGCTCCTGAAACCTTCTACAGAGAATGTGACA |
| | | TGCAGCTCTTTGGGCCCTGGGGTGAAATCGTGAGCCCCTCGCTGAGTCCA |
| | | GCCACGAGTAATGCAGGGGGCTGCCGGCTCTTCATTAATGTGGCTCCGCA |
| | | CGCACGGATTGCCATCCATGCCCTGGCCACCAACATGGGCGCTGGGACCG |
| | | AGGGAGCCAATGCCAGCTACATCTTGATCCGGGACACCCACAGCTTGAGG |
| | | ACCACAGCGTTCCATGGGCAGCAGGTGCTCTACTGGGAGTCAGAGAGCAG |
| | | CCAGGCTGAGATGGAGTTCAGCGAGGGCTTCCTGAAGGCTCAGGCCAGCC |
| | | TGCGGGGCCAGTACTGGACCCTCCAATCATGGGTACCGGAGATGCAGGAC |
| | | CCTCAGTCCTGGAAGGGAAAGGAAGGAACCTCTAGAGGGCCCTTCGAACA |
| | | AAAACTCATCTCAGAAGAGGATCTGAATATGCATACCGGTCATCATCACC |
| | | ATCACCAT |
| 133 | Exemplified P11801 | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGG |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGATTGGGCCCCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGGGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGGGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 134 | Exemplified P1182K | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGGGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGCGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGGGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGCTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGGGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGAAGCAGG |
| | | AAAACTCAGTGCAGTCCAGTGCCTGTGGCAGGCAGCACCTTGAGCCAACA |
| | | GGAACCATTGACATGCGAGGCCCAGGGGAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGCGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 135 | Exemplified P11821 | ATGCACCAGCGTCACCCCCGGGCAAGATGCCCTCCCCTCTGTGTGGCCGG |
| | variant nucleotide | AATCCTTGCCTGTGGCTTTCTCCTGGGCTGCTGGGGACCCTCCCATTTCC |
| | sequence | AGCAGAGCTGTCTTCAGGCTTTGGAGCCACAGGCCGTGTCTTCTTACTTG |
| | | AGCCCTGGTGCTCCCTTAAAAGGCCGCCCTCCTTCCCCTGGCTTCCAGAG |
| | | GCAGAGGCAGAGGCAGAGGGGGGCTGCAGGCGGCATCCTACACCTGGAGC |
| | | TGCTGGTGGCCGTGGGCCCCGATGTCTTCCAGGCTCACCAGGAGGACACA |
| | | GAGCGCTATGTGCTCACCAACCTCAACATCGGGGCAGAACTGCTTCGGGA |
| | | CCCGTCCCTGGGGGCTCAGTTTCGGGTGCACCTGGTGAAGATGGTCATTC |
| | | TGACAGAGCCTGAGGGTGCTCCAAATATCACAGCCAACCTCACCTCGTCC |
| | | CTGCTGAGCGTCTGTGGGTGGAGCCAGACCATCAACCCTGAGGACGACAC |
| | | GGATCCTGGCCATGCTGACCTGGTCCTCTATATCACTAGGTTTGACCTGG |
| | | AGTTGCCTGATGGTAACCGGCAGGTGCGGGGCGTCACCCAGCTGGGCGGT |
| | | GCCTGCTCCCCAACCTGGAGCTGCCTCATTACCGAGGACACTGGCTTCGA |
| | | CCTGGGAGTCACCATTGCCCATGAGATTGGGCACAGCTTCGGCCTGGAGC |
| | | ACGACGGCGCGCCCGGCAGCGGCTGCGGCCCCAGGGGACACGTGATGGCT |
| | | TCGGACGGCGCCGCGCCCCGCGCCGGCCTCGCCTGGTCCCCCTGCAGCCG |
| | | CCGGCAGCTGCTGAGCCTGCTCAGCGCAGGACGGGCGCGCTGCGTGTGGG |
| | | ACCCGCCGCGGCCTCAACCCGGGTCCGCGGGGCACCCGCCGGATGCGCAG |
| | | CCTGGCCTCTACTACAGCGCCAACGAGCAGTGCCGCGTGGCCTTCGGCCC |
| | | CAAGGCTGTCGCCTGCACCTTCGCCAGGGAGCACCTGGATATGTGCCAGG |
| | | CCCTCTCCTGCCACACAGACCCGCTGGACCAAAGCAGCTGCAGCCGCCTC |
| | | CTCGTTCCTCTCCTGGATGGGACAGAATGTGGCGTGGAGAAGTGGTGCTC |
| | | CAAAGGTCGCTGCCGCTCCCTGGTGGAGCTGACCCCCATAGCAGCAGTGC |
| | | ATGGGCGCTGGTCTAGCTGGGGTCCCCGAAGTCCTTGCTCCCGCTCCTGC |
| | | GGAGGAGGTGTGGTCACCAGGAGGCGGCAGTGCAACAACCCCAGACCTGC |
| | | CTTTGGGGGGCGTGCATGTGTTGGTGCTGACCTCCAGGCCGAGATGTGCA |
| | | ACACTCAGGCCTGCGAGAAGACCCAGCTGGAGTTCATGTCGCAACAGTGC |
| | | GCCAGGACCGACGGCCAGCCGCTGCGCTCCTCCCCTGGCGGCGCCTCCTT |
| | | CTACCACTGGGGTGCTGCTGTACCACACAGCCAAGGGGATGCTCTGTGCA |
| | | GACACATGTGCCGGGCCATTGGCGAGAGCTTCATCATGAAGCGTGGAGAC |
| | | AGCTTCCTCGATGGGACCCGGTGTATGCCAAGTGGCCCCCGGGAGGACGG |
| | | GACCCTGAGCCTGTGTGTGTCGGGCAGCTGCAGGACATTTGGCTGTGATG |
| | | GTAGGATGGACTCCCAGCAGGTATGGGACAGGTGCCAGGTGTGTGGTGGG |
| | | GACAACAGCACGTGCAGCCCACGGAAGGGCTCTTTCACAGCTGGCAGAGC |
| | | GAGAGAATATGTCACGTTTCTGACAGTTACCCCCAACCTGACCAGTGTCT |
| | | ACATTGCCAACCACAGGCCTCTCTTCACACACTTGGCGGTGAGGATCGGA |
| | | GGGCGCTATGTCGTGGCTGGGAAGATGAGCATCTCCCCTAACACCACCTA |
| | | CCCCTCCCTCCTGGAGGATGGTCGTGTCGAGTACAGAGTGGCCCTCACCG |
| | | AGGACCGGCTGCCCCGCCTGGAGGAGATCCGCATCTGGGGACCCCTCCAG |
| | | GAAGATGCTGACATCCAGGTTTACAGGCGGTATGGCGAGGAGTATGGCAA |
| | | CCTCACCCGCCCAGACATCACCTTCACCTACTTCCAGCCTAAGCCACGGC |
| | | AGGCCTGGGTGTGGGCCGCTGTGCGTGGGCCCTGCTCGGTGAGCTGTGGG |
| | | GCAGGGCTGCGCTGGGTAAACTACAGCTGCCTGGACCAGGCCAGGAAGGA |
| | | GTTGGTGGAGACTGTCCAGTGCCAAGGGAGCCAGCAGCCACCAGCGTGGC |
| | | CAGAGGCCTGCGTGCTCGAACCCTGCCCTCCCTACTGGGCGGTGGGAGAC |
| | | TTCGGCCCATGCAGCGCCTCCTGTGGGGGTGGCCTGGGGGAGCGGCCAGT |
| | | GCGCTGCGTGGAGGCCCAGGGCAGCCTCCTGAAGACATTGCCCCCAGCCC |
| | | GGTGCAGAGCAGGGGCCCAGCAGCCAGGTGTGGCGCTGGAAACCTGCAAC |
| | | CCCCAGCCCTGCCCTGCCAGGTGGGAGGTGTCAGAGCCCAGCTCATGCAC |
| | | ATCAGCTGGTGGAGCAGGCCTGGCCTTGGAGAACGAGACCTGTGTGCCAG |
| | | GGGCAGATGGCCTGGAGGCTCCAGTGACTGAGGGGCCTGGCTCCGTAGAT |
| | | GAGAAGCTGCCTGCCCCTGAGCCCTGTGTCGGGATGTCATGTCCTCCAGG |
| | | CTGGGGCCATCTGGATGCCACCTCTGCAGGGGAGAAGGCTCCCTCCCCAT |
| | | GGGGCAGCATCAGGACGGGGGCTCAAGCTGCACACGTGTGGACCCCTGCG |
| | | GCAGGGTCGTGCTCCGTCTCCTGCGGGCGAGGTCTGATGGAGCTCCGTTT |
| | | CCTGTGCATGGACTCTGCCCTCAGGGTGCCTGTCCAGGAAGAGCTGTGTG |
| | | GCCTGGCAAGCAAGCCTGGGAGCCGGCGGGAGGTCTGCCAGGCTGTCCCG |
| | | TGCCCTGCTCGGTGGCAGTACAAGCTGGCGGCCTGCAGCGTGAGCTGTGG |
| | | GAGAGGGGTCGTGCGGAGGATCCTGTATTGTGCCCGGGCCCATGGGGAGG |
| | | ACGATGGTGAGGAGATCCTGTTGGACACCCAGTGCCAGGGGCTGCCTCGC |
| | | CCGGAACCCCAGGAGGCCTGCAGCCTGGAGCCCTGCCCACCTAGGTGGAA |
| | | AGTCATGTCCCTTGGCCCATGTTCGGCCAGCTGTGGCCTTGGCACTGCTA |
| | | GACGCTCGGTGGCCTGTGTGCAGCTCGACCAAGGCCAGGACGTGGAGGTG |
| | | GACGAGGCGGCCTGTGCGGCGCTGGTGCGGCCCGAGGCCAGTGTCCCCTG |
| | | TCTCATTGCCGACTGCACCTACCGCTGGCATGTTGGCACCTGGATGGAGT |
| | | GCTCTGTTTCCTGTGGGGATGGCATCCAGCGCCGGCGTGACACCTGCCTC |
| | | GGACCCCAGGCCCAGGCGCCTGTGCCAGCTGATTTCTGCCAGCACTTGCC |
| | | CAAGCCGGTGACTGTGCGTGGCTGCTGGGCTGGGCCCTGTGTGGGACAGG |
| | | GTACGCCCAGCCTGGTGCCCCACGAAGAAGCCGCTGCTCCAGGACGGACC |
| | | ACAGCCACCCCTGCTGGTGCCTCCCTGGAGTGGTCCCAGGCCCGGGGCCT |
| | | GCTCTTCTCCCCGGCTCCCCAGCCTCGGCGGCTCCTGCCCGGGATTCAGG |
| | | AAAACTCAGTGCAGTCCAGTGCCTGTGGCAGGCAGCACCTTGAGCCAACA |
| | | GGAACCATTGACATGCGAGGCCCAGGGCAGGCAGACTGTGCAGTGGCCAT |
| | | TGGGCGGCCCCTCGGGGAGGTGGTGACCCTCCGCGTCCTTGAGAGTTCTC |
| | | TCAACTGCAGTGCGGGGGACATGTTGCTGCTTTGGGGCCGGCTCACCTGG |
| | | AGGAAGATGTGCAGGAAGCTGTTGGACATGACTTTCAGCTCCAAGACCAA |
| | | CACGCTGGTGGTGAGGCAGCGCTGGGGGGGGCCAGGAGGTGGGGTGCTGC |
| | | TGCGGTATGGGAGCCAGCTTGCTCCTGAAACCTTCTACAGAGAATGTGAC |
| | | ATGCAGCTCTTTGGGCCCTGGGGTGAAATCGTGAGCCCCTCGCTGAGTCC |
| | | AGCCACGAGTAATGCAGGGGGCTGCCGGCTCTTCATTAATGTGGCTCCGC |
| | | ACGCACGGATTGCCATCCATGCCCTGGCCACCAACATGGGCGCTGGGACC |
| | | GAGGGAGCCAATGCCAGCTACATCTTGATCCGGGACACCCACAGCTTGAG |
| | | GACCACAGCGTTCCATGGGCAGCAGGTGCTCTACTGGGAGTCAGAGAGCA |
| | | GCCAGGCTGAGATGGAGTTCAGCGAGGGCTTCCTGAAGGCTCAGGCCAGC |
| | | CTGCGGGGCCAGTACTGGACCCTCCAATCATGGGTACCGGAGATGCAGGA |
| | | CCCTCAGTCCTGGAAGGGAAAGGAAGGAACCTCTAGAGGGCCCTTCGAAC |
| | | AAAAACTCATCTCAGAAGAGGATCTGAATATGCATACCGGTCATCATCAC |
| | | CATCACCAT |
| 136 | Human ADAMTS13 | KAAPGRTTATPAGASLEWSQARGLLFSPAPQPRRLLPGP |
| | A1144K L3 | |
| | subregion | |
| 137 | Human ADAMTS13 | AKAPGRTTATPAGASLEWSQARGLLFSPAPQPRRLLPGP |
| | A1145K L3 | |
| | subregion | |
| 138 | Human ADAMTS13 | AAKPGRTTATPAGASLEWSQARGLLFSPAPQPRRLLPGP |
| | A1146K L3 | |
| | subregion | |
| 139 | Human ADAMTS13 | AAAKGRTTATPAGASLEWSQARGLLFSPAPQPRRLLPGP |
| | P1147K L3 | |
| | subregion | |
| 140 | Human ADAMTS13 | AAAPGRTTATKAGASLEWSQARGLLFSPAPQPRRLLPGP |
| | P1154K L3 | |
| | subregion | |
| 141 | Human ADAMTS13 | AAAPGRTTATPAGASLEWSQARGLLFSKAPQPRRLLPGP |
| | P1171K L3 | |
| | subregion | |
| 142 | Human ADAMTS13 | AAAPGRTTATPAGASLEWSQARGLLFSPAKQPRRLLPGP |
| | P1173K L3 | |
| | subregion | |
| 143 | Human ADAMTS13 | AAAPGRTTATPAGASLEWSQARGLLFSPAPQKRRLLPGP |
| | P1175K L3 | |
| | subregion | |
| 144 | Human ADAMTS13 | AAAPGRTTATPAGASLEWSQARGLLFSPAPQPRRLLKGP |
| | P1180K L3 | |
| | subregion | |
| 145 | Human ADAMTS13 | AAAPGRTTATPAGASLEWSQARGLLFSPAPQPRRLLPGK |
| | P1182K L3 | |
| | subregion | |
| 146 | Human ADAMTS13 | IAAPGRTTATPAGASLEWSQARGLLFSPAPQPRRLLPGP |
| | A11441 L3 subregion | |
| 147 | Human ADAMTS13 | AIAPGRTTATPAGASLEWSQARGLLFSPAPQPRRLLPGP |
| | A11451 L3 subregion | |
| 148 | Human ADAMTS13 | AAIPGRTTATPAGASLEWSQARGLLFSPAPQPRRLLPGP |
| | A11461 L3 subregion | |
| 149 | Human ADAMTS13 | AAAIGRTTATPAGASLEWSQARGLLFSPAPQPRRLLPGP |
| | P11471 L3 subregion | |
| 150 | Human ADAMTS13 | AAAPGRTTATIAGASLEWSQARGLLFSPAPQPRRLLPGP |
| | P11541 L3 subregion | |
| 151 | Human ADAMTS13 | AAAPGRTTATPAGASLEWSQARGLLFSIAPQPRRLLPGP |
| | P11711 L3 subregion | |
| 152 | Human ADAMTS13 | AAAPGRTTATPAGASLEWSQARGLLFSPAIQPRRLLPGP |
| | P11731 L3 subregion | |
| 153 | Human ADAMTS13 | AAAPGRTTATPAGASLEWSQARGLLFSPAPQIRRLLPGP |
| | P11751 L3 subregion | |
| 154 | Human ADAMTS13 | AAAPGRTTATPAGASLEWSQARGLLFSPAPQPRRLLIGP |
| | P11801 L3 subregion | |
| 155 | Human ADAMTS13 | AAAPGRTTATPAGASLEWSQARGLLFSPAPQPRRLLPGI |
| | P11821 L3 subregion | |
| 156 | Human ADAMTS13 | X₁X₂X₃X₄GRTTATX₅AGASLEWSQARGLLFSX₆AX₇QX₈RRLLX₉GX₁₀ |
| | variant L3 | wherein X₁ = A, K, I, or V, X₂ = A, K, I, or V, |
| | subregion | X₃ = A, K, I, or V, X₄ = P, K, I, or V, |
| | consensus 2 | X₅ = P, K, I, or V, |
| | | X₆ = P, K, I, or V, X₇ = P, K, I, or V, |
| | | X₈ = P, K, I, or V, X₉ = P, K, I, or V and |
| | | X₁₀ = P, K, I, |
| | | or V |

### SUMMARY OF THE FIGURES

Embodiments and experiments illustrating the principles of the present disclosure will now be discussed with reference to the accompanying figures in which:

FIG. 1A. In vitro activities of ADAMTS13 linker 3 variants. The proteolytic activities of wild type (wt) ADAMTS13, the gain of function (GoF) ADAMTS13 variant and linker 3 variants were determined by FRETS-VWF73 assay in both the absence (−) and presence (+) of the activating VWF-D4CK domain fragment. All activities are presented relative to the basal activity of wtADAMTS13 (100%).

FIG. 1B. In vitro activities of further ADAMTS13 linker 3 variants. The proteolytic activities of wild type (wt) ADAMTS13 and the indicated linker 3 variants were determined by FRETS-VWF73 assay in both the absence (hollow bars) and presence (textured bars) of the activating VWF-D4CK domain fragment. All activities are presented relative to the basal activity of wtADAMTS13 (100%), as denoted by a dotted line.

FIG. 2. The VWF-mediated capture of platelets under arterial shear stress was determined in the presence of either wtADAMTS13, GoF ADAMTS13 or the linker 3 variant A1144V ADAMTS13 at a range of concentrations. For comparison, the results of VWF negative controls are also shown.

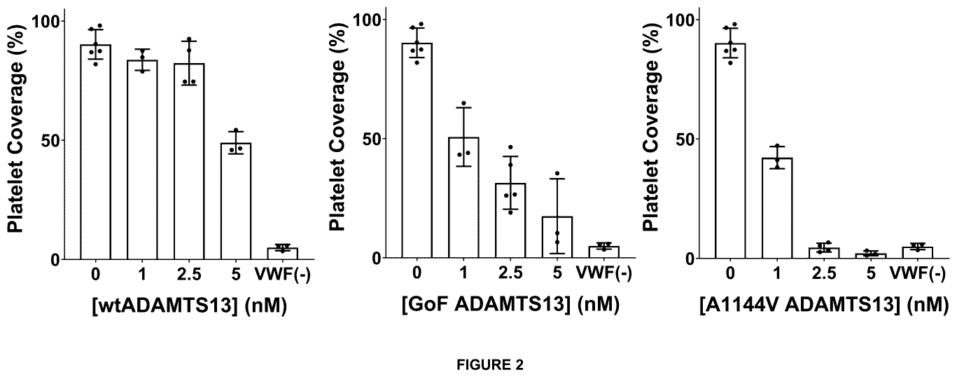

FIG. 3. Restoration of rCBF to the MCA territory by the A1144V ADAMTS13 variant (caADAMTS13) administered 1 hour after MCA occlusion. A, raw laser speckle contrast images acquired at 0, 30 and 60 minutes post-injection of either vehicle control, N-acetyl-cysteine (NAC), wtADAMTS13 or caADAMTS13. Regions of interest (ROI), in the MCA territories of the contralateral (contra) and ipsilateral (ipsi) hemispheres, were used for rCBF quantification.

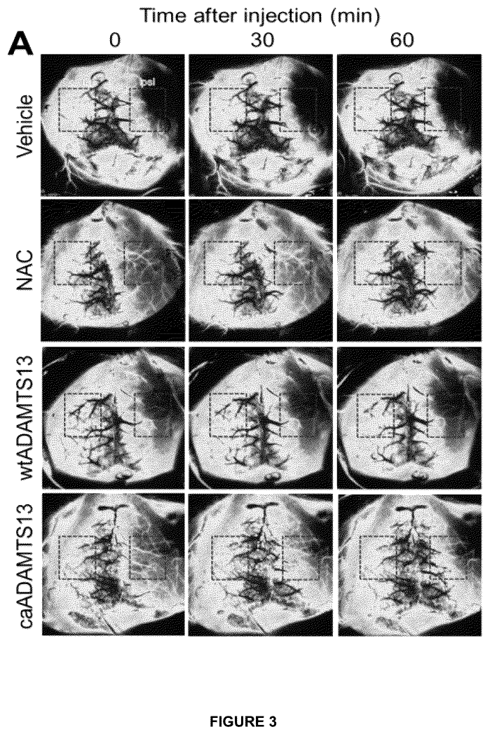

FIG. 4. Flux values for contralateral and ipsilateral ROIs were determined at 1 minute intervals for 5 minutes prior to injection and for 60 minutes post-injection. Traces shown are representative measurements from a single animal in each treatment group.

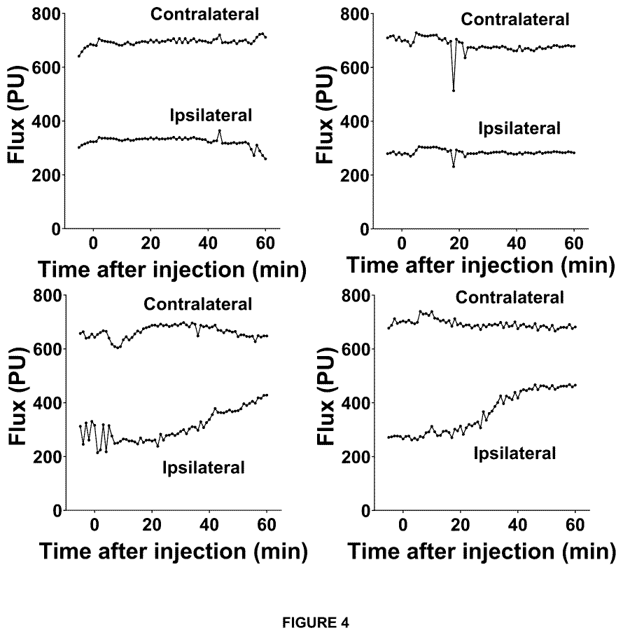

FIG. 5. The change in average flux ratio (ipsi/contra) over the course of the experiment was determined for each treatment group. An arbitrary flux ratio value of 0.5 was used to confirm successful MCA occlusion and as the point at which recanalisation was achieved in treated animals (*). For comparison the flux ratio of a sham (i.e. non-stroked) animal is also shown.

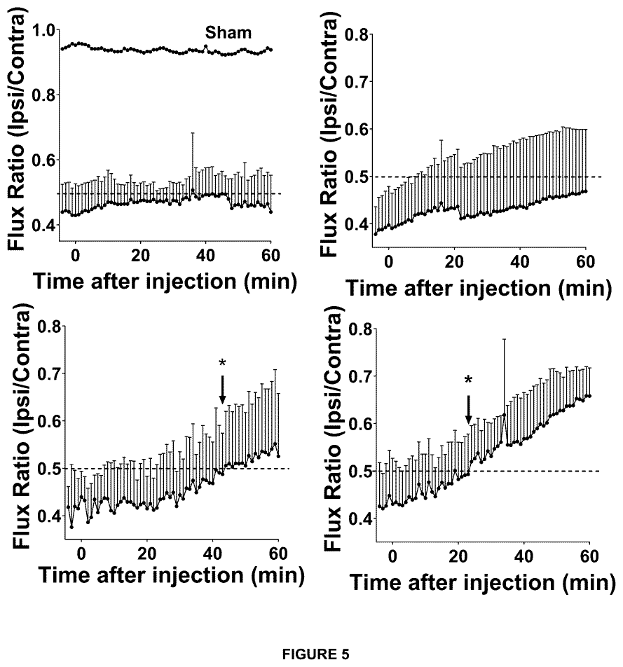

FIG. 6. Impact of treatments on lesion volumes determined 24 hours post-occlusion. Lesion volumes were measured using cresyl violet stained brain sections. For comparison, sections from a sham animal, in which FeCl₃ was not applied to the MCA, are also shown.

FIG. 7. Lesion volumes in each treatment group were compared to the vehicle treated group using an unpaired t-test with Welch's correction (* p<0.01). In this case sham animals are those excluded from the study due to lack of stable MCA occlusion. The % increase, between 0 and 60 minutes after injection, in flux ratio between the ipsilateral and contralateral ROIs was also compared between vehicle and each treatment group (* p<0.01).

FIG. 8. A significant negative correlation was observed between the increase in flux ratio and lesion volume across all groups. Animals treated with caADAMTS13 are highlighted in red.

FIG. 9. Haematoxylin and eosin stained brain sections were used to identify evidence of haemorrhagic transformation 24 hours post-treatment.

FIG. 10. Impact of treatments on residual thrombus content at site of FeCl₃ application 24 hours post-occlusion. Whole brain sections were stained by immunofluorescence to visualise VWF and fibrin(ogen). Higher magnification of marked ROIs. Areas of microvascular VWF-platelet deposition are shown by white arrows.

FIG. 11. The total deposition of VWF and fibrin in the entire region adjacent to the MCA was quantified and compared between vehicle and treatment groups using an unpaired t-test with Welch's correction (*<0.05, **p<0.01). Sham animals are those in which FeCl₃ was not applied to the MCA.

FIG. 12. Male CD1 mice, aged 13-14 weeks, were infected with Streptococcus pneumoniae using an ascending infectious challenge over a 6 day period. On day 8 they were treated with either vehicle (no treatment) or A1144V ADAMTS13 (caADAMTS13) by tail vein injection. This was performed in mice with ongoing infection (A) and in mice in which the infection was resolved by a single sub-cutaneous injection of amoxicillin (B). On day 18 3D MGE data was acquired two hours following intravenous administration of α-Von Willebrand Factor (VWF)-conjugated MPIO particles. MGE data was fit using a mono-exponential model to estimate R2* maps. Whole brain masks were applied and values of total R2* were derived from the entire brain volume (C). The reactivity of VWF (VWF:CBA) in the plasma of these experimental animals (relative to that of mock infected control animals) was also determined (D). Untreated and amoxicillin treated mice show higher levels of binding of α-VWF MPIO particles (higher R2*) compared to mock infected animals, indicating increased endothelial activation in the cerebral vasculature. This is accompanied by increased levels of reactive VWF species in the plasma. Administration of caADAMTS13 significantly reduces levels of VWF detected in the cerebral vasculature and plasma in animals with resolved or ongoing infection. Treatment groups were compared as indicated using an unpaired t-test with Welch's correction (*<0.05, **p<0.01, ****p<0.001).

### DETAILED DESCRIPTION OF THE DISCLOSURE

Aspects and embodiments of the present disclosure will now be discussed with reference to the accompanying figures. Further aspects and embodiments will be apparent to those skilled in the art. All documents mentioned in this text are incorporated herein by reference.

The present disclosure is based on the identification by the inventors that the linker 3 region of ADAMTS13 can be modified to improve the therapeutic properties of the protein. The inventors have generated multiple ADAMTS13 variants with amino acid substitutions in the linker 3 region that have improved characteristics compared to wildtype ADAMTS13.

The variants of the present disclosure have a number of advantages over wildtype ADAMTS13, recombinant wildtype ADAMTS13, and the known GoF ADAMTS13 variant, including: the lack of requirement for substrate-induced activation, broadened substrate specificity, and higher enzymatic activity. The variants of the present disclosure have higher proteolytic activity compared to wildtype ADAMTS13 and the known GoF ADAMTS13 variant. Variants have been shown to have efficacy at lower doses compared to wildtype ADAMTS13 and the known GoF ADAMTS13 variant. Additionally, unlike the known GoF ADAMTS13 variant, the variants of the present disclosure have a fully functional spacer exosite and remain in the optimally activated state.

The only approved treatment for Acute Ischaemic Stroke (AIS) is recombinant tissue plasminogen activator (rt-PA), however the use of rt-PA increases the risk of intracerebral haemorrhage in up to 7% of AIS patients (Yaghi et al. 2017). The risk of haemorrhage rises proportionally with stroke severity and delay in administration (Whiteley et al. 2016). Crucially, the inventors and others have found no evidence of haemorrhagic transformation after treatment with either wildtype ADAMTS13 or ADAMTS13 variants in murine models of AIS (Denorme et al. 2016; Nakano et al. 2015). In a murine model of haemorrhagic stroke, ADAMTS13 administration has no effect on bleeding and may, in fact, be of therapeutic potential in this condition (Zhao et al. 2009).

### General Definitions

As used herein, a “fragment”, “variant” or “homologue” of a given protein may optionally be characterised as having at least 40%, preferably one of 45%, 50%, 55%, 60%, 70%, 75%, 80%, 85%, 90%, 91%, 92%, 93%, 94%, 95%, 96%, 97%, 98%, 99% or 100% amino acid sequence identity to the amino acid sequence of the reference protein. Fragments, variants, isoforms and homologues of a reference protein may be characterised by the ability to perform a function performed by the reference protein.

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purpose of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

A “variant” generally refers to a protein having an amino acid sequence comprising one or more amino acid substitutions, insertions, deletions or other modifications relative to the amino acid sequence of the reference protein, but retaining a considerable degree of sequence identity (e.g. at least 40%) to the amino acid sequence of the reference protein. An “isoform” generally refers to a variant of the reference protein expressed by the same species as the species of the reference protein. A “homologue” generally refers to a variant of the reference protein produced by a different species as compared to the species of the reference protein.

A “fragment” of a reference protein may be of any length (by number of amino acids), although may optionally be at least 25% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein.

Regions and positions of a given polypeptide/amino acid sequence which “corresponding to” those of a reference amino acid sequence can be identified by sequence alignment, which can be performed e.g. using sequence alignment software such as ClustalOmega (Söding, J. 2005, Bioinformatics 21, 951-960). A region of a given polypeptide/amino acid sequence which corresponds to a region of a reference an amino acid sequence may have at least 60%, preferably one of 70%, 75%, 80%, 85%, 90%, 91%, 92%, 93%, 94%, 95%, 96%, 97%, 98%, 99% or 100% amino acid sequence identity to the region of the reference an amino acid sequence to which it corresponds. By way of illustration, the amino acid sequence shown in SEQ ID NO:48 corresponds to positions 1131 to 1190 of SEQ ID NO:1. By way of further illustration, the alanine residue at position 14 of SEQ ID NO:48 corresponds to positions 1144 of SEQ ID NO:1.

### ADAMTS13 and Von Willebrand Factor (VWF)

ADAMTS13 (a disintegrin-like and metalloprotease with thrombospondin type 1 motif 13) is identified by UniProtKB Q76LX8. Alternative splicing of mRNA encoded by the human ADAMTS13 gene yields four isoforms: isoform 1 (SEQ ID NO:1); isoform 2 (SEQ ID NO:2) lacking positions 1135-1190 relative to isoform 1; isoform 3 (SEQ ID NO:3) lacking positions 275-305 and 1135-1190 relative to isoform 1; and isoform 4 (SEQ ID NO:4) lacking positions 2-329 and 693-1427 relative to isoform 1, and having a variant sequence at the positions relative to positions 258-692 of isoform 1.

The structure and function of ADAMTS13 Is reviewed e.g. In Chen et al., Front Neurol. (2019) 10: 772, which is hereby incorporated by reference in its entirety. ADAMTS13 is a 1,427 amino acid metalloprotease comprising: a signal peptide (SEQ ID NO:5), a short propeptide domain (SEQ ID NO:6), a metalloprotease domain (SEQ ID NO:13), a disintegrin-like domain (SEQ ID NO:14), a thrombospondin-type 1 (TSP1) repeat domain (SEQ ID NO:15), a cysteine-rich domain (SEQ ID NO:16), a spacer domain (SEQ ID NO:17), seven further TSP1 repeat domains (SEQ ID NOs:18 to 24), and two CUB domains (SEQ ID NOs:25 and 26).

ADAMTS13 Is the cleaving protease of von Willebrand factor (VWF), a key factor in thrombus formation. The biological breakdown (catabolism) of VWF (e.g. within endovascular platelet aggregates) is largely mediated by the enzyme ADAMTS13.

VWF is identified by UniProtKB P04275. Alternative splicing of mRNA encoded by the human VWF gene yields two isoforms: isoform 1 (SEQ ID NO:27); Isoform 2 (SEQ ID NO:28) having variant sequences at the positions corresponding to positions 1-18 and 220-314 of isoform 1, and lacking positions 315-2813 relative to isoform 1.

The structure and function of VWF is described e.g. in Bharati and Prashanth, Indian J Pharm Sci. (2011) 73(1): 7-16 and Zhou et al., Blood. (2012) 120(2): 449-458, both of which are hereby incorporated by reference in their entirety. The protein encoded by VWF comprises a signal peptide (SEQ ID NO-29), von Willebrand antigen 2 (SEQ ID NO:31; a propeptide) and the mature von Willebrand factor (SEQ ID NO:32).

Von Willebrand antigen 2 comprises VWFD1, TIL1, VWFD2 and TIL2 domains (SEQ ID NOs:33 to 36). Mature VWF comprises TIL3, VWFD3, TIL4, VWFA1, VWFA2, VWFA3, VWFD4, VWFC1, VWFC2, VWFC3 and CTCK domains (SEQ ID NOs:37 to 47).

VWF is typically expressed as a large multimeric glycoprotein complexes which are released by endothelial cells in the form of ultra-large multimers (UL-VWF) of varying sizes, having molecular weights of up to 20,000 kDa.

The metalloprotease domain of ADAMTS13 is responsible for recognition and cleavage of VWF, at the peptide bond formed between positions Y1605 and M1606 (numbering relative to SEQ ID N027), within the VWFA2 domain of VWF. The region of ADAMTS13 comprising the disintegrin-like domain, TSP1 repeat 1, the cysteine-rich domain and the spacer domain is implicated in the binding of ADAMTS13 to the VWFA2 domain of VWF. The region of ADAMTS13 comprising TSP1 repeats 5 to 8 and CUB domains 1 and 2 is implicated in the binding of ADAMTS13 to the region of VWF comprising VWFD4, VWFC1, VWFC2, VWFC3 and CTCK domains.

ADAMTS13-mediated cleavage of VWF is crucial for blood homeostasis. VWF plays a major role in blood coagulation. It binds to other proteins, in particular factor VIII, and it is implicated in platelet adhesion at wound sites. High levels of VWF can lead to diseases such as stroke as well as other thrombotic disorders.

Dysfunction of ADAMTS13-mediated cleavage of VWF leads to VWF accumulation and adhesion of platelets, resulting in thrombosis and inflammation. In pathologic states such as thrombotic thrombocytopenic purpura (TTP) and other thrombotic microangiopathies (TMAs), VWF binds to the endothelium and forms large multimers. As the anchored VWF chains grow, they provide a greater surface area to bind circulating platelets (PLTs), generating unique thrombi characteristic of TTP. This results in microvasculature thrombosis, obstruction of blood flow, and ultimately tissue and organ damage. TTP and TMAs can arise e.g. as a consequence of autoimmune responses towards ADAMTS13, or as a result of ADAMTS13 deficiency.

In this specification “ADAMTS13” refers to ADAMTS13 from any species and includes ADAMTS13 isoforms, fragments, variants or homologues from any species.

In some embodiments, the ADAMTS13 is ADAMTS13 from a mammal (e.g. a primate (rhesus, cynomolgous, or human) and/or a rodent (e.g. rat or murine) ADAMTS13). Isoforms, fragments, variants or homologues of ADAMTS13 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature ADAMTS13 isoform from a given species, e.g. human. In preferred embodiments the ADAMTS13 is a human ADAMTS13 isoform (e.g. isoform 1, 2, 3 or 4).

Isoforms, fragments, variants or homologues may optionally be functional isoforms, fragments, variants or homologues, e.g. having a functional property/activity of the reference ADAMTS13 (e.g. human ADAMTS13 isoform 1), as determined by analysis by a suitable assay for the functional property/activity. For example, an isoform, fragment, variant or homologue of ADAMTS13 may display association with and/or cleavage of human VWF.

In some embodiments, the ADAMTS13 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:1, 2, 3, 4, 7, 8, 9, 10, 11 or 12.

In this specification “VWF” refers to VWF from any species and includes VWF isoforms, fragments, variants or homologues from any species.

In some embodiments, the VWF is VWF from a mammal (e.g. a primate (rhesus, cynomolgous, or human) and/or a rodent (e.g. rat or murine) VWF). Isoforms, fragments, variants or homologues of VWF may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature VWF isoform from a given species, e.g. human. In preferred embodiments the VWF is a human VWF isoform (e.g. isoform 1 or 2).

Isoforms, fragments, variants or homologues may optionally be functional isoforms, fragments, variants or homologues, e.g. having a functional property/activity of the reference VWF (e.g. human VWF isoform 1), as determined by analysis by a suitable assay for the functional property/activity. For example, an isoform, fragment, variant or homologue of VWF may display association with and/or may be susceptible to cleavage by human ADAMTS13.

In some embodiments, the VWF comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to SEQ ID NO:27, 28, 30 or 32.

### ADAMTS13 Variants

Aspects and embodiments of the present disclosure relate to variants of ADAMTS13.

As used herein, “an ADAMTS13 variant” refers to a polypeptide comprising, or consisting of, an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to the amino acid sequence of wildtype human ADAMTS13, and comprising one or more amino acid substitutions relative to the amino acid sequence of wildtype human ADAMTS13.

In some embodiments, an ADAMTS13 variant according to the present disclosure is a polypeptide comprising, or consisting of, an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:1, 2, 3, 4, 7, 8, 9, 10, 11 or 12, and comprising one or more amino acid substitutions relative to the reference sequence.

In preferred embodiments, an ADAMTS13 variant according to the present disclosure is a polypeptide comprising, or consisting of, an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:1, 7 or 8, and comprising one or more amino acid substitutions relative to the reference sequence.

In some embodiments, an ADAMTS13 variant comprises, or consists of, an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:1, and comprising one or more amino acid substitutions relative to SEQ ID NO:1.

In some embodiments, an ADAMTS13 variant comprises, or consists of, an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:7, and comprising one or more amino acid substitutions relative to SEQ ID NO:7.

In some embodiments, an ADAMTS13 variant comprises, or consists of, an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:8, and comprising one or more amino acid substitutions relative to SEQ ID NO:8.

In some embodiments, an ADAMTS13 variant according to the present disclosure comprises more than one amino acid substitution relative to the amino acid sequence of wildtype human ADAMTS13. In some embodiments, the ADAMTS13 variant comprises 1, 2, 3, 4, 5, 6, 7, 8, 9, 10, 11, 12, 13, 14, 15, 16, 17, 18, 19 or 20 amino acid substitutions relative to the amino acid sequence of wildtype human ADAMTS13.

In some embodiments, an ADAMTS13 variant comprises, or consists of, an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:1, and comprising 1, 2, 3, 4, 5, 6, 7, 8, 9, 10, 11, 12, 13, 14, 15, 16, 17, 18, 19 or 20 amino acid substitutions relative to SEQ ID NO:1.

In some embodiments, an ADAMTS13 variant comprises, or consists of, an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:8, and comprising 1, 2, 3, 4, 5, 6, 7, 8, 9, 10, 11, 12, 13, 14, 15, 16, 17, 18, 19 or 20 amino acid substitutions relative to SEQ ID NO:7.

In some embodiments, an ADAMTS13 variant comprises, or consists of, an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:8, and comprising 1, 2, 3, 4, 5, 6, 7, 8, 9, 10, 11, 12, 13, 14, 15, 16, 17, 18, 19 or 20 amino acid substitutions relative to SEQ ID NO 8.

The Inventors have identified ADAMTS13 variants comprising substitutions in the linker 3 (L3) region of ADAMTS13 having proteolytic activity greater than wildtype human ADAMTS13. The L3 region of ADAMTS13 is formed of positions 1131 to 1190 of SEQ ID NO:1, and is shown in SEQ ID NO:48.

In some embodiments, an ADAMTS13 variant according to the present disclosure comprises one or more amino acid substitutions relative to the amino acid sequence of wildtype human ADAMTS13 in the region corresponding to SEQ ID NO:48. In some embodiments, an ADAMTS13 variant according to the present disclosure comprises one or more amino acid substitutions relative to the amino acid sequence of wildtype human ADAMTS13 in the region corresponding to SEQ ID NO:49.

In preferred embodiments, the one or more amino acid substitutions relative to the amino acid sequence of wildtype human ADAMTS13 are associated with greater proteolytic activity for the ADAMTS13 variant as compared to the proteolytic activity of wildtype human ADAMTS13.

In some embodiments, an ADAMTS13 variant according to the present disclosure comprises one or more (e.g. 1, 2, 3, 4, 5, 6, 7, 8, 9 or 10) substitutions relative to the amino acid sequence of wildtype human ADAMTS13 at position(s) corresponding to the following positions (numbered relative to SEQ ID NO:1): A1144, A1145, A1146, P1147, P1154, P1171, P1173, P1175, P1180, P1182. In some embodiments, an ADAMTS13 variant according to the present disclosure comprises one or more (e.g. 1, 2, 3 or 4) substitutions relative to the amino acid sequence of wildtype human ADAMTS13 at position(s) corresponding to the following positions (numbered relative to SEQ ID NO:1): A1144, A1146, P1180, P1182.

In some embodiments, an ADAMTS13 variant comprises substitution relative to the amino acid sequence of wildtype human ADAMTS13 at the position corresponding to A1144. In some embodiments, an ADAMTS13 variant comprises substitution relative to the amino acid sequence of wildtype human ADAMTS13 at the position corresponding to A1145. In some embodiments, an ADAMTS13 variant comprises substitution relative to the amino acid sequence of wildtype human ADAMTS13 at the position corresponding to A1146. In some embodiments, an ADAMTS13 variant comprises substitution relative to the amino acid sequence of wildtype human ADAMTS13 at the position corresponding to P1147. In some embodiments, an ADAMTS13 variant comprises substitution relative to the amino acid sequence of wildtype human ADAMTS13 at the position corresponding to P1154. In some embodiments, an ADAMTS13 variant comprises substitution relative to the amino acid sequence of wildtype human ADAMTS13 at the position corresponding to P1171. In some embodiments, an ADAMTS13 variant comprises substitution relative to the amino acid sequence of wildtype human ADAMTS13 at the position corresponding to P1173. In some embodiments, an ADAMTS13 variant comprises substitution relative to the amino acid sequence of wildtype human ADAMTS13 at the position corresponding to P1175. In some embodiments, an ADAMTS13 variant comprises substitution relative to the amino acid sequence of wildtype human ADAMTS13 at the position corresponding to P1180. In some embodiments, an ADAMTS13 variant comprises substitution relative to the amino acid sequence of wildtype human ADAMTS13 at the position corresponding to P1182.

In some embodiments, an ADAMTS13 variant comprises substitution of an alanine residue with another hydrophobic amino acid residue relative to the amino acid sequence of wildtype human ADAMTS13. In some embodiments, an ADAMTS13 variant comprises substitution of a proline residue with another hydrophobic amino acid residue relative to the amino acid sequence of wildtype human ADAMTS13.

In some embodiments, an ADAMTS13 variant comprises substitution of an alanine residue with a valine, isoleucine, lysine, leucine, or methionine residue relative to the amino acid sequence of wildtype human ADAMTS13. In some embodiments, an ADAMTS13 variant comprises substitution of an alanine residue with a valine, isoleucine, or lysine residue relative to the amino acid sequence of wildtype human ADAMTS13.

In some embodiments, an ADAMTS13 variant comprises substitution of a proline residue with a valine, isoleucine, lysine, leucine, or methionine residue relative to the amino acid sequence of wildtype human ADAMTS13. In some embodiments, an ADAMTS13 variant comprises substitution of a proline residue with a valine, isoleucine, or lysine residue relative to the amino acid sequence of wildtype human ADAMTS13.

In some embodiments, an ADAMTS13 variant according to the present disclosure comprises one or more (e.g. 1, 2, 3, 4, 5, 6, 7, 8, 9 or 10) of the following substitutions relative to the amino acid sequence of wildtype human ADAMTS13, at corresponding position(s) (numbered relative to SEQ ID NO:1): A1144V, A1145V, A1146V, P1147V, P1154V, P1171V, P1173V, P1175V, P1180V, P1182V. In some embodiments, an ADAMTS13 variant according to the present disclosure comprises one or more (e.g. 1, 2, 3 or 4) of the following substitutions relative to the amino acid sequence of wildtype human ADAMTS13, at corresponding position(s) (numbered relative to SEQ ID NO:1): A1144V, A1146V, P1180V, P1182V. In some embodiments, an ADAMTS13 variant comprises the substitution A1144V relative to the amino acid sequence of wildtype human ADAMTS13.

In some embodiments, an ADAMTS13 variant according to the present disclosure comprises one or more (e.g. 1, 2, 3, 4, 5, 6, 7, 8, 9 or 10) of the following substitutions relative to the amino acid sequence of wildtype human ADAMTS13, at corresponding position(s) (numbered relative to SEQ ID NO:1): A1144K, A1145K, A1146K, P1147K, P1154K, P1171K, P1173K, P1175K, P1180K, P1182K. In some embodiments, an ADAMTS13 variant according to the present disclosure comprises one or more (e.g. 1, 2, 3 or 4) of the following substitutions relative to the amino acid sequence of wildtype human ADAMTS13, at corresponding position(s) (numbered relative to SEQ ID NO:1): A1144K, A1146K, P1180K, P1182K. In some embodiments, an ADAMTS13 variant comprises the substitution A1144K relative to the amino acid sequence of wildtype human ADAMTS13.

In some embodiments, an ADAMTS13 variant according to the present disclosure comprises one or more (e.g. 1, 2, 3, 4, 5, 6, 7, 8, 9 or 10) of the following substitutions relative to the amino acid sequence of wildtype human ADAMTS13, at corresponding position(s) (numbered relative to SEQ ID NO:1): A1144I, A1145I, A1146I, P1147I, P1154I, P1171I, P1173I, P1175I, P1180I, P1182I. In some embodiments, an ADAMTS13 variant according to the present disclosure comprises one or more (e.g. 1, 2, 3 or 4) of the following substitutions relative to the amino acid sequence of wildtype human ADAMTS13, at corresponding position(s) (numbered relative to SEQ ID NO:1): A1144I, A1146I, P1180I, P1182I. In some embodiments, an ADAMTS13 variant comprises the substitution A1144I relative to the amino acid sequence of wildtype human ADAMTS13.

In some embodiments, an ADAMTS13 variant comprises the substitution A1144V relative to the amino acid sequence of wildtype human ADAMTS13, at the corresponding position. In some embodiments, an ADAMTS13 variant comprises the substitution A1146V relative to the amino acid sequence of wildtype human ADAMTS13, at the corresponding position. In some embodiments, an ADAMTS13 variant comprises the substitution P1147V relative to the amino acid sequence of wildtype human ADAMTS13, at the corresponding position. In some embodiments, an ADAMTS13 variant comprises the substitution P1154V relative to the amino acid sequence of wildtype human ADAMTS13, at the corresponding position. In some embodiments, an ADAMTS13 variant comprises the substitution P1171V relative to the amino acid sequence of wildtype human ADAMTS13, at the corresponding position. In some embodiments, an ADAMTS13 variant comprises the substitution P1173V relative to the amino acid sequence of wildtype human ADAMTS13, at the corresponding position. In some embodiments, an ADAMTS13 variant comprises the substitution P1175V relative to the amino acid sequence of wildtype human ADAMTS13, at the corresponding position. In some embodiments, an ADAMTS13 variant comprises the substitution P1180V relative to the amino acid sequence of wildtype human ADAMTS13, at the corresponding position. In some embodiments, an ADAMTS13 variant comprises the substitution P1182V relative to the amino acid sequence of wildtype human ADAMTS13, at the corresponding position.

In some embodiments, an ADAMTS13 variant comprises the substitution A1144K relative to the amino acid sequence of wildtype human ADAMTS13, at the corresponding position. In some embodiments, an ADAMTS13 variant comprises the substitution A1146K relative to the amino acid sequence of wildtype human ADAMTS13, at the corresponding position. In some embodiments, an ADAMTS13 variant comprises the substitution P1147K relative to the amino acid sequence of wildtype human ADAMTS13, at the corresponding position. In some embodiments, an ADAMTS13 variant comprises the substitution P1154K relative to the amino acid sequence of wildtype human ADAMTS13, at the corresponding position. In some embodiments, an ADAMTS13 variant comprises the substitution P1171K relative to the amino acid sequence of wildtype human ADAMTS13, at the corresponding position. In some embodiments, an ADAMTS13 variant comprises the substitution P1173K relative to the amino acid sequence of wildtype human ADAMTS13, at the corresponding position. In some embodiments, an ADAMTS13 variant comprises the substitution P1175K relative to the amino acid sequence of wildtype human ADAMTS13, at the corresponding position. In some embodiments, an ADAMTS13 variant comprises the substitution P1180K relative to the amino acid sequence of wildtype human ADAMTS13, at the corresponding position. In some embodiments, an ADAMTS13 variant comprises the substitution P1182K relative to the amino acid sequence of wildtype human ADAMTS13, at the corresponding position.

In some embodiments, an ADAMTS13 variant comprises the substitution A1144I relative to the amino acid sequence of wildtype human ADAMTS13, at the corresponding position. In some embodiments, an ADAMTS13 variant comprises the substitution A1146I relative to the amino acid sequence of wildtype human ADAMTS13, at the corresponding position. In some embodiments, an ADAMTS13 variant comprises the substitution P1147I relative to the amino acid sequence of wildtype human ADAMTS13, at the corresponding position. In some embodiments, an ADAMTS13 variant comprises the substitution P1154I relative to the amino acid sequence of wildtype human ADAMTS13, at the corresponding position. In some embodiments, an ADAMTS13 variant comprises the substitution P1171I relative to the amino acid sequence of wildtype human ADAMTS13, at the corresponding position. In some embodiments, an ADAMTS13 variant comprises the substitution P1173I relative to the amino acid sequence of wildtype human ADAMTS13, at the corresponding position. In some embodiments, an ADAMTS13 variant comprises the substitution P1175I relative to the amino acid sequence of wildtype human ADAMTS13, at the corresponding position. In some embodiments, an ADAMTS13 variant comprises the substitution P1180I relative to the amino acid sequence of wildtype human ADAMTS13, at the corresponding position. In some embodiments, an ADAMTS13 variant comprises the substitution P1182I relative to the amino acid sequence of wildtype human ADAMTS13, at the corresponding position.

In some embodiments, an ADAMTS13 variant comprises an amino acid sequence according to SEQ ID NO:50, wherein the amino acid sequence is non-identical to SEQ ID NO:49.

In some embodiments, an ADAMTS13 variant comprises the amino acid sequence of SEQ ID NO:51, 136, or 146, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:51, 136, or 146, comprising V, K, or I, respectively, at the position corresponding to position 1144.

In some embodiments, an ADAMTS13 variant comprises the amino acid sequence of SEQ ID NO:52, 137, or 147, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:52, 137, or 147, comprising V, K, or I, respectively, at the position corresponding to position 1145.

In some embodiments, an ADAMTS13 variant comprises the amino acid sequence of SEQ ID NO:53, 138, or 148, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:53, 138, or 148, comprising V, K, or I, respectively, at the position corresponding to position 1146.

In some embodiments, an ADAMTS13 variant comprises the amino acid sequence of SEQ ID NO:54, 139, or 149, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:54, 139, or 149, comprising V, K, or I, respectively, at the position corresponding to position 1147.

In some embodiments, an ADAMTS13 variant comprises the amino acid sequence of SEQ ID NO:55, 140, or 150, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:55, 140, or 150, comprising V, K, or I, respectively, at the position corresponding to position 1154.

In some embodiments, an ADAMTS13 variant comprises the amino acid sequence of SEQ ID NO:56, 141, or 151, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:56, 141, or 151, comprising V, K, or I, respectively, at the position corresponding to position 1171.

In some embodiments, an ADAMTS13 variant comprises the amino acid sequence of SEQ ID NO:57, 142, or 152, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:57, 142, or 152, comprising V, K, or I, respectively, at the position corresponding to position 1173.

In some embodiments, an ADAMTS13 variant comprises the amino acid sequence of SEQ ID NO:58, 143, or 153, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:58, 143, or 153, comprising V, K, or I, respectively, at the position corresponding to position 1175.

In some embodiments, an ADAMTS13 variant comprises the amino acid sequence of SEQ ID NO:59, 144, or 154, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:59, 144, or 154, comprising V, K, or I, respectively, at the position corresponding to position 1180.

In some embodiments, an ADAMTS13 variant comprises the amino acid sequence of SEQ ID NO:60, 145, or 155, or an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:60, 145, or 155, comprising V, K, or I, respectively, at the position corresponding to position 1182.

One known ADAMTS13 variant, known as a gain of function (GoF) ADAMTS13 variant (R568K/F592Y/R660K/Y661F/Y665F), has previously been shown to have disrupt autoinhibition and enhance proteolytic activity as compared to wild-type human ADAMTS13, and is described in Jian et al., Blood (2012) 119: 3836-3843 (hereby incorporated by reference in its entirety). The R568K/F592Y/R660K/Y661F/Y665F variant amino acid substitutions are provided in the spacer region of ADAMTS13. Whilst this known GoF variant has higher activity than wild type ADAMTS13, it has lower proteolytic activity than variants of the present disclosure.

In some embodiments, an ADAMTS13 variant of the present disclosure is non-identical to an ADAMTS13 variant disclosed in Jian et al., Blood (2012) 119: 3836-3843. In some embodiments, an ADAMTS13 variant of the present disclosure does not comprise, or consist of, the amino acid sequence of SEQ ID NO:71.

In some embodiments, an ADAMTS13 variant comprises one or more (e.g. 1, 2, 3, 4 or 5) substitutions relative to the amino acid sequence of wildtype human ADAMTS13 at position(s) corresponding to the following positions (numbered relative to SEQ ID NO:1): R568, F592, R660, Y661, Y665.

In some embodiments, an ADAMTS13 variant comprises one or more (e.g. 1, 2, 3, 4 or 5) of the following substitutions relative to the amino acid sequence of wildtype human ADAMTS13, at corresponding position(s) (numbered relative to SEQ ID NO:1): R568K, F592Y, R660K, Y661F, Y665F.

In some embodiments, an ADAMTS13 variant comprises the amino acid sequence of SEQ ID NO:13, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:13.

In some embodiments, an ADAMTS13 variant comprises the amino acid sequence of SEQ ID NO:14, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:14.

In some embodiments, an ADAMTS13 variant comprises the amino acid sequence of SEQ ID NO:15, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:15.

In some embodiments, an ADAMTS13 variant comprises the amino acid sequence of SEQ ID NO:16, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:16.

In some embodiments, an ADAMTS13 variant comprises the amino acid sequence of SEQ ID NO:17, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:17.

In some embodiments, an ADAMTS13 variant comprises the amino acid sequence of SEQ ID NO:18, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:18.

In some embodiments, an ADAMTS13 variant comprises the amino acid sequence of SEQ ID NO:19, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:19.

In some embodiments, an ADAMTS13 variant comprises the amino acid sequence of SEQ ID NO 20, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:20.

In some embodiments, an ADAMTS13 variant comprises the amino acid sequence of SEQ ID N021, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:21.

In some embodiments, an ADAMTS13 variant comprises the amino acid sequence of SEQ ID NO:22, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:22.

In some embodiments, an ADAMTS13 variant comprises the amino acid sequence of SEQ ID NO:23, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:23.

In some embodiments, an ADAMTS13 variant comprises the amino acid sequence of SEQ ID NO:24, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:24.

In some embodiments, an ADAMTS13 variant comprises the amino acid sequence of SEQ ID NO:25, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:25.

In some embodiments, an ADAMTS13 variant comprises the amino acid sequence of SEQ ID N026, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:26.

In some embodiments, an ADAMTS13 variant comprises, or consists of, the amino acid sequence of SEQ ID NO:61, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:61.

In some embodiments, an ADAMTS13 variant comprises, or consists of, the amino acid sequence of SEQ ID NO:62, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:62.

In some embodiments, an ADAMTS13 variant comprises, or consists of, the amino acid sequence of SEQ ID NO:63, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:63.

In some embodiments, an ADAMTS13 variant comprises, or consists of, the amino acid sequence of SEQ ID NO:64, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:64.

In some embodiments, an ADAMTS13 variant comprises, or consists of, the amino acid sequence of SEQ ID NO:65, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:65.

In some embodiments, an ADAMTS13 variant comprises, or consists of, the amino acid sequence of SEQ ID NO:66, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:66.

In some embodiments, an ADAMTS13 variant comprises, or consists of, the amino acid sequence of SEQ ID NO:67, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:67.

In some embodiments, an ADAMTS13 variant comprises, or consists of, the amino acid sequence of SEQ ID NO:68, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:68.

In some embodiments, an ADAMTS13 variant comprises, or consists of, the amino acid sequence of SEQ ID NO:69, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:69.

In some embodiments, an ADAMTS13 variant comprises, or consists of, the amino acid sequence of SEQ ID NO:70, or an amino acid sequence having at least 60%, preferably one of 65%, 70%, 75%, 80%, 85%, 90%, 91%, 92%, 93%, 94%, 95%, 96%, 97%, 98%, 99% or 100% amino acid sequence identity to SEQ ID NO:70.

### Functional Properties of the ADAMTS13 Variants

The ADAMTS13 variants of the present disclosure may be characterised by reference to certain functional properties.

In some embodiments, an ADAMTS13 variant described herein may display one or more of the following properties:
proteolytic activity, e.g. VWF-cleaving activity;
increased proteolytic activity as compared to wildtype human ADAMTS13; increased proteolytic activity as compared to the R568K/F592Y/R660K/Y661F/Y665F ADAMTS13 variant;
inhibition of VWF-mediated platelet capture, e.g. under arterial shear stress;
increased inhibition of VWF-mediated platelet capture as compared to wildtype human ADAMTS13;
increased inhibition of VWF-mediated platelet capture as compared to the R568K/F592Y/R660K/Y661F/Y665F ADAMTS13 variant;
inhibition of thrombosis;
increased inhibition of thrombosis as compared to wildtype human ADAMTS13;
increased inhibition of thrombosis as compared to the R568K/F592Y/R660K/Y661F/Y665F ADAMTS13 variant;
inhibition of blood clotting/coagulation;
increased inhibition of blood clotting/coagulation as compared to wildtype human ADAMTS13;
increased inhibition of blood clotting/coagulation as compared to the R568K/F592Y/R660K/Y661F/Y665F ADAMTS13 variant;
inhibition of inflammation, e.g. in an in vivo model of inflammation;
increased inhibition of inflammation as compared to wildtype human ADAMTS13;
increased inhibition of inflammation as compared to the R568K/F592Y/R660K/Y661F/Y665F ADAMTS13 variant.

As explained hereinabove, ADAMTS13 cleaves VWF, at the peptide bond formed between positions Y1605 and M1606 (numbering relative to SEQ ID NO:27), within the VWFA2 domain of VWF. The present disclosure is particularly concerned with variants of ADAMTS13 having improved/increased proteolytic activity as compared to wildtype human ADAMTS13 (e.g. a polypeptide comprising, or consisting of, the amino acid sequence of SEQ ID NO:1, 7 or 8).

Proteolytic activity refers to the lysis of peptides/polypeptides, producing smaller peptides/polypeptides and/or the constituent amino acids thereof. Proteolysis may involve hydrolysis of peptide bonds between amino acids of a peptide/polypeptide. Proteolytic activity is the enzymatic activity of an enzyme performing proteolysis. Proteolytic activity is determined by the amount of substrate broken down (or amount of substrate hydrolysis) by a pre-determined amount of protease over a pre-determined time period. Proteolytic activity can be measured using any method known in the art.

ADAMTS13 variants having improved proteolytic activity relative to wildtype human ADAMTS13 may be described as having greater/higher/Improved/Increased metalloprotease activity, or may be described as having greater/higher/improved/increased VWF-cleaving activity relative to wildtype human ADAMTS13.

A given variant of ADAMTS13 may be evaluated for proteolytic activity in a suitable in vitro assay. Such an assay may comprise contacting a substrate for cleavage by ADAMTS13 with the ADAMTS13 variant under conditions suitable for cleavage of the substrate by ADAMTS13, and analysing the sample for products of cleavage and/or for uncleaved substrate after a sufficient period of time for cleavage of the substrate to have occurred.

An example of a suitable assay of such activity is the assay evaluating cleavage of the FRETS-VWF73 molecule, as described in South et al. Proc Natl Acad Sci USA. (2014) 111(52): 18578-18583, which is hereby incorporated by reference In Its entirety. FRETS-VWF73 Is a VWF fragment comprising VWFA2 domain residues 1596 to 1668 encompassing the ADAMTS13 cleavage site.

Briefly, ADAMTS13 variants may be diluted to a concentration of 0.3 nM in 5 mM Bis-Tris pH 6.0, 25 mM CaCl₂, and 0.005% Tween-20, in white 96-well plates (Nunc). Purified VWF D4-CK fragment may be added to a final concentration of 20-60 nM prior to a 45 min preincubation at 37° C., or the assay may performed in the absence of added purified VWF D4-CK fragment. The reaction may be initiated by the addition of an equal volume of 4 μM FRETS-VWF73 substrate (Peptanova). Fluorescence (excitation, 340 nm; emission, 460 nm) may be measured at 30° C. at 1 min intervals for 1 h using an appropriate plate reader (e.g. Fluostar Omega plate reader (BMG Labtech)). Fluorescence measurements may be compared to values obtained for wildtype human ADAMTS13.

In some embodiments, an ADAMTS13 variant according to the present disclosure displays a level of proteolytic activity which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.1 times, ≥3.2 times, ≥3.3 times, ≥3.4 times, ≥3.5 times, ≥3.6 times, ≥3.7 times, ≥3.8 times, ≥3.9 times, ≥4 times, ≥4.1 times, ≥4.2 times, ≥4.3 times, ≥4.4 times, ≥4.5 times, ≥4.6 times, ≥4.7 times, ≥4.8 times, ≥4.9 times or ≥5 times the level proteolytic activity determined for wildtype human ADAMTS13 (e.g. a polypeptide comprising, or consisting of, the amino acid sequence of SEQ ID NO:1, 7 or 8) in the same assay.

In preferred embodiments, an ADAMTS13 variant according to the present disclosure may display a level of proteolytic activity which is greater than 2.5 times, e.g. one of ≥2.6 times, ≥2.7 times, ≥2.8 times, ≥2.9 times, ≥3 times, ≥3.1 times, ≥3.2 times, ≥3.3 times, ≥3.4 times, ≥3.5 times, ≥3.6 times, ≥3.7 times, ≥3.8 times, ≥3.9 times, ≥4 times, ≥4.1 times, ≥4.2 times, ≥4.3 times, ≥4.4 times, ≥4.5 times, ≥4.6 times, ≥4.7 times, ≥4.8 times, ≥4.9 times or ≥5 times the level proteolytic activity determined for wildtype human ADAMTS13 (e.g. a polypeptide comprising, or consisting of, the amino acid sequence of SEQ ID NO:1, 7 or 8) in the same assay.

In some embodiments, an ADAMTS13 variant according to the present disclosure has improved/increased proteolytic activity as compared to the ADAMTS13 R568K/F592Y/R660K/Y661F/Y665F variant described in Jian et al., Blood (2012) 119: 3836-3843. In some embodiments, an ADAMTS13 variant has improved proteolytic activity as compared to a polypeptide, comprising or consisting of, the amino acid sequence of SEQ ID NO:71.

In some embodiments, an ADAMTS13 variant according to the present disclosure may display a level of proteolytic activity which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.1 times, ≥3.2 times, ≥3.3 times, ≥3.4 times, ≥3.5 times, ≥3.6 times, ≥3.7 times, ≥3.8 times, ≥3.9 times, ≥4 times, ≥4.1 times, ≥4.2 times, ≥4.3 times, ≥4.4 times, ≥4.5 times, ≥4.6 times, ≥4.7 times, ≥4.8 times, ≥4.9 times or ≥5 times the level proteolytic activity determined for the ADAMTS13 R568K/F592Y/R660K/Y661F/Y665F variant (e.g. a polypeptide comprising, or consisting of, the amino acid sequence of SEQ ID NO:71) in the same assay.

In preferred embodiments, an ADAMTS13 variant according to the present disclosure may display a level of proteolytic activity which is greater than 2 times, e.g. one of ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.1 times, ≥3.2 times, ≥3.3 times, ≥3.4 times, ≥3.5 times, ≥3.6 times, ≥3.7 times, ≥3.8 times, ≥3.9 times, ≥4 times, ≥4.1 times, ≥4.2 times, ≥4.3 times, ≥4.4 times, ≥4.5 times, ≥4.6 times, ≥4.7 times, ≥4.8 times, ≥4.9 times or ≥5 times the level proteolytic activity determined for the ADAMTS13 R568K/F592Y/R660K/Y661F/Y665F variant (e.g. a polypeptide comprising, or consisting of, the amino acid sequence of SEQ ID NO:71) in the same assay.

In some embodiments, an ADAMTS13 variant according to the present disclosure displays improved/increased inhibition of VWF-mediated platelet capture as compared to wildtype human ADAMTS13, and/or the ADAMTS13 R568K/F592Y/R660K/Y661F/Y665F variant described in Jian et al., Blood (2012) 119: 3836-3843.

A given variant of ADAMTS13 may be evaluated for its ability to inhibit VWF-mediated platelet capture under arterial shear stress, and/or for the extent to which it inhibits VWF-mediated platelet capture under arterial shear stress, in a suitable in vitro assay. An example of such an assay is described in Example 1 herein.

The ability of ADAMTS13 variants to inhibit VWF-mediated platelet capture under arterial shear stress can be assessed through a FRETS-VWF73 assay. FRETS-VWF73 assays of ADAMTS13-mediated proteolysis of VWF are described in South et al., 2018.

One way of performing the assay is as follows: Coat Vena8 Fluoro+ biochips (Cellix) with 200 μg/ml collagen type III (Southern Biotech) and block with 1% BSA, 1 mg/ml glucose in HEPES buffer. Combine washed platelets with red blood cells, and treat with 100 nM PGE1 and 75 mU/ml Apyrase, to prevent platelet activation, before labelling platelets 10 μM DiOC6. Supplement platelets with 10 μg/ml multimeric plasma VWF and perfuse over the collagen surface at a constant shear rate of 1500 s-1 (at which platelet capture is VWF dependent) for 5 minutes. Adhesion of labelled platelets can be visualised by fluorescence imaging at 250 ms intervals using a 20× objective and analysed using Slidebook software to determine platelet coverage (%) at 270 seconds. To determine the effect of ADAMTS13 on platelet capture the assay can be performed in the presence of WT or variant ADAMTS13 at a range of concentrations. EC50 values can be determined by dose-response curves.

In some embodiments, an ADAMTS13 variant according to the present disclosure inhibits VWF-mediated platelet capture to a level which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the level to which VWF-mediated platelet capture is inhibited by wildtype human ADAMTS13 (e.g. a polypeptide comprising, or consisting of, the amino acid sequence of SEQ ID NO:1, 7 or 8) In the same assay.

In some embodiments, an ADAMTS13 variant according to the present disclosure inhibits VWF-mediated platelet capture to a level which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the level to which VWF-mediated platelet capture is inhibited by the ADAMTS13 R568K/F592Y/R660K/Y661F/665F variant (e.g. a polypeptide comprising, or consisting of, the amino acid sequence of SEQ ID NO:71) in the same assay.

In some embodiments, an ADAMTS13 variant according to the present disclosure displays improved/increased inhibition of thrombosis as compared to wildtype human ADAMTS13, and/or the ADAMTS13 R568K/F592Y/R660K/Y661F/Y665F variant described in Jian et al., Blood (2012) 119: 3836-3843.

A given variant of ADAMTS13 may be evaluated for its ability to inhibit thrombosis, and/or for the extent to which it inhibits thrombosis, in a suitable in vitro or in vivo assay.

One such appropriate in vitro assay is performed as follows: platelet-rich plasma or whole blood can be perfused over a pro-thrombotic surface (collagen and/or tissue factor coated) in a microfluidic perfusion chamber. Using fluorescently labelled donor platelets and/or fluorescently labelled fibrinogen allows thrombus formation to be monitored microscopically in real time. Other methods are known in the art.

One such appropriate in vivo assay is performed as follows: application of FeCl₃ to the exposed middle cerebral artery or the mesenteric arteries of the cremaster muscle in mice or rats. Can be performed in the presence of fluorescently labelled donor platelets and leukocytes and/or labelled fibrinogen. Thrombus formation can be visualised in situ using 2-photon microscopy. Other methods are known in the art.

In some embodiments, an ADAMTS13 variant according to the present disclosure inhibits thrombosis to a level which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the level to which thrombosis is inhibited by wildtype human ADAMTS13 (e.g. a polypeptide comprising, or consisting of, the amino acid sequence of SEQ ID NO:1, 7 or 8) in the same assay.

In some embodiments, an ADAMTS13 variant according to the present disclosure inhibits thrombosis to a level which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the level to which thrombosis is inhibited by the ADAMTS13 R568K/F592Y/R660K/Y661F/Y685F variant (e.g. a polypeptide comprising, or consisting of, the amino acid sequence of SEQ ID NO:71) in the same assay.

In some embodiments, an ADAMTS13 variant according to the present disclosure displays improved/increased inhibition of blood clotting/coagulation as compared to wildtype human ADAMTS13, and/or the ADAMTS13 R568K/F592Y/R660K/Y661F/Y665F variant described in Jian et al., Blood (2012) 119: 3836-3843.

A given variant of ADAMTS13 may be evaluated for its ability to inhibit blood clotting/coagulation, and/or for the extent to which it inhibits blood clotting/coagulation, in a suitable in vitro or in vivo assay.

One such appropriate in vitro assay is performed as follows: blood clotting can be measured simply using a turbidity assay. The absorbance of blood (or more often platelet rich or platelet poor plasma) is measured at 405 nm and increases upon addition of thrombin or tissue factor, as fibrinogen is converted to fibrin forming a clot. Other methods are known in the art.

The time taken for cessation of bleeding, following transverse amputation of the tall of a rodent, can be used as an in vivo assay. Other methods are known in the art.

In some embodiments, an ADAMTS13 variant according to the present disclosure inhibits blood clotting/coagulation to a level which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the level to which blood clotting/coagulation is inhibited by wildtype human ADAMTS13 (e.g. a polypeptide comprising, or consisting of, the amino acid sequence of SEQ ID NO:1, 7 or 8) in the same assay.

In some embodiments, an ADAMTS13 variant according to the present disclosure inhibits blood clotting/coagulation to a level which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the level to which blood clotting/coagulation is inhibited by the ADAMTS13 R568K/F592Y/R660K/Y661F/Y665F variant (e.g. a polypeptide comprising, or consisting of, the amino acid sequence of SEQ ID NO:71) in the same assay.

In some embodiments, an ADAMTS13 variant according to the present disclosure displays improved/increased inhibition of inflammation as compared to wildtype human ADAMTS13, and/or the ADAMTS13 R568K/F592Y/R660K/Y661F/Y665F variant described in Jian et al., Blood (2012) 119: 3836-3843.

A given variant of ADAMTS13 may be evaluated for its ability to inhibit inflammation, and/or for the extent to which it inhibits inflammation, in a suitable in vitro or in vivo assay. An example of such an assay is described in Example 1 herein.

As described in the examples, magnetic resonance imaging (MRI) scans can be used to assess levels of inflammation. Other methods of assessing inflammation are known in the art.

In the examples, the efficacy of ADAMTS13 variants is defined as a reduction in R2* in MGE MRI scans (Indicating reduced vascular inflammation in the brain) and/or reduced reactive VWF species in the plasma.

In some embodiments, an ADAMTS13 variant according to the present disclosure inhibits inflammation to a level which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the level to which inflammation is inhibited by wildtype human ADAMTS13 (e.g. a polypeptide comprising, or consisting of, the amino acid sequence of SEQ ID NO:1, 7 or 8) in the same assay.

In some embodiments, an ADAMTS13 variant according to the present disclosure inhibits inflammation to a level which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times or ≤0.1 times the level to which inflammation is inhibited by the ADAMTS13 R568K/F592Y/R660K/Y661F/Y665F variant (e.g. a polypeptide comprising, or consisting of, the amino acid sequence of SEQ ID NO:71) in the same assay.

### Additional Sequences and Linkers

In some embodiments, ADAMTS13 variants according to the present disclosure may additionally comprise one or more further amino acids or sequences of amino acids.

In some embodiments, an ADAMTS13 variant according to the present disclosure comprise one or more linker sequences, e.g. between amino acid sequences of the ADAMTS13 variant. A linker sequence may be provided at one or both ends of one or more of a specified amino acid sequence of the ADAMTS13 variant.

Linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the linker sequence comprises at least one glycine residue and/or at least one serine residue. In some embodiments the linker sequence consists of glycine and serine residues. In some embodiments, the linker sequence comprises one or more copies (e.g. in tandem) of the sequence motif G₄S. In some embodiments, the linker sequence has a length of 1-2, 1-3, 1-4, 1-5, 1-10, 1-15, 1-20, 1-25, or 1-30 amino acids.

An ADAMTS13 variant may comprise amino acid sequence(s) to facilitate expression, folding, trafficking, processing, purification or detection of the polypeptide. For example, the ADAMTS13 variants may comprise a sequence encoding a His, (e.g. 6×His), Myc, GST, MBP, FLAG, HA, E, or Biotin tag, optionally at the N- or C-terminus of the polypeptide. By way of illustration, the ADAMTS13 variants exemplified herein comprise a C-terminal Myc/6×His tag.

In some embodiments, an ADAMTS13 variant further comprises a detectable moiety, e.g. a fluorescent, luminescent, immuno-detectable, radio, chemical, nucleic acid or enzymatic label.

In some embodiments, an ADAMTS13 variant further comprises a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides.

The signal peptide may be present at the N-terminus of the polypeptide, and may be present in the newly-synthesised polypeptide. The signal peptide provides for efficient trafficking and secretion of the polypeptide. Signal peptides are often removed by cleavage, and thus are not comprised in the mature polypeptide secreted from the cell expressing the polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176). In some embodiments, the signal peptide comprises or consists of an amino acid sequence having at least 60%, preferably one of 65%, 70%, 75%, 80%, 85%, 90%, 91%, 92%, 93%, 94%, 95%, 96%, 97%, 98%, 99% or greater amino acid sequence identity to SEQ ID NO:5.

### Labels and Conjugates

In some embodiments, an ADAMTS13 variant according to the present disclosure additionally comprises a label or conjugate.

In some embodiments, the detectable moiety may be a fluorescent label, phosphorescent label, luminescent label, immuno-detectable label (e.g. an epitope tag), radiolabel, chemical, nucleic acid or enzymatic label. The ADAMTS13 variant may be covalently or non-covalently labelled with the detectable moiety.

Fluorescent labels include e.g. fluorescein, rhodamine, allophycocyanin, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Iodine¹²³, Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rhenium¹⁰⁵, Scandium⁴⁷, Tellurium^{121m}, Tellurium^{122m}, Tellurium^{125m}, Thulium¹⁶⁵, Thulium¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, Isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers. Enzymatic labels include e.g. peroxidase, alkaline phosphatase, glucose oxidase, beta-galactosidase and luciferase.

In some embodiments, the ADAMTS13 variant is conjugated to a chemical moiety. The chemical moiety may be a moiety for providing a therapeutic effect. Antibody-drug conjugates are reviewed e.g. in Parslow et al., Biomedicines. 2016 September; 4(3):14. In some embodiments, the chemical moiety may be a drug moiety (e.g. a cytotoxic agent). In some embodiments, the drug moiety may be a chemotherapeutic agent. In some embodiments, the drug moiety is selected from calicheamicin, DM1, DM4, monomethylauristatin E (MMAE), monomethylauristatin F (MMAF), SN-38, doxorubicin, duocarmycin, D6.5 and PBD.

### Nucleic Acids, Cells, Compositions and Kits

The present disclosure provides a nucleic acid encoding a polypeptide or according to the present disclosure. In some embodiments the nucleic acid comprises or consists of DNA and/or RNA.

The present disclosure also provides a vector comprising the nucleic acid according to the present disclosure.

Nucleic acids and vectors according to the present disclosure may be provided in purified or isolated form, i.e. from other nucleic acid, or naturally-occurring biological material.

The nucleotide sequence may be contained in a vector, e.g. an expression vector. A “vector” as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known In the art may be used to express a peptide or polypeptide from a vector according to the present disclosure.

The term “operably linked” may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide/polypeptide.

The nucleic acid and/or vector according to the present disclosure is preferably provided for introduction into a cell. Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in its entirety.

In some embodiments, the vector may be a eukaryotic vector, e.g. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression. In some embodiments, the viral vector may be a lentiviral, retroviral, adenoviral, or Herpes Simplex Virus vector. In some embodiments, the lentiviral vector may be pELNS, or may be derived from pELNS.

In some embodiments, the nucleic acid according to the present disclosure comprises, or consists of, a nucleic acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NOs:86, 87, 88, 89, 90, 91, 92, 93, 94 or 95 or to a nucleic acid sequence encoding the same amino acid sequence as one of SEQ ID NOs:86, 87, 88, 89, 90, 91, 92, 93, 94 or 95 as a result of codon degeneracy.

### Cells Comprising/Expressing ADAMTS13 Variants

The present disclosure also provides a cell comprising or expressing an ADAMTS13 variant according to the present disclosure. Also provided is a cell comprising or expressing a nucleic acid or a vector according to the present disclosure. The cell comprising or expressing an ADAMTS13 variant, nucleic acid or vector according to the present disclosure may secrete an ADAMTS13 variant according to the present disclosure. That is, expression of an ADAMTS13 variant, nucleic acid or vector may result in the soluble production of an ADAMTS13 variant according of the present disclosure from the cell.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell is, or is derived from, a cell type commonly used for the expression of polypeptides for use in therapy in humans. Exemplary cells are described e.g. In Kunert and Reinhart, Appl Microbiol Biotechnol. (2016) 100:3451-3461 (hereby incorporated by reference in its entirety), and include e.g. CHO, HEK 293, PER.C6, NS0 and BHK cells.

The present disclosure also provides a method for producing a cell comprising a nucleic acid(s) or vector(s) according to the present disclosure, comprising introducing a nucleic acid or a vector according to the present disclosure into a cell. In some embodiments, introducing an isolated nucleic acid(s) or vector(s) according to the present disclosure into a cell comprises transformation, transfection, electroporation or transduction (e.g. retroviral transduction).

The present disclosure also provides a method for producing a cell expressing/comprising an ADAMTS13 variant according to the present disclosure, comprising introducing a nucleic acid or a vector according to the present disclosure in a cell. In some embodiments, the methods additionally comprise culturing the cell under conditions suitable for expression of the nucleic acid(s) or vector(s) by the cell. In some embodiments, the methods are performed in vitro.

The present disclosure also provides cells obtained or obtainable by the methods according to the present disclosure.

### Producing the ADAMTS13 Variants

ADAMTS13 variants according to the present disclosure may be prepared according to methods for the production of polypeptides known to the skilled person.

Polypeptides may be prepared by chemical synthesis, e.g. liquid or solid phase synthesis. For example, peptides/polypeptides can by synthesised using the methods described in, for example, Chandrudu et al., Molecules (2013), 18: 4373-4388, which is hereby incorporated by reference in its entirety.

Alternatively, polypeptides may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press, 2012, and in Nat Methods. (2008); 5(2): 135-146 both of which are hereby incorporated by reference in their entirety.

For recombinant production according to the present disclosure, any cell suitable for the expression of polypeptides may be used. The cell may be a prokaryote or eukaryote. In some embodiments the cell is a prokaryotic cell, such as a cell of archaea or bacteria. In some embodiments the bacteria may be Gram-negative bacteria such as bacteria of the family Enterobacteriaceae, for example Escherichia coli. In some embodiments, the cell is a eukaryotic cell such as a yeast cell, a plant cell, Insect cell or a mammalian cell, e.g. a cell described hereinabove.

In some cases, the cell is not a prokaryotic cell because some prokaryotic cells do not allow for the same folding or post-translational modifications as eukaryotic cells. In addition, very high expression levels are possible In eukaryotes and proteins can be easier to purify from eukaryotes using appropriate tags. Specific plasmids may also be utilised which enhance secretion of the protein into the media.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the polypeptide(s) of interest may be isolated. Any suitable method for separating proteins from cells known in the art may be used. In order to isolate the polypeptide, it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for isolating/purifying polypeptides are known in the art, for example ion exchange chromatography and size chromatography. The polypeptide may also be affinity-purified using an appropriate binding partner for a molecular tag on the polypeptide (e.g. a His, FLAG, Myc, GST, MBP, HA, E, or Biotin tag). These techniques be used as an alternative to precipitation, or may be performed subsequently to precipitation. In some cases it may further be desired to process the polypeptide, e.g. to remove a sequence of amino acids, molecular tag, moiety, etc.

In some embodiments, treatment is with an appropriate endopeptidase for the cleavage and removal of an amino acid sequence. In some embodiments, treatment is with an enzyme to remove the moiety of interest. In some embodiments, the polypeptide is treated to remove glycans (i.e. the polypeptide is deglycosylated), e.g. by treatment with a glycosidase such as with a Peptide:N-glycosidase (PNGase).

Other methods for distinguishing different proteins are known in the art, for example ion exchange chromatography and size chromatography. These may be used as an alternative to precipitation or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

In some embodiments, the production of the polypeptide occurs in vivo, e.g. after introduction to the host of a cell comprising a nucleic acid or vector encoding an ADAMTS13 variant of the present disclosure, or following introduction into a cell of the host of a nucleic acid or vector encoding an ADAMTS13 variant of the present disclosure. In such embodiments, the polypeptide is transcribed, translated and post-translationally processed to the mature polypeptide. In some embodiments, the polypeptide is produced in situ at the desired location in the host.

### Compositions

The present disclosure also provides compositions comprising the ADAMTS13 variants, nucleic acids, expression vectors and cells described herein.

The ADAMTS13 variants, nucleic acids, expression vectors and cells described herein may be formulated as pharmaceutical compositions or medicaments for clinical use and may comprise a pharmaceutically acceptable carrier, diluent, excipient or adjuvant. The composition may be formulated for topical, parenteral, systemic, Intracavitary, Intravenous, Intra-arterial, Intramuscular, Intrathecal, Intraocular, intraconjunctival, intratumoral, subcutaneous, intradermal, intrathecal, oral or transdermal routes of administration which may include injection or infusion.

Suitable formulations may comprise an ADAMTS13 variant in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments the composition is formulated for injection or infusion, e.g. into a blood vessel or tissue/organ of interest.

The present disclosure also provides methods for the production of pharmaceutically useful compositions, such methods of production may comprise one or more steps selected from: producing an ADAMTS13 variant, nucleic acid, expression vector or cell described herein; isolating an ADAMTS13 variant, nucleic acid, expression vector or cell described herein; and/or mixing an ADAMTS13 variant, nucleic acid, expression vector or cell described herein with a pharmaceutically acceptable carrier, adjuvant, excipient or diluent.

For example, a further aspect the present disclosure provides a method of formulating or producing a medicament or pharmaceutical composition for use in the treatment of a disease/condition (e.g. a disease described hereinbelow), the method comprising formulating a pharmaceutical composition or medicament by mixing an ADAMTS13 variant, nucleic acid, expression vector or cell described herein with a pharmaceutically acceptable carrier, adjuvant, excipient or diluent.

In aspects and embodiments of the present disclosure, an ADAMTS13 variant may be provided in a composition comprising particular chemical constituents in specified concentrations/proportions.

In some embodiments, an ADAMTS13 variant is provided in a buffer. As used herein, a “buffer” refers to a buffered solution that resists changes in pH by the action of its acid-base conjugate components. A buffer of the present disclosure preferably has a pH in the range from about 4.5 to about 7.0, preferably from about 5.0 to about 6.5. Examples of buffers that will control the pH in this range include acetate, histidine, histidine-arginine, histidine-methionine and other organic acid buffers.

### Kits

In some aspects of the present disclosure a kit of parts is provided. In some embodiments the kit may have at least one container having a predetermined quantity of an ADAMTS13 variant, nucleic acid, expression vector, cell or composition described herein.

In some embodiments, the kit may comprise materials for producing an ADAMTS13 variant, nucleic acid, expression vector, cell or composition described herein. For example, the kit may comprise materials for modifying a cell to express or comprise an ADAMTS13 variant, nucleic acid, expression vector, according to the present disclosure, or materials for introducing into a cell the nucleic acid, expression vector, according to the present disclosure.

The kit may provide an ADAMTS13 variant, nucleic acid, expression vector, cell or composition together with instructions for administration to a patient in order to treat or prevent a specified disease/condition, e.g. a disease/condition described hereinbelow.

In some embodiments the kit may further comprise at least one container having a predetermined quantity of another therapeutic/prophylactic agent (e.g. a therapeutic/prophylactic agent for the treatment/prevention of a disease/condition described herein). In such embodiments, the kit may also comprise a second medicament or pharmaceutical composition such that the two medicaments or pharmaceutical compositions may be administered simultaneously or separately such that they provide a combined treatment/prevention for the specific disease or condition.

### Therapeutics and Prophylactic Applications

The ADAMTS13 variants, nucleic acids, vectors, cells and pharmaceutical compositions described herein find use in therapeutic and prophylactic methods.

The present disclosure provides an ADAMTS13 variant, nucleic acid, vector, cell or pharmaceutical composition according to the present disclosure for use in a method of medical treatment or prophylaxis. The present disclosure also provides the use of an ADAMTS13 variant, nucleic acid, vector, cell or pharmaceutical composition according to the present disclosure in the manufacture of a medicament for treating or preventing a disease or condition. The present disclosure also provides a method of treating or preventing a disease or condition, comprising administering to a subject a therapeutically or prophylactically effective amount of an ADAMTS13 variant, nucleic acid, vector, cell or pharmaceutical composition according to the present disclosure.

The methods may be effective to reduce the development or progression of a disease/condition, alleviation of the symptoms of a disease/condition or reduction in the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the symptoms of the disease/condition or reduction in some other correlate of the severity/activity of the disease/condition. In some embodiments the methods may prevent development of the disease/condition a later stage (e.g. a chronic stage).

It will be appreciated that the articles of the present disclosure may be used for the treatment/prevention of any disease/condition that would derive therapeutic or prophylactic benefit from a reduction in the level and/or activity of VWF, and/or the level and/or activity of a complex comprising VWF (e.g. a multimeric complex, e.g. UL-VWF multimers). For example, the disease/condition may be a disease/condition in which VWF and/or a complex comprising VWF are pathologically implicated, e.g. a disease/condition in which an increased level of VWF and/or a complex comprising VWF is positively associated with the onset, development or progression of the disease/condition, and/or severity of one or more symptoms of the disease/condition, or for which an increased level of VWF and/or a complex comprising VWF is a risk factor for the onset, development or progression of the disease/condition.

In particular, the articles of the present disclosure may be used for the treatment/prevention of any disease/condition that would derive therapeutic or prophylactic benefit from inhibition of thrombosis. For example, the disease/condition may be a disease/condition in which thrombosis is pathologically implicated, e.g. a disease/condition in which thrombosis is positively associated with the onset, development or progression of the disease/condition, and/or severity of one or more symptoms of the disease/condition, or for which thrombosis is a risk factor for the onset, development or progression of the disease/condition.

It will also be appreciated that the articles of the present disclosure may be used for the treatment/prevention of any disease/condition that would derive therapeutic or prophylactic benefit from an increased level of ADAMTS13 or ADAMTS13 proteolytic activity. For example, the disease/condition may be a disease/condition in deficiency/insufficiency of ADAMTS13 or ADAMTS13 proteolytic activity is pathologically implicated, e.g. a disease/condition In which a decrease In the level of ADAMTS13 and/or a decrease in the level of ADAMTS13 proteolytic activity is positively associated with the onset, development or progression of the disease/condition, and/or severity of one or more symptoms of the disease/condition, or for which a decrease in the level of ADAMTS13 and/or a decrease in the level of ADAMTS13 proteolytic activity is a risk factor for the onset, development or progression of the disease/condition.

In some embodiments, the disease/condition to be treated/prevented in accordance with the present disclosure is a disease/condition characterised by an increase in the level of VWF and/or a complex comprising VWF, e.g. as compared to the level of VWF and/or a complex comprising VWF in the absence of the disease/condition.

In particular, the ADAMTS13 variants, nucleic acids, vectors, cells and pharmaceutical compositions according to the present disclosure find use to treat or prevent diseases/conditions associated with VWF and/or a complex comprising VWF, e.g. diseases associated with elevated levels or activity of VWF and/or a complex comprising VWF.

In accordance with various aspects of the present disclosure, a method of treating and/or preventing a disease/condition described herein may comprise one or more of the following: reducing the level or activity of VWF, reducing the level or activity of a complex comprising VWF; reducing the level of a correlate of the level or activity of VWF, reducing the level of a correlate of the level or activity of a complex comprising VWF, reducing or inhibiting thrombosis; reducing or inhibiting blood clotting/coagulation; reducing or inhibiting inflammation; increasing the level of ADAMTS13 proteolytic activity, Increasing proteolysis of VWF and/or complexes comprising VWF.

Thrombotic thrombocytopenic purpura (TTP) is characterised by a severe deficiency In ADAMTS13 either through acquired auto-antibodies that inhibit function or enhance clearance (idiopathic) or through loss of functional mutations in the ADAMTS13 gene (congenital). Acute episodes feature disseminated UL-VWF rich microthrombi leading to multiple organ ischemia and mortality In 20% of patients with a high risk of reoccurring relapses in those who survive. Current therapies include time-consuming plasma infusions which replenish functional ADAMTS13 levels but run the risk of introducing further complications including severe allergic reactions, volume overload and harmful blood-borne diseases (Sadler 2008; Kremer Hovinga et al. 2017). Recombinant human ADAMTS13 (Bax930—NCT03393975) is shown to have a comparable half-life and pharmacokinetic profile to plasma-derived ADAMTS13 and is currently In phase 3 clinical trials for patients with congenital TTP (Scully et al. 2017). However, using an improved ADAMTS13 variant in this setting has the potential to further reduce dosage and hospital stays and broaden treatment options appropriate to idiopathic TTP if the variant is unrecognisable to wildtype ADAMTS13 auto-antibodies.

Subarachnoid haemorrhage (SAH) accounts for 5% of all strokes and is associated with very poor outcomes with fatality rates in 35% in the first three months and only ˜55% of patients regaining independent function (Macdonald and Schweizer 2017). Following the early phase of brain injury one third of patients experience delayed cerebral ischemia caused by microthrombosis 3-14 days after haemorrhage leading to further inflammation and neurological deterioration. SAH patient studies reveal elevated VWF and lowered ADAMTS13 levels following bleeding compared to healthy controls (Kumar et al. 2017). Protection against microglial activation and neuronal injury is observed in VWF deficient mice post-SAH (Wan et al. 2018) and systemic administration of ADAMTS13 ameliorated microthrombosis, apoptosis and neuroinflammation, Improving neurological function (Muroi et al. 2014). Evidence also suggests a role of the ADAMTS13-VWF axis in intracerebral haemorrhage (ICH). Recombinant ADAMTS13 was shown to decrease microglial activation, neutrophil accumulation, protect against blood brain barrier breakdown and improve neurological outcomes in a murine ICH model (Cai et al. 2015).

Chronic Thromboembolic Pulmonary Hypotension (CTEPH) Is a progressive disease caused by disseminated thrombi in the pulmonary vasculature, which if left untreated can lead to right-sided heart failure. Newnham and colleagues demonstrated CTEPH patients have decreased ADAMTS13 and increased VWF plasma levels compared to healthy controls (Newnham et al. 2019). Currently, removal of pulmonary obstructions by surgery is the definitive treatment option for CTEPH however the potential thrombolytic use of ADAMTS13 could circumvent problems which exist with operability.

The importance of VWF-ADAMTS13 in murine models of Myocardial Infarction (MI) has been clearly demonstrated (Witsch et al. 2018). A meta-analysis of patient data described low levels of ADAMTS13 associated with increased risk of MI, however, the study did not find a synergy with high VWF levels conflicting with the relationship seen in ischemic stroke (Maino et al. 2015). In ST-elevation myocardial infarction (STEMI) patients, MI is associated with higher VWF and lower ADAMTS13 activity, however administration of recombinant ADAMTS13 in a porcine model of acute MI showed no effect on infarct size (Eerenberg et al. 2016). In unstable angina (UA) patients, an unfavourable VWF:ADAMTS13 ratio remained for up to 6 months after the acute phase, indicating a prolonged period of thrombogenicity and increased potential of platelet clot formation In the chronic phase (Fuchigami et al. 2008).

ADAMTS13 deficiency has also been linked to other ischemia/reperfusion pathologies. Using data from the Rotterdam Study, a general population based study In the Netherlands, Sedaghat et al found that higher VWF ADAMTS13 ratio and lower ADAMTS13 levels are independently associated with a steeper decline in kidney function (Sedaghat et al. 2016). More recently, using murine models of kidney ischemia/reperfusion injury two parallel studies demonstrated the protective action of recombinant ADAMTS13 in alleviating immune cell infiltration and kidney damage (Zhou et al. 2019; Ono et al. 2019). These results are corroborated in VWF−/− mice which sustain less kidney damage than wildtype controls (Ono et al. 2019). In addition, Urisono et al., report on partial protection against hepatic ischemia/reperfusion injury in VWF−/− mice compared to wildtype controls (Urisono et al. 2018).

In diabetic patients, clinical reports suggest an association between low levels of ADAMTS13 and microvascular complications such as nephropathy (kidney disease) (Taniguchi et al. 2010). Interestingly, one study demonstrated an ADAMTS13 single nucleotide polymorphism [Pro618Ala] reduced the proteolytic activity of ADAMTS13 and was associated with increased incidence of renal and cardiovascular events in type 2 diabetic patients (Rurali et al. 2013). Animal models show decreased kidney function is exacerbated by ADAMTS13 deficiency In wildtype mice and ameliorated In ADAMTS13−/− VWF−/− double knockout mice, displaying a VWF dependent effect (Dhanesha et al. 2017). These results indicate the VWF-ADAMTS13 axis is responsible at least in part for the endothelial dysfunction which occurs in diabetic vascular pathology and that patients could benefit from ADAMTS13 therapy.

Almost 30 years ago the BMJ reported that patients with Crohn's Disease, ulcerative colitis and bacterial diarrhoea have higher levels of circulating VWF (Stevens et al. 1992). More recently, a murine model of colitis demonstrated enhanced VWF-rich thrombi in the colon and propagation of intestinal inflammation in ADAMTS13 deficient mice which was reduced upon administration of recombinant human ADAMTS13. The same paper also identified areas of marked VWF staining in human colitis colonic tissue compared to healthy controls, providing a strong case for VWF-ADAMTS13 imbalance in exacerbation of inflammatory bowel disease (Zitomersky et al. 2017). The proposed mechanism of increased VWF release from the colonic endothelium, thrombocytosis, ischemia and further VWF release presents an opportunity for an ADAMTS13 intervention aimed at breaking this vicious cycle and limiting further detrimental inflammation. It is likely that there are still many undocumented indications for therapeutic use of ADAMTS13 given that much of the research discussed here has been conducted only in the last 5 years. Analysis of population data, such as that provided by The Rotterdam Study, can help to pinpoint certain diseases where abnormal VWF or ADAMTS13 activity might play a role in pathology. Indeed analysis of data collected in the same study highlighted a link between low ADAMTS13 levels and risk of dementia (Wolters et al. 2018). As research continues to identify pathological inflammation as an instigator in a wide variety of diseases, applications such as this one could prove to be invaluable in multiple settings.

In some embodiments, the disease/condition to be treated/prevented in accordance with the present disclosure is characterised by an increased level and/or activity of VWF, and/or an increased level and/or activity of a complex comprising VWF (e.g. as compared to the level in the healthy state (i.e. in the absence of the disease/condition)). In some embodiments, the disease/condition to be treated/prevented is characterised by a reduced level of ADAMTS13 and/or a reduced level of ADAMTS13 proteolytic activity (e.g. as compared to the level in the healthy state (i.e. in the absence of the disease/condition)). A reduced level of ADAMTS13 and/or a reduced level of ADAMTS13 proteolytic activity may be referred to herein as ‘ADAMTS13 insufficiency’.

In some embodiments, the articles of the present disclosure are provided for use in the treatment/prevention of thrombosis, or a disease/condition characterised by thrombosis. The articles of the present disclosure may be employed as anti-clotting/anti-coagulant agents.

In some embodiments, the articles of the present disclosure are provided for use in the treatment/prevention of inflammation, or a disease/condition characterised by inflammation.

In some embodiments, the disease/condition to be treated/prevented in accordance with the present disclosure selected from: a disease/condition characterised by thrombosis, a disease/condition characterised by inflammation, thrombotic thrombocytopenic purpura (TTP), ischaemic stroke, haemorrhagic stroke, subarachnoid haemorrhage (SAH), intracerebral haemorrhage (ICH), chronic thromboembolic pulmonary hypotension (CTEPH), myocardial infarction (MI), ST-elevation myocardial infarction (STEMI), unstable angina (UA), ischemia, reperfusion, deep venous thrombosis, pulmonary embolism, intravascular coagulation (DIC), hemolytic-uremic syndrome (HUS), cerebral infarction, systemic lupus erythematosus (SLE), disease cause by infection with a SARSr-CoV (e.g. SARS-CoV-2; e.g. COVID-19), acute respiratory distress syndrome (ARDS), pneumonia, kidney damage, nephropathy, microvascular diseases, dementia, Crohn's disease, inflammatory bowel disease, ulcerative colitis, and bacterial diarrhoea.

Administration of the articles of the present disclosure is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration may be alone or in combination with other treatments, either simultaneously or sequentially dependent upon the condition to be treated. The articles of the present disclosure and a therapeutic/prophylactic agent may be administered simultaneously or sequentially.

Simultaneous administration refers to administration of an ADAMTS13 variant, nucleic acid, vector, cell or composition of the disclosure and another therapeutic/prophylactic agent together, for example as a pharmaceutical composition containing both agents (combined preparation), or immediately after each other and optionally via the same route of administration, e.g. to the same artery, vein or other blood vessel. Sequential administration refers to administration of one of: (i) an ADAMTS13 variant, nucleic acid, vector, cell or composition of the disclosure and (ii) another therapeutic/prophylactic agent, followed after a given time interval by separate administration of the other agent. It is not required that the two agents are administered by the same route, although this is the case in some embodiments. The time interval may be any time interval.

Multiple doses of an ADAMTS13 variant, nucleic acid, vector, cell or composition of the disclosure may be provided. One or more, or each, of the doses may be accompanied by simultaneous or sequential administration of another therapeutic/prophylactic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

### Subjects

The subject in accordance with aspects of the present disclosure may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer), may be suspected of having such a disease/condition, or may be at risk of developing/contracting such a disease/condition.

In embodiments according to the present disclosure the subject is preferably a human subject. In some embodiments, the subject to be treated according to a therapeutic or prophylactic method of the present disclosure is a subject having, or at risk of developing, a disease described herein. In embodiments according to the present disclosure, a subject may be selected for treatment according to the methods based on characterisation for certain markers of such disease/condition.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof. While the disclosure has been described in conjunction with the exemplary embodiments described above, many equivalent modifications and variations will be apparent to those skilled in the art when given this disclosure. Accordingly, the exemplary embodiments of the disclosure set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “Include”, and variations such as “comprises”, “comprising”, and “Including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” Include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Where a nucleic acid sequence is disclosed herein, the reverse complement thereof is also expressly contemplated.

Methods described herein may preferably be performed in vitro. The term “in vitro” is intended to encompass procedures performed with cells in culture whereas the term “in vivo” is intended to encompass procedures with/on intact multi-cellular organisms.

### EXAMPLES

### Example 1—Materials and Methods

### Protein Expression and Purification

A pCDNA3.1 28 construct encoding recombinant human ADAMTS13 with a C-terminal Myc/His6 tag was used to generate variants by site-directed mutagenesis.

Constructs were generated encoding ADAMTS13 variants comprising the following substitutions in the linker 3 region of the ADAMTS13 protein: A1144V, A1145V, A1146V, P1147V, P1154V, P1171V, P1173V, P1175V, P1180V, and P1182V. A construct encoding the known ADAMTS13 R568K/F592Y/R660K/Y661F/Y665F variant was also produced.

The amino acid sequences of the Myc/His6-tagged human ADAMTS13 and ADAMTS13 variants characterised in the present Examples are shown in SEQ ID NOs:72 to 83, and the nucleotide sequences encoding the recombinant polypeptides are shown in SEQ ID NOs:84 to 95.

Myc/His6-tagged human ADAMTS13 and ADAMTS13 variants were expressed from CHO-K1 cell lines stably expressing constructs encoding the proteins, and subsequently purified by fast protein liquid chromatography (FPLC) with zinc-coupled HiTrap chelating columns (GE Healthcare, Chicago, IL, USA). CHO-expressed ADAMTS13, for use in the murine stroke model, was passed over a hydroxyapatite column to remove contaminating proteins, and the purified ADAMTS13 protein was quantified by ELISA and dialyzed into 150 mm NaCl, 20 mm histidine, 2% sucrose, and 0.05% Tween-80 (pH 7.4).

Corresponding constructs were also generated encoding ADAMTS13 variants comprising the following substitutions in the linker 3 region of the ADAMTS13 protein: A1144K, A1144I, A1145K, A1145I, A1146K, A1146I, P1147K, P1147I, P1154K, P1154I, P1171K, P1171I, P1173K, P1173I, P1175K, P1175I, P1180K, P1180I, P1182K, and P1182I. The amino acid sequences of these ADAMTS13 variants are shown in SEQ ID NOs:96 to 115, and the nucleotide sequences encoding the recombinant polypeptides are shown in SEQ ID NOs:116 to 135. These ADAMTS13 variants were transiently expressed in HEK293S cells and harvested in concentrated conditioned media. Protein levels were quantified by in-house ADAMTS13 ELISA and corroborated by western blot.

### FRETS-VWF73 Assay

FRETS-VWF73 assays of ADAMTS13-mediated proteolysis of VWF were performed as described in South et al., 2018.

Briefly, purified Myc/His6-tagged human ADAMTS13 and ADAMTS13 variants were diluted to a concentration of 0.3 nM in 5 mM Bis-Tris pH 6.0, 25 mM CaCl₂, and 0.005% Tween-20, in white 96-well plates (Nunc).

In some experiments, purified VWF D4-CK fragment may be added to a final concentration of 20-60 nM, followed by a 45 min preincubation at 37° C. In other experiments, VWF D4-CK fragment was not added.

Proteolysis was subsequently initiated by the addition of an equal volume of 4 μM FRETS-VWF73 substrate (Peptanova). Fluorescence (excitation, 340 nm; emission, 460 nm) was measured at 30° C. at 1 min intervals for 1 h using a Fluostar Omega plate reader (BMG Labtech). Fluorescence measurements were normalised to values obtained for wildtype human ADAMTS13.

Vena8 Fluoro+ biochips (Cellix) were coated with 200 μg/ml collagen type III (Southern Biotech) and blocked with 1% BSA, 1 mg/ml glucose in HEPES buffer. Washed platelets combined with red blood cells were treated with 100 nM PGE1 and 75 mU/ml Apyrase, to prevent platelet activation, before platelets were labelled with 10 μM DiOC₆. Platelets were supplemented with 10 μg/ml multimeric plasma VWF and perfused over the collagen surface at a constant shear rate of 1500 s-1 (at which platelet capture is VWF dependent) for 5 minutes. Adhesion of labelled platelets was visualised by fluorescence imaging at 250 ms intervals using a 20× objective and analysed using Slidebook software to determine platelet coverage (%) at 270 seconds. To determine the effect of ADAMTS13 on platelet capture the assay was also performed in the presence of WT or variant ADAMTS13 at a range of concentrations and EC₅₀ values were determined by dose-response curves.

### Murine Focal Ischemia Stroke Model

ADAMTS13 was administered as a bolus by tail vein catheter 1 hour after MCA occlusion. Dosage was 4 μl/g of a 1.5 mg/ml preparation to give a final dose of 6 mg/kg. FeCl3-induced occlusion of the middle cerebral artery (MCA) was performed using surgical techniques described previously (Denorme et al., 2016). Regional cerebral blood flow (rCBF) In the MCA territory was determined by Laser Speckle contrast imaging, and infarct size was determined 24 h after occlusion of the MCA by staining brain sections (10 μm PFA fixed cryosections) with cresyl violet. Brain sections were also stained with haematoxylin and eosin and by immunofluorescence using antibodies against VWF (Dako, rabbit polyclonal A0082) and fibrinogen (Thermo-Fisher, sheep polyclonal PA1-85429).

### Treatment of Respiratory Tract Infection

Mice were infected with a mildly virulent strain of Streptococcus pneumoniae (ascending inoculum over days 1-6), and administered on day 8 with human ADAMTS13, an ADAMTS13 variant or vehicle (as a negative control). Inflammation of the cerebral vasculature was determined at day 18 using gradient-echo MRI and a VWF-specific MPIO contrast agent.

ADAMTS13 was administered as a bolus by tail vein catheter on Day 8. Dosage was 4 μl/g of a 1.5 mg/ml preparation to give a final dose of 6 mg/kg. Amoxicillin, when used, was also administered on day 8 by a single subcutaneous injection at a final dosage of 400 mg/kg. Efficacy of caADAMTS13 in this model was defined as a reduction in R2* in MGE MRI scans (indicating reduced vascular inflammation in the brain) and/or reduced reactive VWF species in the plasma.

### FeCl3-Induced Occlusion of the Middle Cerebral Artery (MCA)

Mice are treated using surgical techniques described previously (Denorme et al., 2016) to induce occlusion of the MCA. Vehicle or caADAMTS13 (6 mg/kg) is administered as a bolus by tail vein catheter 4 hours after MCA occlusion. Regional cerebral blood flow (rCBF) in the MCA territory is determined by Laser Speckle contrast imaging for 1 hour following ADAMTS13 administration, and infarct size is determined 24 h after occlusion of the MCA by staining brain sections (10 μm PFA fixed cryosections) with cresyl violet. Brain sections are stained with haematoxylin and eosin and by immunofluorescence using antibodies against VWF (Dako, rabbit polyclonal A0082) and fibrinogen (Thermo-Fisher, sheep polyclonal PA1-85429).

### Example 2—Results

### In Vitro Activity

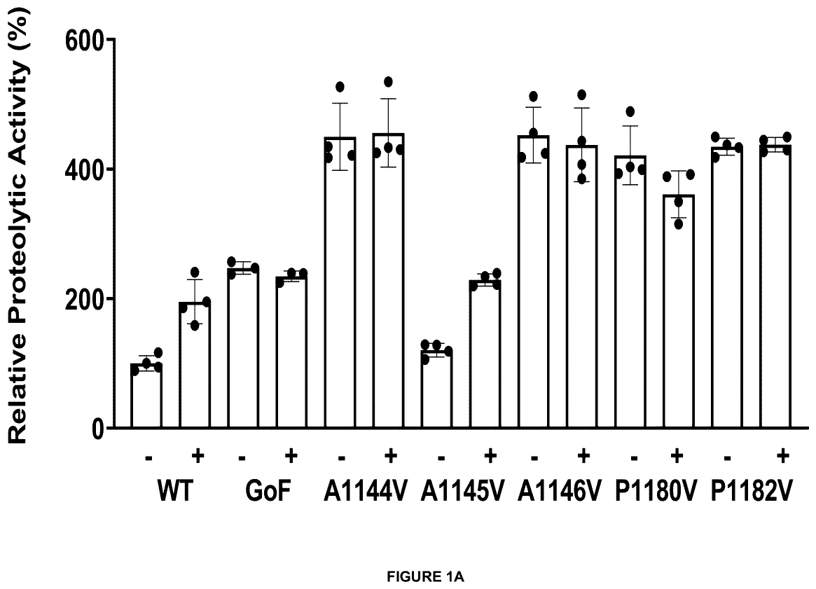

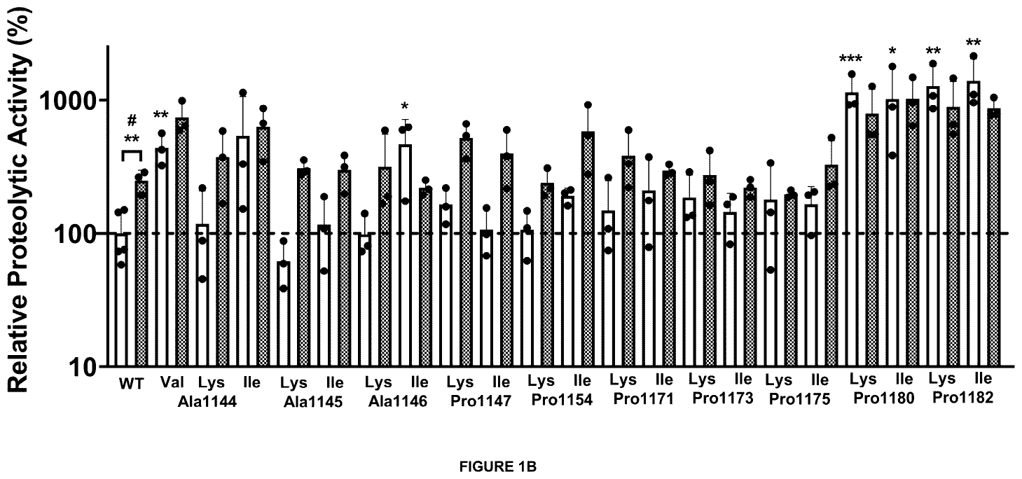

The proteolytic activity of wild type ADAMTS13, the known GoF ADAMTS13 R568K/F592Y/R660K/Y661F/Y665F variant and linker 3 variants was evaluated by FRETS-VWF73 assay in both the absence (−) and presence (+) of the activating VWF-D4CK domain fragment (FIG. 1A).

The residues targeted In the ADAMTS13 variants were selected on the basis of being those that are most likely to significantly influence the secondary structure and thereby, the activity of the protein. Specifically, the cluster of alanine residues (Ala1144Val, Ala1145Val and Ala1146Val) were selected on the basis that these residues may provide flexibility to the protein structure in this region. Subsequent proline residues (Pro1147Val, Pro1154Val, Pro1171Val, Pro1173Val, Pro1175Val, Pro1180Val, and Pro1182Val) were selected on the basis that these residues are likely to constrain and thereby strongly influence the secondary structure of the protein.

Indeed, the nature of the amino acid residues that were selected for introduction into the ADAMTS13 variants were not identified based on not the typical approach of chemical similarity (e.g. degree of charge and structure conservation), but based on the degree of flexibility provided by these residues. Specifically, in comparison to alanine, residues including, for example, lysine, valine, and isoleucine, have been found to significantly reduce flexibility, to an extent approaching that of proline.

A1144V, A11146V, P1180V, and P1182V displayed significantly higher proteolytic activity compared to wildtype ADAMTS13 and the R568K/F592Y/R660K/Y661F/Y665F variant (FIG. 1A). This significant difference was observed both in the absence (−) and presence (+) of the activating VWF-D4CK domain fragment. In the absence of the activating VWF-D4CK domain fragment, the proteolytic activity of A1144V, A1146V, P1180V, and P1182V variants was more than 4-fold higher than wildtype ADAMTS13.

Substitution of alanine at positions 1144 and 1146 with isoleucine, an amino acid with a similar rigidity to valine, induces a similar constitutive activity to that seen with the A1144V, A1146V variants (FIG. 1B). The replacement of valine with either lysine or isoleucine at positions 1180 and 1182 Induces greater than 10-fold increase in activity. This is higher than the P1180V and P1182V variants and higher than A1144V.

VWF-mediated capture of platelets under arterial shear stress was measured for wildtype ADAMTS13, the R568K/F592Y/R660K/Y661F/Y665F variant, and the A1144V variant. The results show that the A1144V variant was more effective at reducing platelet coverage than wildtype ADAMTS13 or the R568K/F592Y/R660K/Y661F/Y665F variant. The A1144V variant was able to reduce platelet coverage to a level comparable to the negative control condition at a concentration of 2.5 nM. The A1144V variant was found to be much more potent than wildtype ADAMTS13 or the R568K/F592Y/R660K/Y661F/Y665F variant at reducing platelet coverage in this assay.

### Results of Laser Speckle Contrast Analysis

Laser speckle contrast imaging provides a quantitative map of regional cerebral blood flow in the mouse brain. Flux values are derived from a region of interest (corresponding to the tissue supplied by the middle cerebral artery) in the ipsilateral (stroked) hemisphere and an identical control region in the contralateral hemisphere. A decrease in the ratio of these flux values indicates a drop in blood flow in the stroked hemisphere compared to the control region. Typically, the flux ratio is reduced to approximately 0.4 in an animal with MCAo compared to a value of 1 in sham animals. An increase in flux ratio over time, observed in animals treated with caADAMTS13, Indicates that the occlusive thrombus has been cleared and blood flow restored to the MCA territory.

### Lesion Volumes

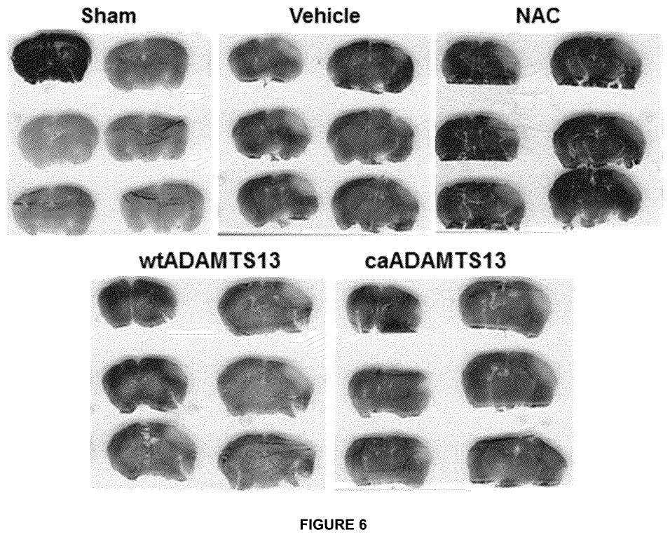

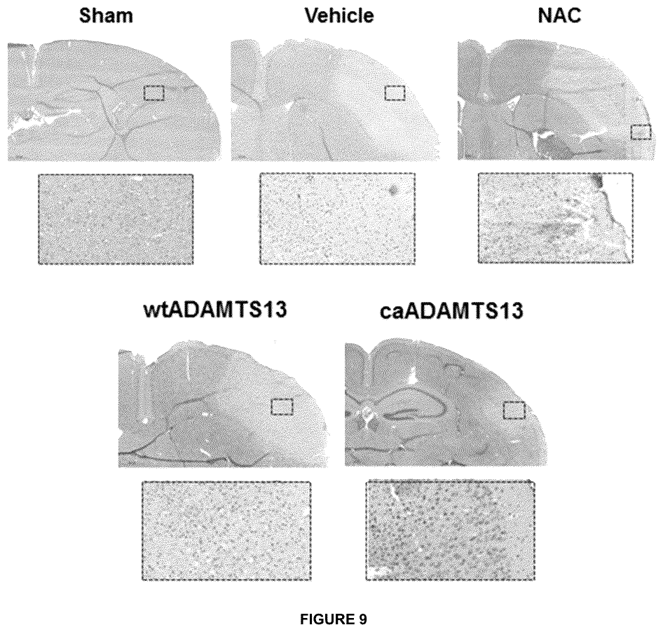

FIGS. 6 to 9 show the effect of treatment with wildtype ADAMTS13 or the A1144V variant on lesion volumes in the ischemia stroke model, at 24 hours post-occlusion.

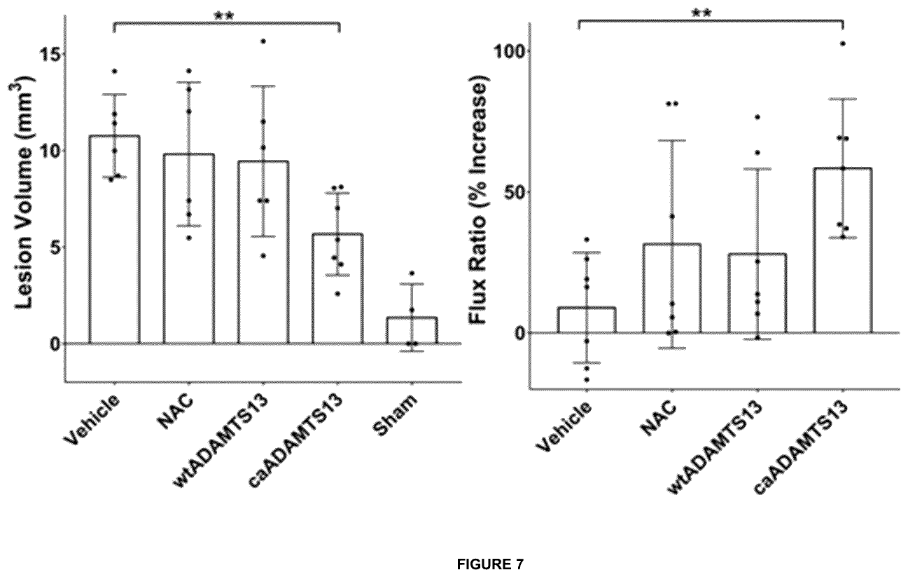

FIG. 7 shows that treatment with the A1144V variant (referred to in FIG. 7 as ‘caADAMTS13’) reduced lesion volume as compared to treatment with the vehicle control, N-acetyl-cysteine (NAC), or wildtype ADAMTS13 (wtADAMTS13).

Treatment with the A1144V variant was also found to result in a greater percentage increase the flux ratio between the ipsilateral and contralateral ROIs between 0 and 60 minutes after injection, than treatment with the vehicle control, N-acetyl-cysteine (NAC), or wildtype ADAMTS13 (wtADAMTS13) (FIG. 7).

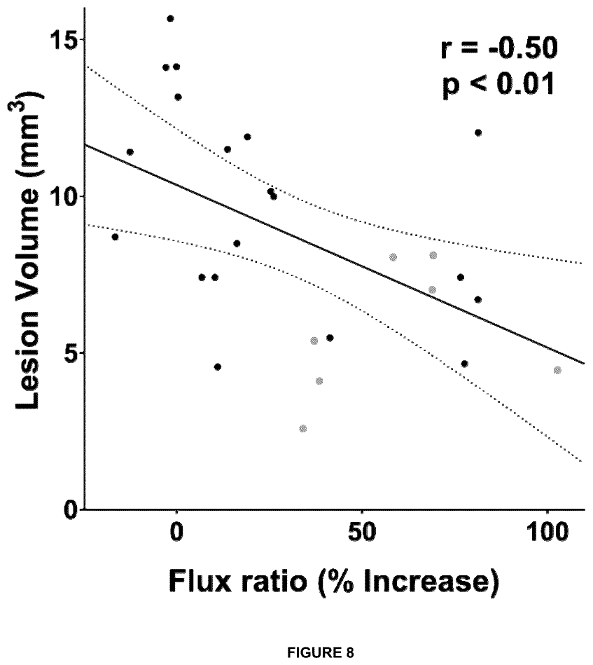

A significant negative correlation was observed between the increase in flux ratio and lesion volume, across all treatment groups (FIG. 8).

An increase in flux ratio over time indicates that the occlusive thrombus has been cleared and blood flow restored to the MCA territory, thereby salvaging viable tissue as indicated by the correlating reduction in lesion volume.

### Residual Thrombus Content

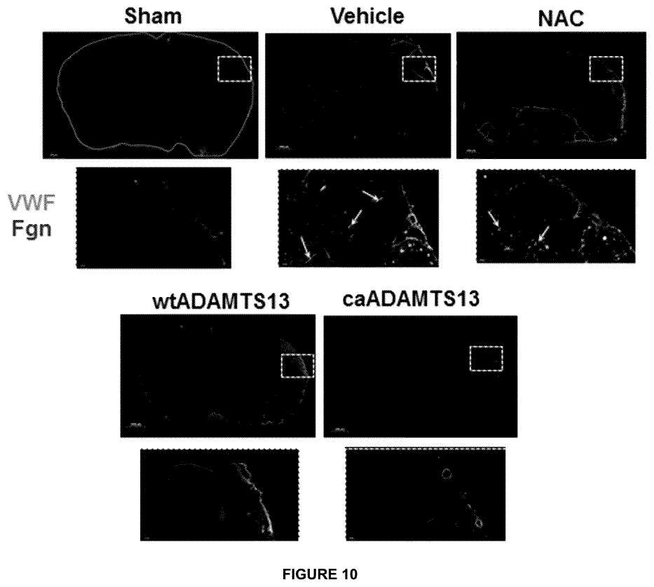

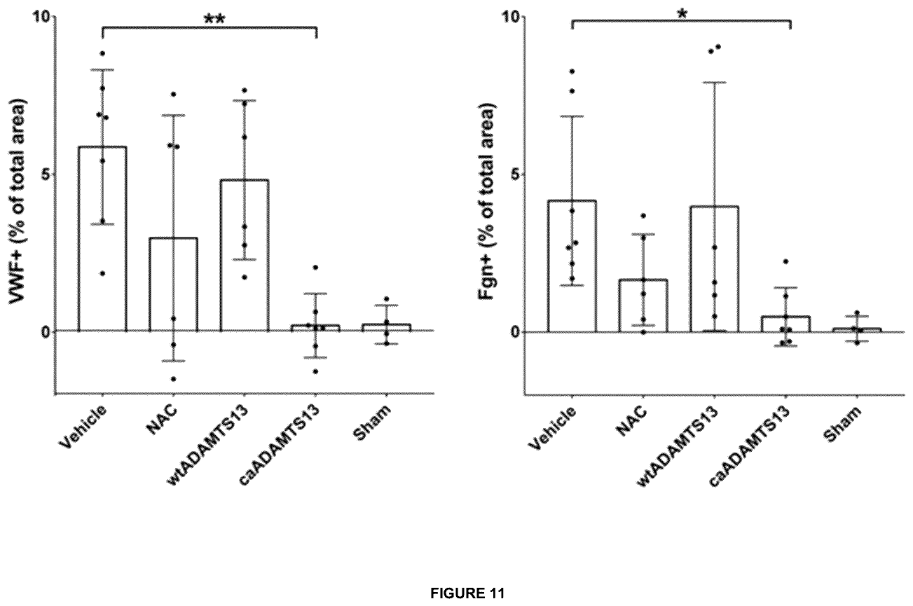

FIGS. 10 and 11 show that VWF and fibrin(ogen) content at site of FeCl3 application is lower in brain sections obtained from mice treated with the A1144V variant (referred to as ‘caADAMTS13’) than treatment with the vehicle control, N-acetyl-cysteine (NAC), or wildtype ADAMTS13 (wtADAMTS13).

Thrombi are composed primarily of a combination of fibrin and VWF-platelet aggregates. Thrombus composition in humans can vary, with differing contributions of these two components, so that thrombolytics acting only on fibrin (rt-PA) or only on VWF (NAC, WT ADAMTS13) may not fully dissolve the occlusive thrombi. The reduction in both VWF and fibrin observed here, supports in vitro data indicating that caADAMTS13 is capable of proteolysing both VWF and fibrinogen thereby circumventing the issue of thrombus composition.

### Treatment of Respiratory Tract Infection

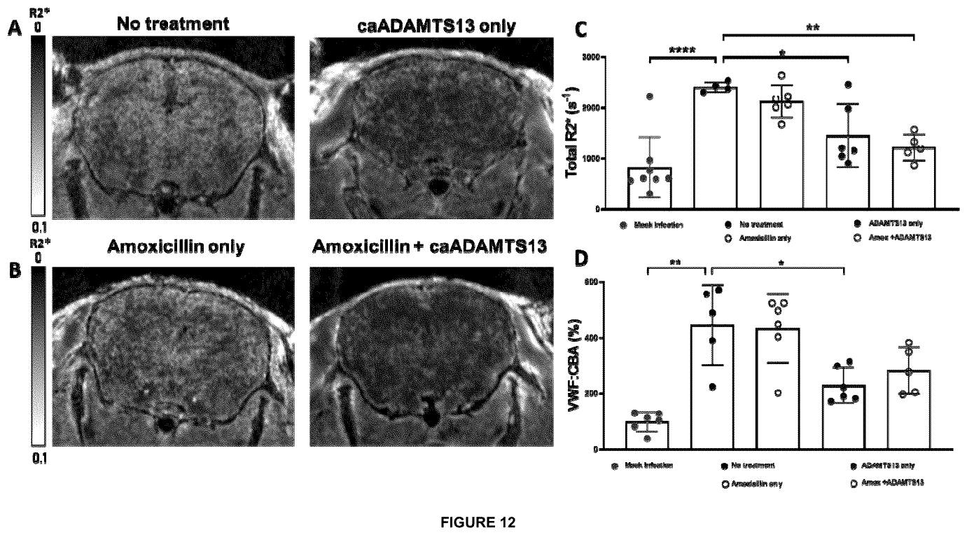

FIG. 12 shows the effect of treatment with wildtype ADAMTS13 or the A1144V variant on cerebral inflammation in mice infected with Streptococcus pneumoniae.

Animals treated with the A1144V variant (caADAMTS13)—either alone or in combination with amoxicillin—displayed a ˜50% reduction in cerebral inflammation.

In response to localised respiratory tract infection (RTI) there is release of large VWF multimers from the vasculature of the lung which circulate in the plasma. The infection also causes an increase in the plasma concentration of pro-inflammatory cytokines (IL-6, IL-1 etc) which can initiate vascular inflammation in other organs including the brain (FIGS. 12A and C). These changes are believed to be a major contributor to the increased risk of stroke observed in patents with preceding RTI which may account for as many as 10% of all strokes.

This response continues long after the infection is resolved by antibiotics with both plasma VWF and cerebral VWF levels remaining high (FIGS. 12B, C and D). By dampening this response, caADAMTS13 may represent a potential prophylactic option for the prevention of strokes triggered by RTI.

### FeCl3-Induced Occlusion of the Middle Cerebral Artery (MCA)

Treatment with caADAMTS13 elicits an increase in flux ratio over time (compared to vehicle control) indicating that the occlusive thrombus has been cleared and blood flow restored to the MCA territory, thereby salvaging viable tissue as indicated by a correlating reduction in lesion volume. Delayed administration does not result in haemorrhagic transformation as is the case for rT-PA.

### REFERENCES

A number of publications are cited above in order to more fully describe and disclose the invention and the state of the art to which the disclosure pertains. Full citations for these references are provided below. The entirety of each of these references is incorporated herein.
1. Bhatia, R., M. D. Hill, N. Shobha, B. Menon, S. Bal, P. Kochar, T. Watson, M. Goyal, and A. M. Demchuk. 2010. ‘Low rates of acute recanalization with intravenous recombinant tissue plasminogen activator in ischemic stroke: real-world experience and a call for action’, Stroke, 41: 2254-8.
2. Cal, P., H. Luo, H. Xu, X. Zhu, W. Xu, Y. Dal, J. Xiao, Y. Cao, Y. Zhao, B. Q. Zhao, and W. Fan. 2015. ‘Recombinant ADAMTS 13 Attenuates Brain Injury After Intracerebral Hemorrhage’, Stroke, 46:2647-53.
3. Chauhan, A. K., J. Kisucka, A. Brill, M. T. Walsh, F. Scheiflinger, and D. D. Wagner. 2008. ‘ADAMTS13: a new link between thrombosis and inflammation’, J Exp Med, 205: 2065-74.
4. L. Deforche, E. Roose, A. Vandenbulcke, N. Vandeputte, H. B. Feyst, T. A. Springer‡, L. Z. Mi, J. Muia§, J. E. Sadler§, K. Soejima, H. Rottensteiner, H. Deckmyn, S. F. De Meyer and K. Vanhoorelbeke. 2915. ‘Linker regions and flexibility around the metalloprotease domain account for conformational activation of ADAMTS13’ J Thromb Haemost. 13(11): 2063-2075. doi:10.111.1/jth.13149.
5. Denorme, F., F. Langhauser, L. Desender, A. Vandenbulcke, H. Rottensteiner, B. Plaimauer, O. Francois, T. Andersson, H. Deckmyn, F. Scheiflinger, C. Kleinschnitz, K. Vanhoorebeke, and S. F. De Meyer. 2016. ‘ADAMTS13-mediated thrombolysis of t-PA-resistant occlusions in ischemic stroke in mice’, Blood, 127: 2337-45.
6. De Meyer S F, Stoll G, Wagner D D, Kleinschnitz C. von Willebrand factor: an emerging target in stroke therapy. Stroke 2012; 43: 599-606.
7. De Meyer S F, Savchenko A S, Haas M S, Schatzberg D, Carroll M C, Schiviz A, Dietrich B, Rottensteiner H, Scheiflinger F, Wagner D D. Protective anti-inflammatory effect of ADAMTS13 on myocardial ischemia/reperfusion injury in mice. Blood 2012; 120: 5217-23.
8. Dhanesha, N., P. Doddapattar, M. R. Chorawala, M. K. Nayak, K. Kokame, J. M. Staber, S. R. Lentz, and A. K. Chauhan. 2017. ‘ADAMTS13 Retards Progression of Diabetic Nephropathy by Inhibiting Intrarenal Thrombosis In Mice’, Arterioscler Thromb Vasc Biol, 37:1332-38.
9. Eerenberg, E. S., P. F. Teunissen, B. J. van den Born, J. C. Meijers, M. R. Hollander, M. Jansen, R. Tijssen, J. A. Belien, P. M. van de Von, M. F. Aly, O. Kamp, H. W. Niessen, P. W. Kamphuisen, M. Levi, and N. van Royen. 2016. ‘The role of ADAMTS13 in acute myocardial infarction: cause or consequence?’, Cardiovasc Res, 111: 194-203.
10. Emberson, J., K. R. Lees, P. Lyden, L. Blackwell, G. Albers, E. Bluhmki, T. Brott, G. Cohen, S. Davis, G. Donnan, J. Grotta, G. Howard, M. Kaste, M. Koga, R. von Kummer, M. Lansberg, R. I. Lindley, G. Murray, J. M. Olivot, M. Parsons, B. Tilley, D. Toni, K. Toyoda, N. Wahigren, J. Wardlaw, W. Whiteley, G. J. del Zoppo, C. Baigent, P. Sandercock, W. Hacke, and Group Stroke Thrombolysis Trialists' Collaborative. 2014. ‘Effect of treatment delay, age, and stroke severity on the effects of intravenous thrombolysis with alteplase for acute ischaemic stroke: a meta-analysis of individual patient data from randomised trials’, Lancet, 384: 1929-35.
11. Fuchigami, S., K. Kaikita, K. Soejima, M. Matsukawa, T. Honda, K. Tsujita, Y. Nagayoshi, S. Kojima, T. Nakagaki, S. Sugiyama, and H. Ogawa. 2008. ‘Changes in plasma von Willebrand factor-cleaving protease (ADAMTS13) levels in patients with unstable angina’, Thromb Res, 122: 618-23.
12. Fujioka, M., K. Hayakawa, K. Mishima, A. Kunizawa, K. Irie, S. Higuchi, T. Nakano, C. Muroi, H. Fukushima, M. Sugimoto, F. Banno, K. Kokame, T. Miyata, M. Fujiwara, K. Okuchi, and K. Nishio. 2010. ‘ADAMTS13 gene deletion aggravates ischemic brain damage: a possible neuroprotective role of ADAMTS13 by ameliorating postischemic hypoperfusion’, Blood, 115: 1650-3.
13. Jian, C., Xiao, J., Gong, L., Skipwith, C. G., Jin, S. Y., Kwaan, H. C., and Zheng, X. L. (2012) Gain-of-function ADAMTS13 variants that are resistant to autoantibodies against ADAMTS13 in patients with acquired thrombotic thrombocytopenic purpura. Blood 119, 3836-3843.
14. Khan, M. M., D. G. Motto, S. R. Lentz, and A. K. Chauhan. 2012. ‘ADAMTS13 reduces VWF-mediated acute Inflammation following focal cerebral ischemia in mice’, J Thromb Haemost, 10: 1665-71.
15. Kleinschnitz, C., S. F. De Meyer, T. Schwarz, M. Austinat, K. Vanhoorelbeke, B. Nieswandt, H. Deckmyn, and G. Stoll. 2009. ‘Deficiency of von Willebrand factor protects mice from ischemic stroke’, Blood, 113: 3600-3.
16. Kolaczkowska, E., C. N. Jenne, B. G. Surewaard, A. Thanabalasuriar, W. Y. Lee, M. J. Sanz, K. Mowen, G. Opdenakker, and P. Kubes. 2015. ‘Molecular mechanisms of NET formation and degradation revealed by intravital imaging in the liver vasculature’, Nat Commun, 6: 6673.
17. Kremer Hovinga, J. A., P. Coppo, B. Lammle, J. L. Moake, T. Miyata, and K. Vanhoorelbeke. 2017. ‘Thrombotic thrombocytopenic purpura’, Nat Rev Dis Primers, 3:17020.
18. Kumar, M., W. Cao, J. K. McDaniel, H. P. Pham, D. Raju, K. Nawalinski, S. Frangos, D. Kung, E. Zager, S. E. Kasner, J. M. Levine, and X. L. Zheng. 2017. ‘Plasma ADAMTS13 activity and von Willebrand factor antigen and activity In patients with subarachnold haemorrhage’, Thromb Haemost, 117: 691-99.
19. Macdonald, R. L., and T. A. Schweizer. 2017. ‘Spontaneous subarachnoid haemorrhage’, Lancet, 389: 655-66.
20. Maino, A., B. Siegerink, L. A. Lotta, J. T. Crawley, S. le Cessie, F. W. Leebeek, D. A. Lane, G. D. Lowe, F. Peyvandi, and F. R. Rosendaal. 2015. ‘Plasma ADAMTS-13 levels and the risk of myocardial infarction: an individual patient data meta-analysis’, J Thromb Haemost, 13:1396-404.
21. Martinod, K., and D. D. Wagner. 2014. ‘Thrombosis: tangled up in NETs’, Blood, 123: 2768-76.
22. Moake J L, Rudy C K, Troll J H, Weinstein M J, Colannino N M, Azocar J, Seder R H, Hong S L, Deykin D (1982) N Engl J Med 307:1432-1435, pmid:6813740.
23. Muroi, C., M. Fujioka, K. Mishima, K. Ire, Y. Fujimura, T. Nakano, J. Fandino, E. Keller, K. Iwasaki, and M. Fujiwara. 2014. ‘Effect of ADAMTS-13 on cerebrovascular microthrombosis and neuronal injury after experimental subarachnoid hemorrhage’, J Thromb Haemost, 12: 505-14.
24. Nakano, T., K. Irie, K. Hayakawa, K. Sano, Y. Nakamura, M. Tanaka, Y. Yamashita, T. Satho, M. Fujioka, C. Muroi, K. Matsuo, H. Ishikura, K. Futagami, and K. Mishima. 2015. ‘Delayed treatment with ADAMTS13 ameliorates cerebral ischemic injury without hemorrhagic complication’, Brain Res, 1624: 330-35.
25. National Institute of Neurological, Disorders, and P. A. Stroke Study Group Stroke rt. 1995. ‘Tissue plasminogen activator for acute ischemic stroke’, N Engl J Med, 333:1581-7.
26. Newnham, M., K. South, M. Bleda, W. R. Auger, J. A. Barbera, H. Bogaard, K. Bunclark, J. E. Cannon, M. Delcroix, C. Hadinnapola, L. S. Howard, D. Jenkins, E. Mayer, C. Ng, C. J. Rhodes, N. Screaton, K. Sheares, M. A. Simpson, M. Southwood, L. Su, D. Taboada, M. Traylor, R. C. Trembath, S. S. Villar, M. R. Wilkins, J. Wharton, S. Graf, J. Pepke-Zaba, M. Laffan, D. A. Lane, N. W. Morrell, and M. Toshner. 2019. ‘The ADAMTS13-VWF axis is dysregulated in chronic thromboembolic pulmonary hypertension’, Eur Respir J, 53.
27. Ono, S., H. Matsui, M. Noda, S. Kasuda, N. Yada, K. Yoshimoto, M. Akiyama, T. Miyata, M. Sugimoto, and K. Nishio. 2019. ‘Functional regulation of von Willebrand factor ameliorates acute ischemia-reperfusion kidney injury in mice’, Sci Rep, 9: 14453.
28. Rurali, E., M. Noris, A. Chianca, R. Donadelli, F. Banterla, M. Galbusera, G. Gherardi, S. Gastoldi, A. Parvanova, I. Iliev, A. Bossi, C. Haefliger, R. Trevisan, G. Remuzzi, P. Ruggenenti, and Benedict Study Group. 2013. ‘ADAMTS13 predicts renal and cardiovascular events in type 2 diabetic patients and response to therapy’, Diabetes, 62: 3599-609.
29. Sadler, J. E. 2008. ‘Von Willebrand factor, ADAMTS13, and thrombotic thrombocytopenic purpura’, Blood, 112:11-8.
30. Sadler J. E. von Willebrand factor assembly and secretion. J Thromb Haemost 2009; 7(Suppl. 1): 24-7.
31. Scully, M., P. Knobl, K. Kentouche, L. Rice, J. Windyga, R. Schneppenheim, J. A. Kremer Hovinga, M. Kajiwara, Y. Fujimura, C. Maggiore, J. Doralt, C. Hibbard, L. Martell, and B. Ewenstein. 2017. ‘Recombinant ADAMTS-13: first-in-human pharmacokinetics and safety in congenital thrombotic thrombocytopenic purpura’, Blood, 130: 2055-63.
32. Sedaghat, S., P. S. de Vries, J. Boender, M. A. Sonneveld, E. J. Hoorn, A. Hofman, M. P. do Maat, O. H. Franco, M. A. Ikram, F. W. Leebeek, and A. Dehghan. 2016. ‘von Willebrand Factor, ADAMTS13 Activity, and Decline in Kidney Function: A Population-Based Cohort Study’, Am J Kidney Dis, 68: 726-32.
33. Sonneveld, M. A., M. P. de Maat, M. L. Portegies, M. Kavousi, A. Hofman, P. L. Turecek, H. Rottensteiner, F. Schelflinger, P. J. Koudstaal, M. A. Ikram, and F. W. Leebeek. 2015. ‘Low ADAMTS13 activity is associated with an increased risk of ischemic stroke’, Blood, 126: 2739-46.
34. South, K., M. O. Freitas, and D. A. Lane. 2016. ‘Conformational quiescence of ADAMTS-13 prevents proteolytic promiscuity’, J Thromb Haemost, 14: 2011-22.
35. South et al. 2017. ‘A model for the conformational activation of the structurally quiescent metalloprotease ADAMTS13 by von Willebrand factor’, J Biol Chem, 292: 5760-69.
36. South, K., B. M. Luken, J. T. Crawley, R. Phillips, M. Thomas, R. F. Collins, L. Deforche, K. Vanhoorelbeke, and D. A. Lane. 2014. ‘Conformational activation of ADAMTS13’, Proc Natl Acad Sci USA, 111: 18578-83.
37. South et al. Journal of Thrombosis and Haemostasis, 16: 2289-2299 2018.
38. South K, Freltas M O, Lane D A. A model for the conformational activation of the structurally quiescent metalloprotease ADAMTS13 by von Willebrand factor. J Biol Chem. 2018b; 293(4):1149-50.
39. Stevens, T. R., J. P. James, N. J. Simmonds, D. A. McCarthy, I. F. Laurenson, P. J. Maddison, and D. S. Rampton. 1992. ‘Circulating von Willebrand factor in inflammatory bowel disease’, Gut, 33: 502-6.
40. Stoll, G., and B. Nieswandt. 2019. ‘Thrombo-inflammation in acute ischaemic stroke—implications for treatment’, Nat Rev Neurol, 15: 473-81.
41. Taniguchi, S., T. Hashiguchi, T. Ono, K. Takenouchi, K. Nakayama, T. Kawano, K. Kato, R. Matsushita, M. Nagatomo, S. Nakamura, T. Nakashima, and I. Maruyama. 2010. ‘Association between reduced ADAMTS13 and diabetic nephropathy’, Thromb Res, 125: e310-6.
42. Taylor, A., C. Vendramin, D. Singh, M. M. Brown, and M. Scully. 2020. ‘von Willebrand factor/ADAMTS13 ratio at presentation of acute ischemic brain injury is predictive of outcome’, Blood Adv, 4:398-407.
43. Urisono, Y., A. Sakata, H. Matsui, S. Kasuda, S. Ono, K. Yoshimoto, K. Nishio, M. Sho, M. Akiyama, T. Miyata, K. Okuchi, S. Nishimura, and M. Sugimoto. 2018. ‘Von Willebrand Factor Aggravates Hepatic Ischemia-Reperfusion Injury by Promoting Neutrophil Recruitment in Mice’, Thromb Haemost, 118: 700-08.
44. Wan, H., Y. Wang, J. Ai, S. Brathwalte, H. NI, R. L. Macdonald, E. M. Hol, J. C. M. Meijers, and M. D. I. Vergouwen. 2018. ‘Role of von Willebrand factor and ADAMTS-13 in early brain injury after experimental subarachnoid hemorrhage’, J Thromb Haemost, 16:1413-22.
45. Wang et al., Mol Neurobiol. 2015 December; 52(3): 1572-1579. Published online 2014 Nov. 4. doi: 10.1007/s12035-014-8952-x.
46. Whiteley, W. N., J. Emberson, K. R. Lees, L. Blackwell, G. Albers, E. Bluhmki, T. Brott, G. Cohen, S. Davis, G. Donnan, J. Grotta, G. Howard, M. Kaste, M. Koga, R. von Kummer, M. G. Lansberg, R. I. Lindley, P. Lyden, J. M. Olivot, M. Parsons, D. Toni, K. Toyoda, N. Wahlgren, J. Wardlaw, G. J. Del Zoppo, P. Sandercock, W. Hacke, C. Baigent, and Collaboration Stroke Thrombolysis Trialists. 2016. ‘Risk of intracerebral haemorrhage with alteplase after acute ischaemic stroke: a secondary analysis of an individual patient data meta-analysis’, Lancet Neurol, 15: 925-33.
47. Witsch, T., K. Martinod, N. Sorvillo, I. Portier, S. F. De Meyer, and D. D. Wagner. 2018. ‘Recombinant Human ADAMTS13 Treatment Improves Myocardial Remodeling and Functionality After Pressure Overload Injury in Mice’, J Am Heart Assoc, 7.
48. Wolters, F. J., J. Boender, P. S. de Vries, M. A. Sonneveld, P. J. Koudstaal, M. P. de Maat, O. H. Franco, M. K. Ikram, F. W. Leebeek, and M. A. Ikram. 2018. ‘Von Willebrand factor and ADAMTS13 activity in relation to risk of dementia: a population-based study’, Sci Rep, 8: 5474.
49. Yaghi, S., J. Z. Willey, B. Cucchiara, J. N. Goldstein, N. R. Gonzales, P. Khatri, L. J. Kim, S. A. Mayer, K. N. Sheth, L. H. Schwamm, Council American Heart Association Stroke, Cardiovascular Council on, Nursing Stroke, Cardiology Council on Clinical, Care Council on Quality of, and Research Outcomes. 2017. ‘Treatment and Outcome of Hemorrhagic Transformation After Intravenous Alteplase in Acute Ischemic Stroke: A Scientific Statement for Healthcare Professionals From the American Heart Association/American Stroke Association’, Stroke, 48: e343-e61.
50. Yang, P., Y. Zhang, L. Zhang, Y. Zhang, K. M. Treurniet, W. Chen, Y. Peng, H. Han, J. Wang, S. Wang, C. Yin, S. Liu, P. Wang, Q. Fang, H. Shi, J. Yang, C. Wen, C. Li, C. Jiang, J. Sun, X. Yue, M. Lou, M. Zhang, H. Shu, D. Sun, H. Liang, T. Li, F. Guo, K. Ke, H. Yuan, G. Wang, W. Yang, H. Shi, T. Li, Z. Li, P. Xing, P. Zhang, Y. Zhou, H. Wang, Y. Xu, Q. Huang, T. Wu, R. Zhao, Q. Li, Y. Fang, L. Wang, J. Lu, Y. Li, J. Fu, X. Zhong, Y. Wang, L. Wang, M. Goyal, D. W. J. Dippel, B. Hong, B. Deng, Ybwem Roos, Cblm Majole, J. Liu, and Direct-Mt Investigators. 2020. ‘Endovascular Thrombectomy with or without Intravenous Alteplase in Acute Stroke’, N Engl J Mod, 382: 1981-93.
51. Zhao, B. Q., A. K. Chauhan, M. Canault, I. S. Patten, J. J. Yang, M. Dockal, F. Scheiflinger, and D. D. Wagner. 2009. ‘von Willebrand factor-cleaving protease ADAMTS13 reduces ischemic brain injury in experimental stroke’, Blood, 114: 3329-34.
52. Zhou, S., S. Jiang, J. Guo, N. Xu, Q. Wang, G. Zhang, L. Zhao, Q. Zhou, X. Fu, L. Li, A. Patzak, M. Hultstrom, and E. Y. Lai. 2019. ‘ADAMTS13 protects mice against renal ischemia-reperfusion injury by reducing Inflammation and improving endothelial function’, Am J Physiol Renal Physiol, 316: F134-F45.
53. Zitomersky, N. L., M. Demers, K. Martinod, M. Gallant, S. M. Cifuni, A. Biswas, S. Snapper, and D. D. Wagner. 2017. ‘ADAMTS13 Deficiency Worsens Colitis and Exogenous ADAMTS13 Administration Decreases Colitis Severity in Mice’, TH Open, 1: e11-e23.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. An ADAMTS13 variant having an amino acid sequence comprising one or more amino acid substitutions in the region corresponding to SEQ ID NO:48 relative to the amino acid sequence of wildtype human ADAMTS13.

2. The ADAMTS13 variant according to claim 1, wherein the ADAMTS13 variant comprises substitution at one or more of the following positions relative to the amino acid sequence of wildtype human ADAMTS13: A1144, A1145, A1146, P1147, P1154, P1171, P1173,P1175,P1180, and P1182.

3. The ADAMTS13 variant according to claim 1, wherein the ADAMTS13 variant comprises an amino acid sequence according to SEQ ID NO:50 or 156.

4. The ADAMTS13 variant according to claim 1, wherein the ADAMTS13 variant comprises substitution to valine, isoleucine, or lysine at one or more of the following positions relative to the amino acid sequence of wildtype human ADAMTS13: A1144, A1145, A1146, P1147, P1154, P1171, P1173, P1175, P1180, and P1182.

5. The ADAMTS13 variant according to claim 1, wherein the ADAMTS13 variant comprises:
(i) the amino acid sequence of SEQ ID NO:51, 52, 53, 54, 55, 56, 57, 58, 59, or 60; or
(ii) the amino acid sequence of SEQ ID NO:136, 137, 138, 139, 140, 141, 142, 143, 144, or 145; or
(iii) the amino acid sequence of SEQ ID NO:146, 147, 148, 149, 150, 151, 152, 153, 154, or 155.

6. The ADAMTS13 variant according to claim 1, wherein the ADAMTS13 variant comprises substitution to valine, isoleucine, or lysine at one or both of positions P1180 and/or P1182 relative to the amino acid sequence of wildtype human ADAMTS13.

7. The ADAMTS13 variant according to claim 1, wherein the ADAMTS13 variant comprises the amino acid sequence of SEQ ID NO:59, 60, 144, 145, 154, or 155.

8. The ADAMTS13 variant according to claim 1, wherein the ADAMTS13 variant comprises or consists of: (i) an amino acid sequence having at least 60% sequence identity to the amino acid sequence of SEQ ID NO:61; or (ii) an amino acid sequence having at least 60% sequence identity to the amino acid sequence of SEQ ID NO:62; or (iii) an amino acid sequence having at least 60% sequence identity to the amino acid sequence of SEQ ID NO:63; or (iv) an amino acid sequence having at least 60% sequence identity to the amino acid sequence of SEQ ID NO:64; or (v) an amino acid sequence having at least 60% sequence identity to the amino acid sequence of SEQ ID NO:65; or (vi) an amino acid sequence having at least 60% sequence identity to the amino acid sequence of SEQ ID NO:66; or (vii) an amino acid sequence having at least 60% sequence identity to the amino acid sequence of SEQ ID NO:67; or (viii) an amino acid sequence having at least 60% sequence identity to the amino acid sequence of SEQ ID NO:68; or (ix) an amino acid sequence having at least 60% sequence identity to the amino acid sequence of SEQ ID NO:69; or (x) an amino acid sequence having at least 60% sequence identity to the amino acid sequence of SEQ ID NO:70.

9. The ADAMTS13 variant according to claim 1, wherein the ADAMTS13 variant displays increased proteolytic activity as compared to wildtype human ADAMTS13.

10. A nucleic acid encoding an ADAMTS13 variant according to claim 1.

11. An expression vector, comprising a nucleic acid according to claim 10.

12. A cell comprising an ADAMTS13 variant according to claim 1.

13. A method for producing an ADAMTS13 variant, comprising culturing a cell comprising a nucleic acid according to claim 10 under conditions suitable for expression of an ADAMTS13 variant by the cell.

14. A pharmaceutical composition comprising an ADAMTS13 variant according to claim 1 and a pharmaceutically acceptable carrier, diluent, excipient or adjuvant.

15. (canceled)

16. (canceled)

17. (canceled)

18. A method of treating or preventing a disease or condition characterised by one or more of: an increased level and/or activity of VWF, or of a complex comprising VWF; a reduced level of ADAMTS13; a reduced level of ADAMTS13 proteolytic activity; thrombosis; and inflammation, comprising administering to a subject a therapeutically or prophylactically effective amount of an ADAMTS13 variant according to claim 1.

19. The composition according to claim 18, wherein the disease or condition is selected from: a disease/condition characterised by thrombosis, a disease/condition characterised by inflammation, thrombotic thrombocytopenic purpura (TTP), ischaemic stroke, haemorrhagic stroke, subarachnoid haemorrhage (SAH), intracerebral haemorrhage (ICH), chronic thromboembolic pulmonary hypotension (CTEPH), myocardial infarction (MI), ST-elevation myocardial infarction (STEMI), unstable angina (UA), ischemia, reperfusion, deep venous thrombosis, pulmonary embolism, intravascular coagulation (DIC), hemolytic-uremic syndrome (HUS), cerebral infarction, systemic lupus erythematosus (SLE), disease cause by infection with a SARSr-CoV (e.g. SARS-CoV-2; e.g. COVID-19), acute respiratory distress syndrome (ARDS), pneumonia, kidney damage, nephropathy, microvascular diseases, dementia, Crohn's disease, inflammatory bowel disease, ulcerative colitis, and bacterial diarrhoea.

20. A method of cleaving VWF, comprising contacting VWF or a complex comprising VWF with an ADAMTS13 variant according to claim 1.
